(19) United States
(12) Patent Application Publication
(10) Pub. No.: US 2025/0250302 A1
(43) Pub. Date: Aug. 7, 2025

(54) SYNTHESIS OF SELF-ASSEMBLING ARTIFICIAL PROTEINS UTILIZING A HOST-GUEST SYSTEM AND USES THEREOF

(71) Applicant: Indian Institute of Science Education and Research, Pune (IISER Pune), Pune (IN)

(72) Inventors: Britto S. Sandanaraj, Pune (IN); Ashwini Rajendra Khyade, Pune (IN); Kshitish Chandra Hati, Pune (IN)

(73) Assignee: Indian Institute of Science Education and Research, Pune (IISER Pune), Pune (IN)

(21) Appl. No.: 19/045,344

(22) Filed: Feb. 4, 2025

(30) Foreign Application Priority Data
Feb. 5, 2024 (IN) 202421007772

(51) Int. Cl. C07K 14/00; A61K 47/64; C07K 14/765; C12N 9/76; C12Q 1/37; G01N 33/68

(52) U.S. Cl. CPC C07K 14/001; A61K 47/643; C07K 14/765; C12N 9/6427; C12Q 1/37; G01N 33/6848; C12Y 304/21001; C12Y 304/21062; C12Y 304/21064; G01N 2333/765; G01N 2333/976

(57) ABSTRACT

The invention relates to an efficient method for synthesizing self-assembling artificial proteins (SAPs) utilizing a catalytic host-guest system. The process involves encapsulating hydrophobic probes with cyclodextrin, performing site-specific protein bioconjugation, and employing a second catalysis cycle for enhanced labeling. SAPs produced through this method are versatile and find applications in therapeutic delivery and diagnostics. The process is simplified, scalable, cost-effective, and environmentally sustainable, addressing the challenges of traditional SAP synthesis. By offering improved yield, functionality, and structural integrity, this invention advances the potential of SAPs in medical and industrial fields.

### FIELD OF THE INVENTION

The present invention pertains to the fields of biotechnology, protein engineering, and nanotechnology. Specifically, the present disclosure provides a method for the synthesis of self-assembling artificial proteins (SAPs) utilizing a catalytic host-guest system. The invention finds applications in therapeutic delivery systems, diagnostic tools, and biomolecular engineering.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Synthesis of self-assembling artificial proteins (SAPs) represents a promising avenue for advancements in therapeutic and diagnostic applications. However, existing methodologies are fraught with limitations, including complex procedures, poor scalability, and inefficient purification processes, which hinder their practical utility.

For instance, CN114042170A discloses a carrier protein based on host-guest self-assembly and its application in vaccines. This method employs adamantane-modified carrier proteins in conjunction with beta-cyclodextrin or its derivatives to form host-guest assemblies. While this approach demonstrates improved immunogenicity, it relies on chemical coupling and modification steps that are technically intricate, time-consuming, and dependent on precise control to achieve consistent results. Additionally, the method lacks scalability and robustness for widespread industrial applications.

Similarly, Quan et al. [ACS Nano 2010, 4, 7, 4211-4219] report the design of α-β cyclodextrin dimers connected through “click” chemistry to create noncovalently connected micelles. These micelles are functionalized with RGD peptide ligands for targeted cellular uptake. Despite offering a sophisticated targeting mechanism, this method requires multiple synthetic steps, including ligand protection and deprotection, which complicate the overall process. Furthermore, the thermo-induced release mechanism, while innovative, is not universally applicable and requires stringent control of physiological conditions.

The review by Periasamy R. [J. Carbohyd. Chem., 2021, 40, 135-highlights the state-of-the-art developments in cyclodextrin-based assemblies and their applications. While this review demonstrates the versatility of supramolecular systems in fields such as gene therapy and drug delivery, it also underscores the technical challenges of ensuring precise self-assembly and efficient drug encapsulation. Many of the methods discussed involve significant trade-offs between stability, yield, and scalability.

Additionally, Reddy et al. [ChemBioChem, 2022, 23, e202100607] describe a micelle-assisted protein labeling technology (MAPLabTech) for the synthesis of SAPs. This method employs site-specific labeling of surface-exposed cysteine residues on globular proteins. However, the approach is cumbersome due to the, poor yield, and challenges in purifying the final product. These limitations make the method impractical for large-scale applications.

However, aforementioned methods demonstrate the potential of SAPs but also highlight significant drawbacks such as complexity and scalability where multi-step reactions, chemical modifications, and ligand functionalizations add layers of complexity, making these methods unsuitable for industrial-scale synthesis. Further, need for extensive purification steps leads to significant material loss and increased costs. Also, there is a limited versatility where many approaches are constrained by their reliance on specific reactive intermediates or narrow operational conditions. Nevertheless, use of toxic reagents or the generation of hazardous byproducts raises safety and environmental compliance issues.

Given these challenges, there is a pressing need to develop a simplified, robust, and scalable methodology for synthesizing SAPs. Such a method should not only minimize purification steps but also ensure high yield, structural integrity, and functional consistency of the SAPs. Addressing these limitations would pave the way for broader adoption of SAPs in diverse fields such as drug delivery, vaccine development, and bio-imaging.

### Objective of the Invention

It is an object of the present disclosure to provide a simplified, efficient, and scalable method for synthesizing self-assembling artificial proteins (SAPs).

Another object of the present disclosure is to eliminate cumbersome purification processes typically associated with SAP synthesis.

Yet another object of the present disclosure is to ensure precise site-specific bioconjugation for reliable self-assembly.

Another object of the present disclosure is to enhance the stability, functionality, and scalability of the resulting SAPs.

Yet another object of the present disclosure is to reduce the environmental footprint of the synthesis process by minimizing toxic reagents and waste.

Another object of the present disclosure is to expand the utility of SAPs in therapeutic, diagnostic, and industrial applications.

### SUMMARY OF THE INVENTION

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

An aspect of the present disclosure relates to a method for synthesizing self-assembling artificial proteins (SAPs), the process comprising the steps of:
i. Dissolving the synthetic/chemical probe in an organic solvent to obtain solution A;
ii. Adding 100 mmol γ-cyclodextrin and sodium phosphate buffer (pH 7.4) to solution A.
iii. The above mixture was sonicated for 1-4 hrs at 25-60° C. to obtain solution B;
iv. Protein solution was prepared in sodium phosphate buffer (pH 7.4) in a separate vessel and added to solution B;
v. The above reaction mixture was stirred for 30 mins-48 hours at 20-30° C.; and
vi. Monitoring the reaction mixture using mass spectrometry and obtaining self-assembling artificial proteins (SAPs).

In some embodiments, the sonication is conducted for 2 hours at 40° C.

In some embodiments, the mixture of step (iv) is stirred at a speed of 20 rpm for 16 hours at 25° C.

In some embodiments, the protein is selected from bovine serum albumin (BSA), human serum albumin (HSA), chymotrypsin, subtilisin, and/or proteinase K.

In some embodiments, the cyclodextrin is α, β, or γcyclodextrin.

In some embodiments, the cyclodextrin is γcyclodextrin.

In some embodiments, the synthetic/chemical probe is selected from the following:

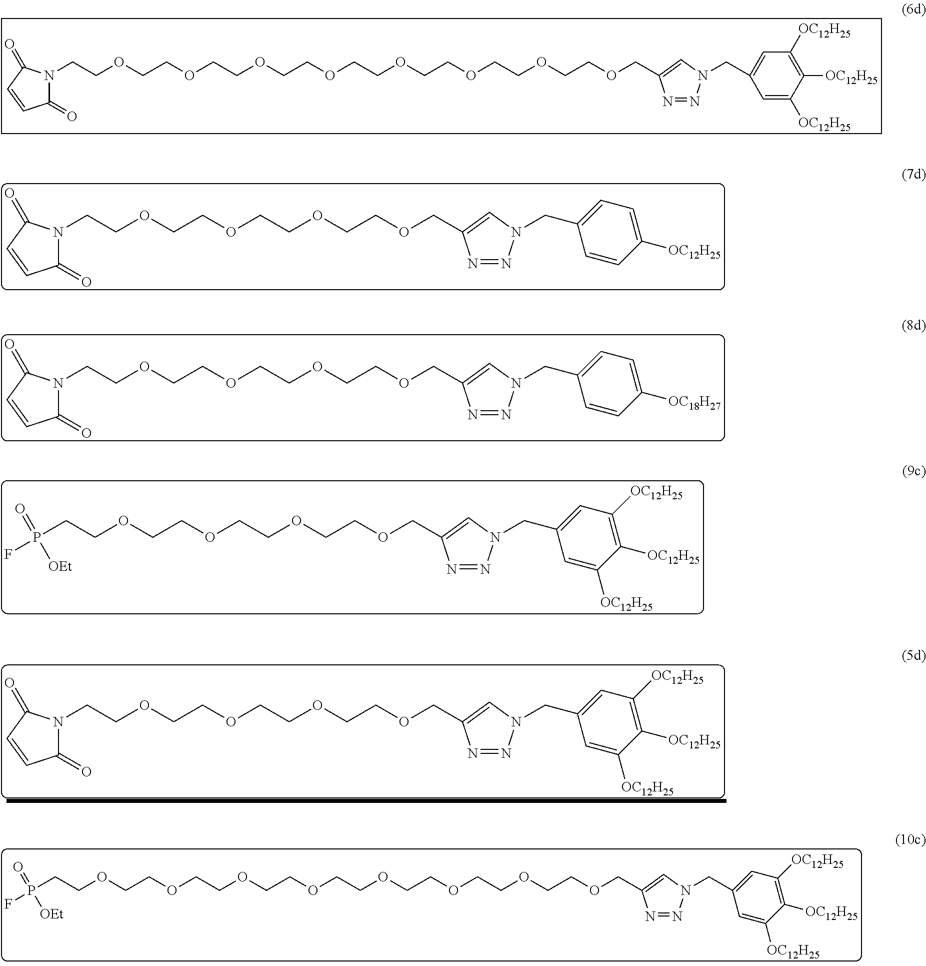

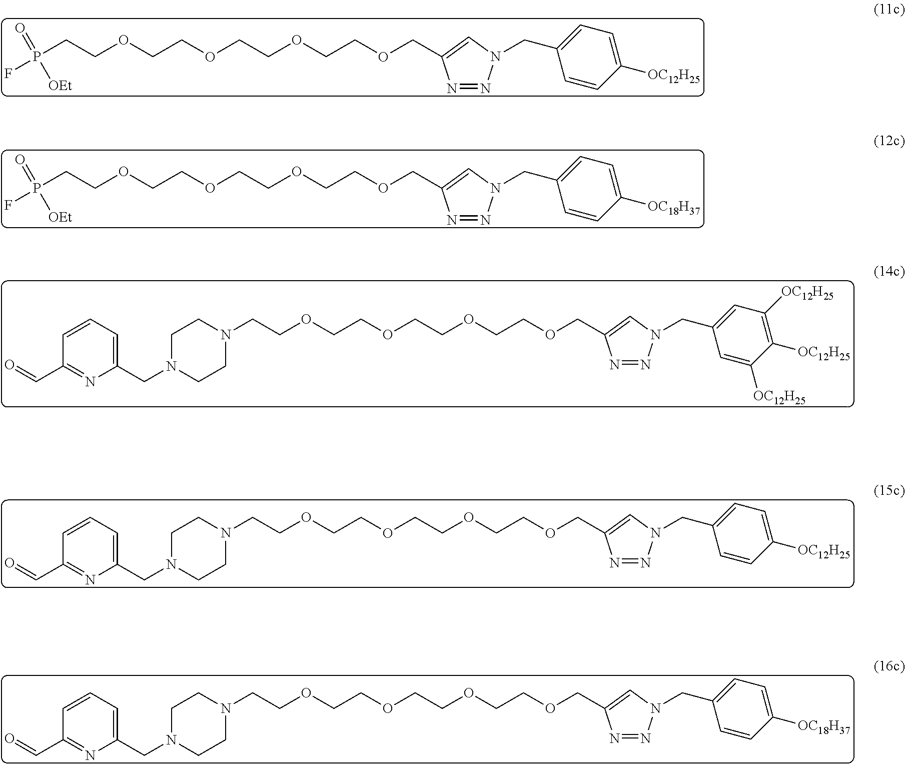

In some embodiments, the SAP is selected from the following:

In some embodiments, the solvent is selected from tetrahydrofuran (THF).

Another aspect of the present disclosure relates to self-assembling artificial protein (SAP) synthesized by the method hereinabove.

Yet another aspect of the present disclosure relates to a composition comprising self-assembling artificial proteins (SAPs) synthesized by the method as claimed hereinabove and a pharmaceutically acceptable carrier.

In some embodiments, the composition is configured for targeted drug delivery or imaging applications.

Various objects, features, aspects and advantages of the inventive subject matter will become more apparent from the following detailed description of preferred embodiments.

### BRIEF DESCRIPTION OF THE DRAWINGS

The accompanying drawing(s) are included to provide a further understanding of the present disclosure and are incorporated in and constitute a part of this specification. The drawings illustrate exemplary embodiments of the present disclosure and, together with the description, serve to explain the principles of the present disclosure. The diagrams are for illustration only, which thus is not a limitation of the present disclosure.

FIG. 1 illustrates schematic representation of Host-Guest Complex Assisted Protein Labelling Technology.

FIG. 2 illustrates inclusion complex formation UV-Vis spectroscopy.

FIG. 3 illustrates MALDI-ToF-MS data of (A) BSA-MI-TEG-C12-3T conjugate (B) HSA-MI-TEG-C12-3T conjugate (C) BSA-MI-OEG-C12-3T conjugate (D) HSA-MI-OEG-C12-3T conjugate.

FIG. 3(E) illustrates structure of conjugates in the spacer variant library.

FIG. 4 illustrates MALDI-ToF-MS data of (A) BSA-MI-TEG-C12-1T conjugate (B) HSA-MI-TEG-C12-1T conjugate (C) BSA-MI-TEG-C18-1T conjugate (D) HSA-MI-TEG-C18-1T conjugate.

FIG. 4(E) illustrates structure of conjugates in the tail variant library

FIG. 5 illustrates MALDI-ToF-MS data of (A) Chy-FP-TEG-C12-3T conjugate (B) Sub-FP-TEG-C12-3T conjugate (C) Pro K-FP-TEG-C12-3T conjugate (D) Chy-FP-OEG-C12-3T conjugate (E) Sub-FP-OEG-C12-3T conjugate (F) Pro K—FP-OEG-C12-3T conjugate.

FIG. 5(G) illustrates structure of conjugates in the spacer variant library.

FIG. 6 illustrates MALDI-ToF-MS data of (A) Chy-FP-TEG-C12-1T conjugate (B) Sub-FP-TEG-C12-1T conjugate (C) Pro-K-FP-TEG-C12-1T conjugate (D) Pro-K-FP-TEG-C18-1T conjugate.

FIG. 6E illustrates structure of conjugates in the tail variant library.

FIG. 7 illustrates MALDI-ToF-MS data of (A) of BSA-NPC-TEG-C12-3T conjugate (B) HSA-NPC-TEG-C12-3T conjugate (C) Chy-NPC-TEG-C12-3T conjugate.

FIG. 7D illustrates structure of conjugates in the protein variant library.

FIG. 8 illustrates MALDI-ToF-MS data of (A) BSA-NPC-TEG-C12-1T conjugate (B) HSA-NPC-TEG-C12-1T conjugate (C) BSA-NPC-TEG-C18-1T conjugate (D) HSA-NPC-TEG-C18-1T conjugate.

FIG. 8E illustrates structure of conjugates in the tail variant library.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, numbers have been used for quantifying weights, percentages, ratios, and so forth, to describe certain embodiments of the invention and are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein that follows, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention.

Groupings of alternative elements or embodiments of the invention disclosed herein are not to be construed as limitations. Each group member can be referred to individually or in any combination with other members of the group or other elements found herein. One or more members of a group can be included in, or deleted from, a group for reasons of convenience and/or patentability. When any such inclusion or deletion occurs, the specification is herein deemed to contain the group as modified.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

Various terms as used herein are shown below. To the extent a term used is not defined below, it should be given the broadest definition persons in the pertinent art have given that term as reflected in printed publications and issued patents at the time of filing.

### Definitions

For the purpose of the present invention, Artificial Proteins (APs) may be defined as an Engineered proteins designed for specific applications through synthetic or semi-synthetic processes.

For the purpose of the present invention, Self-Assembly may be defined as a process where molecules autonomously organize into ordered structures without external guidance.

For the purpose of the present invention, Host-Guest System may be defined as a system in which host molecules, such as cyclodextrins, encapsulate guest molecules to facilitate reactions.

For the purpose of the present invention, Cyclodextrin may be defined as a Cyclic-oligosaccharides that form host-guest complexes with hydrophobic molecules.

For the purpose of the present invention, Hydrophobic Probe may be defined as a molecule that interacts primarily with hydrophobic environments, aiding in protein bioconjugation.

For the purpose of the present invention, Bioconjugation may be defined as a chemical linking of biomolecules, such as proteins or peptides, to other molecules.

For the purpose of the present invention, Protein Engineering may be defined as a design and construction of proteins with novel properties or functions.

For the purpose of the present invention, Phosphate Buffer may be defined as a solution used to maintain a specific pH during biochemical reactions.

For the purpose of the present invention, sonication may be defined as a technique employing ultrasonic waves to facilitate chemical processes.

For the purpose of the present invention, therapeutic applications may be defined as uses in medical treatment, including drug delivery and disease intervention.

For the purpose of the present invention, diagnostic applications may be defined as uses in detecting or monitoring diseases and conditions.

For the purpose of the present invention, micelle formation may be defined as self-assembly of amphiphilic molecules into nanoparticles in aqueous solutions.

For the purpose of the present invention, site-specific labeling may be defined as a process that attaches labels to specific locations within a protein.

For the purpose of the present invention, PEGylation refers to the attachment of polyethylene glycol to molecules to enhance their stability and solubility.

For the purpose of the present invention, immunogenicity may be defined as the ability of a substance to provoke an immune response.

For the purpose of the present invention, redox-sensitive systems refers to the systems responsive to oxidative and reductive environments, often used in drug delivery.

For the purpose of the present invention, host-guest complexation may be defined as an interaction between two molecules, where one serves as the host and the other as the guest.

For the purpose of the present invention, yield may refer to the amount of product obtained from a chemical or biochemical reaction.

The present disclosure relates to self-assembling artificial proteins (SAPs). More particularly, the present disclosure provides a method for the synthesis of self-assembling artificial proteins (SAPs) utilizing a host-guest system. The SAPs of the present disclosure can be used for both therapeutic, diagnostic and preventive (vaccines) applications.

The present disclosure relates to a new method in which a host-guest system is exploited for the synthesis of self-assembling artificial proteins (SAPs). The encapsulation of highly hydrophobic probe molecules by cyclodextrin facilitates site-specific bioconjugation of proteins to make SAPs. Most importantly, this method avoids the use of standard purification methods and hence makes synthesis more attractive.

An aspect of the present disclosure relates to a method for synthesizing self-assembling artificial proteins (SAPs), the process comprising the steps of:
i. Dissolving the synthetic/chemical probe in an organic solvent to obtain solution A;
ii. Adding 100 mmol γ-cyclodextrin and sodium phosphate buffer (pH 7.4) to solution A.
iii. The above mixture was sonicated for 1-4 hrs at 25-60° C. to obtain solution B;
iv. Protein solution was prepared in sodium phosphate buffer (pH 7.4) in a separate vessel and added to solution B;
v. The above reaction mixture was stirred for 30 mins-48 hours at 20-30° C.; and
vi. Monitoring the reaction mixture using mass spectrometry and obtaining self-assembling artificial proteins (SAPs).

In some embodiments, the sonication is conducted at 25-30° C. or 30-35° C. or 35-40° C. or 40-45° C. or 45-50° C. or 50-55° C. or 55-60° C. In some embodiments, the sonication is conducted for 1 hr or 2 hrs or 3 hrs or 4 hrs. In some embodiments, the sonication is conducted for 2 hours at 40° C.

In some embodiments, the mixture of step (iv) is stirred at a speed of 20 rpm for 16 hours at 25° C.

In some embodiments, the mixture of step (iv) is stirred at a speed of 10 rpm, or 12 rpm or 14 rpm or 16 rpm or 18 rpm or 22 rpm, or 24 rpm or 26 rpm or 28 rpm or 30 rpm.

In some embodiments, the protein is selected from bovine serum albumin (BSA), human serum albumin (HSA), chymotrypsin, subtilisin, and/or proteinase K.

In some embodiments, the cyclodextrin is α, β, or γcyclodextrin.

In some embodiments, the cyclodextrin is γcyclodextrin.

In some embodiments, the solvent is a water miscible solvent. In some embodiments, the solvent is selected from DMSO, DMF, acetone, methanol, ethanol, IPA, acetonitrile, 1,4 Dioxane, NMP, tetrahydrofuran (THF) or combinations thereof. In preferred embodiments, the solvent is selected from tetrahydrofuran (THF).

Another aspect of the present disclosure relates to self-assembling artificial protein (SAP) synthesized by the method hereinabove.

Yet another aspect of the present disclosure relates to a composition comprising self-assembling artificial proteins (SAPs) synthesized by the method as claimed hereinabove and a pharmaceutically acceptable carrier.

In some embodiments, the composition is configured for targeted drug delivery or imaging applications.

Various objects, features, aspects and advantages of the inventive subject matter will become more apparent from the following detailed description of preferred embodiments.

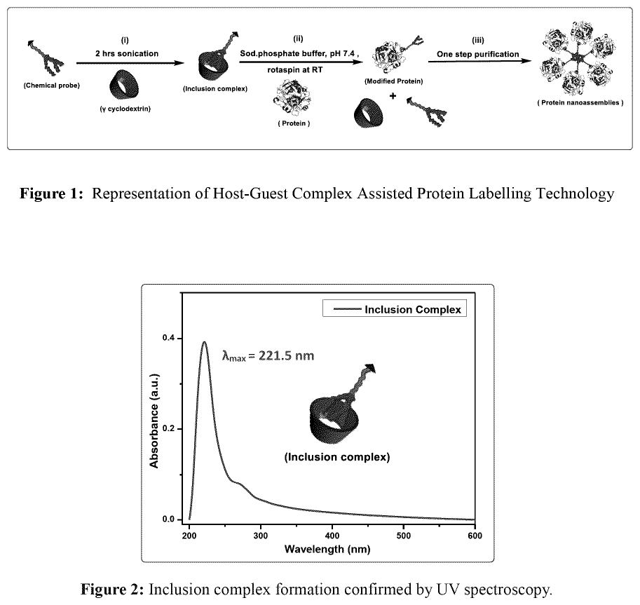

Host-guest complex technology for the synthesis of self-assembling artificial proteins (FIG. 1) was explored by the inventors. In choosing the right host, the inventors focused on the following attributes of the host while exploring host-guest complex technology:
The host should be highly soluble in an aqueous medium;
the host should have a cavity to encapsulate the hydrophobic molecule; and
The host should not non-specifically interact with proteins.

The inventors found cyclodextrin satisfied all the attributes as these compounds were used to solubilize hydrophobic drugs for various applications. Inspired by a study on cyclodextrin's ability to solubilize hydrophobic drugs (not soluble in water), it was hypothesized that it could be used to solubilize the probe of the present invention in an aqueous media.

The inventors then conducted a solubility test of α, β, and γcyclodextrin in water and found that a and γcyclodextrin had better solubility in water compared to β cyclodextrin. The inventors then performed a solubility test of the amphiphilic probe in a and γcyclodextrins and concluded that the probe solubility in γcyclodextrin was better compared to a. This could be because the cavity size of γcyclodextrin is larger than that of a, which helps in solubilizing the hydrophobic tail more effectively. After attempting to solubilize the probe in different concentrations of γ-cyclodextrin, it was found that a 1:2 ratio of the probe to cyclodextrin is optimal for forming inclusion complexes. UV results confirmed the formation of the inclusion complex. (FIG. 2)

The composition comprising pharmaceutically acceptable carriers may be in a form of various oral or parenteral formulations. The composition is formulated with conventional diluents or excipients, including fillers, extenders, binders, wetting agents, disintegrants, and surfactants. Solid formulations for oral administration include tablets, pills, powders, granules, and capsules. These solid formulations are prepared by mixing one or more compounds with at least one of excipients, for example, starch, calcium carbonate, sucrose, lactose, and gelatin. Also, other than simple excipients, lubricants such as magnesium stearate and talc are used as well. Various types of excipients such as wetting agents, sweetening agents, flavors, and preservatives can be included.

### EXAMPLES

The present invention is further explained in the form of following examples. However, it is to be understood that the following examples are merely illustrative and are not to be taken as limitations upon the scope of the invention.

### Procedure for Synthesis of Hydrophilic Spacer

### Synthesis of Tetra Ethylene and Octa Ethylene Glycol Spacer-

In an oven-dried RBF, tetraethylene glycol (TEG) (1.0 eq) was dissolved with stirring in tetrahydrofuran (THF). Sodium hydride (NaH) (1.0 eq) was added to the flask in small portions at 0° C. After 1 hr, propargyl bromide (ProBr) (0.7 eq) was added dropwise, maintaining the reaction at the same temperature. Then, the reaction was stirred for 12 hrs at RT. Upon completion of the reaction, excess NaH was quenched with a dropwise addition of water. The resulting reaction mixture was extracted thrice with dichloromethane (DCM). The combined organic layer was dried over Na₂SO₄ and concentrated under vacuum to get the crude product, which was purified by normal phase chromatography (NPC) using methanol (MeOH)/dichloromethane (DCM) as eluent. In an oven-dried RBF, monopropargylate (1a) (1.0 eq) was dissolved with stirring in THF at 0° C. Aqueous potassium hydroxide (KOH) (1 molar) was added to the flask in small portions immediately. After 30 mins, tosyl chloride (TsCI) (1.0 eq) solution in THF was added dropwise and stirred for 12 hrs. Upon completion, the reaction was quenched with aqueous ammonium chloride and extracted with DCM thrice to get the crude product (1b), which was purified by NPC using MeOH/DCM as an eluent.

In an oven-dried RBF, a mixture of TEG (1.0 eq) and TsO-TEG-Pro (0.5 eq) was taken and dissolved in THF under stirring. The reaction mixture was cooled to 0° C. and NaH (1.0 eq) was added in small portions. The resultant mixture was stirred at RT for 12 hrs. Upon completion of the reaction, excess NaH was quenched by the dropwise addition of water. The resultant mixture was concentrated under reduced pressure. To the obtained residue, water was added and washed with EtOAc thoroughly. Now, the aqueous layer was concentrated under reduced pressure, and to the obtained residue, fresh EtOAc was added and filtered to remove salts. Finally, the collected EtOAc layer was dried over Na₂SO₄ and concentrated under reduced pressure to get product HO-OEG-Pro (1c) along with TEG impurity. This crude product was taken to the next step without purification. Then in an oven-dried RBF, the crude product HO-OEG with TEG impurity (1.0 eq), 4-dimethylamino pyridine (DMAP) (0.5 eq), and TsCl (15 eq) were taken and dissolved in DCM under stirring. The mixture was cooled to 0° C., and triethylamine (Et₃N) (20 eq) was added dropwise. The resultant mixture was then allowed to react for 12 hrs at RT. Upon completion, the reaction was quenched by the dropwise addition of water and extracted thrice with DCM. The combined organic layer was dried over Na₂SO₄ and concentrated under reduced pressure to get a crude product, TsO-OEG-Pro which was purified using NPC to get the pure product (1d). (Scheme 1)

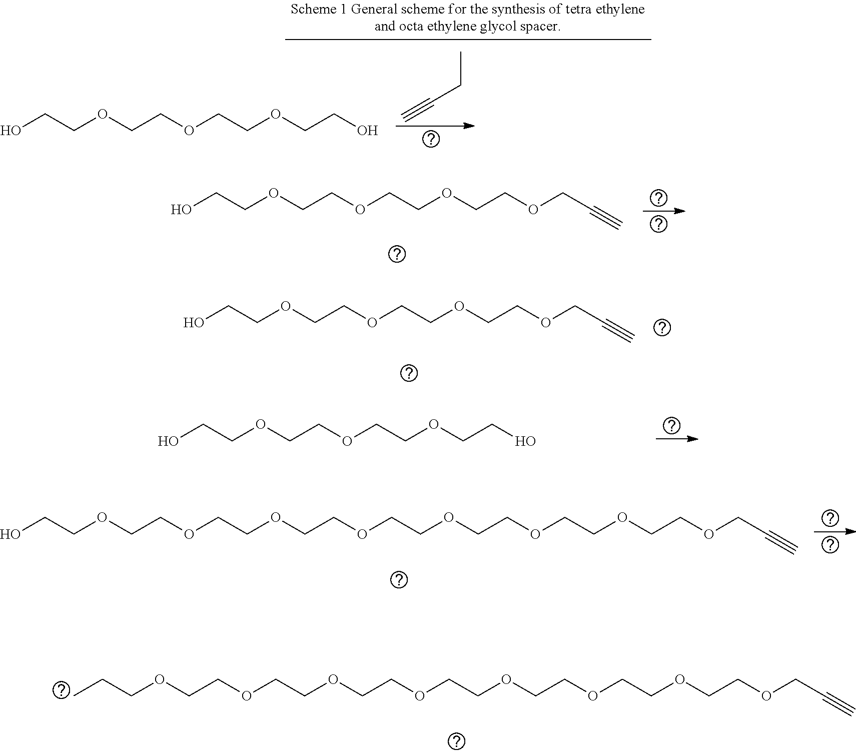

### Procedure for Synthesis of Hydrophobic Tail-

a. Procedure for Synthesis of 3-Tail (3T) Hydrophobic Azide and their Intermediates-

Ethyl 3,4,5-trihydroxybenzoate (1.0 eq), alkyl bromide (RBr) (3.5 eq), K₂CO₃ (3.5 eq), KI (0.05 eq) were taken in an oven-dried RBF. The flask was then purged with nitrogen, and DMF was added under stirring to dissolve the mixture and heated at 70° C. for 12 hrs. Upon completion, the reaction mixture was neutralized with acidic water and extracted thrice with EtOAc. The combined organic layer was dried over Na₂SO₄ and evaporated under vacuum to get the crude product, which was purified using NPC using EtOAc/petroleum ether (PE) to obtain product (2b).

In an oven-dried RBF, the above ester (1.0 eq) was taken and dissolved in THF with stirring at 0° C. Then lithium aluminum hydride (LAH) (3 eq) was added in portions, maintaining the reaction temperature at 0° C. and after 30 mins, allowed to react at RT for 12 hrs. Upon completion, LAH was quenched by the dropwise addition of EtOAc at 0° C. (trans esterification was observed if the temperature was raised). Water was added to the reaction mixture and stirred until the colour changed to white. The resulting solution was filtered using a Buchner funnel, and the residue was washed with EtOAc. The EtOAc layer was separated from water, and the residual water layer was again extracted thrice with EtOAc. The combined organic layers were dried over Na₂SO₄ and concentrated under vacuum to obtain the crude product, which was purified by NPC using EtOAc/PE to obtain product (2c).

In an oven-dried RBF, the above-obtained alcohol (1.0 eq) was dissolved in DCM, and a catalytic amount of dimethylformamide (DMF) was added. Then, thionyl chloride (SOCl₂) (1.6 eq) was added dropwise and allowed to react under stirring for 30 mins at RT. Upon completion of the reaction, DCM and excess SOCl₂ were evaporated under vacuum. The residue was dissolved in diethyl ether (Et₂O) and washed thrice with water. The Et₂O layer was then dried over Na₂SO₄ and concentrated under a vacuum. The resulting crude product (2d) was used for the next step without any purification. In an oven-dried RBF, a mixture of the above obtained chloride (1.0 eq), and sodium azide (NaN₃) (2 eq) was taken, and DMF was added. The resulting mixture was stirred for 12 hrs at RT. Upon completion, water was added to the reaction mixture and extracted thrice with DCM. The combined organic layer was dried over Na₂SO₄ and concentrated under vacuum to get the crude product (2e), which was purified using NPC using EtOAc/PE. (Scheme 2)

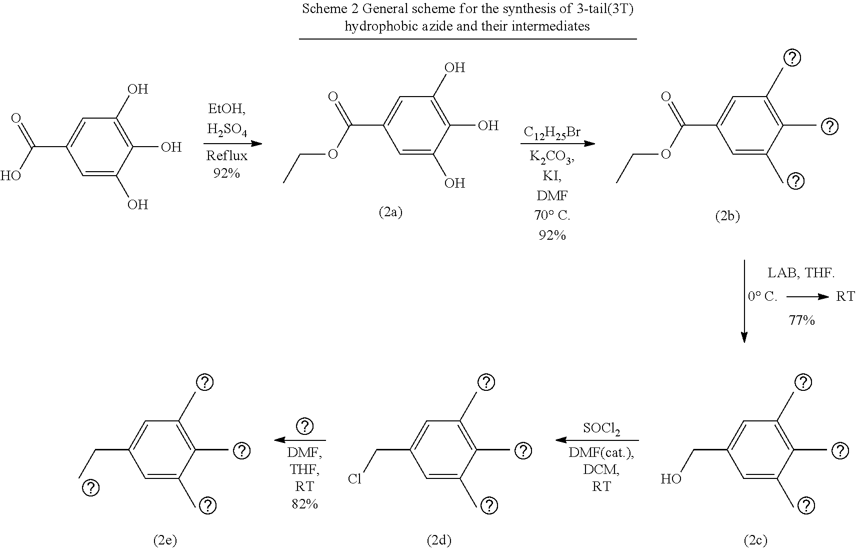

b. Procedure for Synthesis of 1-Tail (1T) Hydrophobic Azide and their Intermediates-

4-hydroxybenzyl alcohol (1.0 eq), RBr (1.1 eq), K₂CO₃ (1.2 eq), and a crown ether (0.1 eq) were combined in an oven-dried round-bottom flask (RBF). The flask was then purged with nitrogen (N₂), and acetone was added under stirring to dissolve the mixture. This mixture was refluxed for 24 hours. Upon completion, the reaction mixture was concentrated and extracted three times with dichloromethane (DCM). The combined organic layer was dried over sodium sulfate (Na₂SO₄) and evaporated under vacuum to yield a crude product, which was purified using normal phase chromatography (NPC) with ethyl acetate (EtOAc) and petroleum ether (PE). Next, in an oven-dried RBF, the obtained alcohol (3a/4a) (1.0 eq) was dissolved in DCM, and a catalytic amount of dimethylformamide (DMF) was added. Sulfuryl chloride (SOCl₂) (1.6 eq) was then added dropwise, and the mixture was stirred for 30 minutes at room temperature (RT). Once the reaction was complete, the DCM and excess SOCl₂ were evaporated under vacuum. The residue was dissolved in diethyl ether (Et₂O) and washed three times with water. The Et₂O layer was dried over Na₂SO₄ and concentrated under a vacuum. The resulting crude product (3b/4b) was used in the next step without further purification. In an oven-dried RBF, the previously obtained chloride (1.0 eq) and sodium azide (NaN₃) (2 eq) were combined, with DMF/TBAB and THE added to the mixture. The resulting mixture was stirred for 12 hours at room temperature. Upon completion, water was added to the reaction mixture, which was then extracted three times with DCM. The combined organic layer was dried over Na₂SO₄ and concentrated under a vacuum to obtain the crude product, which was purified using NPC with EtOAc and PE (3c/4c).

### (Scheme 3)

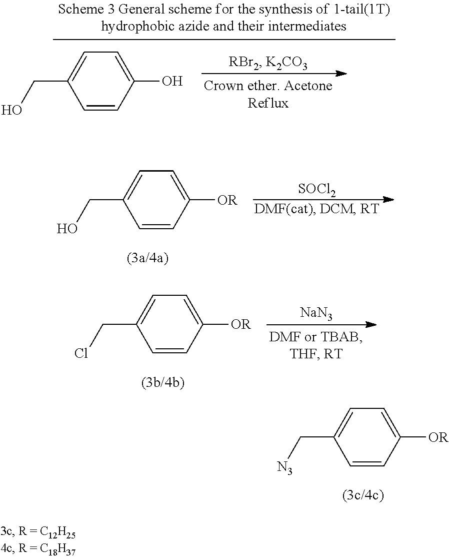

### 3. Procedure for Synthesis of Hydrophobic Chemical Probe-

### A. Thiol-maleimide Chemistry-

Synthesis of chemical probe (5d/6d) using hydrophilic spacer (1b/1d) and hydrophobic three tail (3T) (2e)-

Hydrophilic alkyne (1b/1d) (1 eq) and hydrophobic azide (2e) (4 eq) were dissolved in degassed THF and stirred until a clear solution was obtained, then degassed H₂O was added and stirred vigorously for 10 more mins. Freshly prepared 1M Na ascorbate. (0.05 eq) and 1M CuSO₄ (0.1 eq) was added to the reaction mixture thrice in intervals of 45 mins and allowed to react for 16 hrs at RT. Upon completion, the reaction mixture was extracted with DCM, and the combined organic layer was dried over Na₂SO₄ and concentrated under reduced pressure to get crude product which was purified by NPC using MeOH/DCM solvent system to get clicked product (5a/6a).

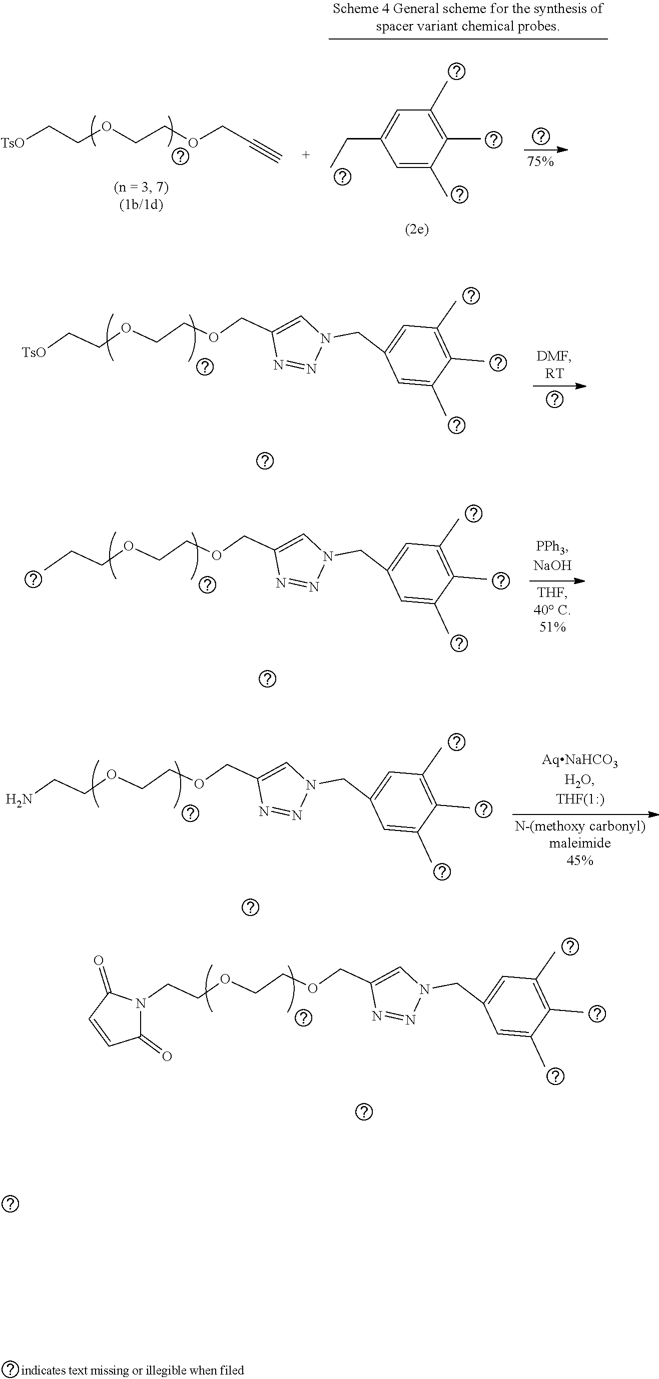

The amphiphilic tosylate (5a/6a) was dissolved in DMF at RT, then NaN₃ (2 eq) was added to the reaction mixture and allowed to react at RT for 12 hrs. After this, the reaction mixture was concentrated and purified by NPC without any workup. Above obtained azide (5b/6b) dissolved in THF then PPh₃ (4 eq) solution in THF was added slowly and stirred at 40° C., after 10 min 1M NaOH was added while maintaining the pH of the solution at 11. The reaction mixture was stirred for 16 hours at 40° C. After completion of the reaction, RM was neutralized with acidic water, followed by DCM extraction, and purified using NPC. Amine (5c/6c) was dissolved in a (1:1) THF: sat. Aq. solution of NaHCO₃ and cooled on an ice bath. Then N-(methoxycarbonyl) maleimide (1.2 eq) was added in portions under stirring. The mixture was stirred for 2 hr at 0° C., followed by 6 hr at 30° C. After extraction with DCM, the organic phase was dried over anhydrous Na₂SO₄, concentrated, and purified by NPC with (MeOH/DCM) yielding the product (5d/6d) (Scheme 4).

Synthesis of Chemical Probe (7d/8d) Using a Hydrophilic Spacer (1b) and Hydrophobic One Tail (1T) (3c/4c)-

Hydrophilic alkyne (1b) (1 eq) and hydrophobic azide (3c/4c) (4 eq) were dissolved in degassed THF and stirred until a clear solution was obtained, then degassed H₂O was added and stirred vigorously for 10 more mins. Freshly prepared 1M Na ascorbate. (0.05 eq) and 1 M CuSO₄ (0.1 eq) was added to the reaction mixture thrice in intervals of 45 mins and allowed to react for 16 hrs at RT. Upon completion, the reaction mixture was extracted with DCM, and the combined organic layer was dried over Na₂SO₄ and concentrated under reduced pressure to get crude product which was purified by NPC using MeOH/DCM solvent system to get clicked product (7a/8a). The amphiphilic tosylate (7a/8a) was dissolved in DMF at RT. Then NaN₃ (2 eq) was added to the reaction mixture and allowed to react at RT for 12 hrs. After this, the reaction mixture was concentrated and purified by NPC without any workup. Above obtained azide (7b/8b) dissolved in THF then PPh₃ (4 eq) solution in THF was added slowly and stirred at 40° C., after 10 min 1M NaOH was added while maintaining the pH of the solution at 11. The reaction mixture was stirred for 16 hours at 40° C. After completion of the reaction, RM was neutralized with acidic water, followed by DCM extraction, and purified using NPC. Amine (7c/8c) was dissolved in a (1:1) THF: sat. Aq. Solution of NaHCO₃ and cooled on an ice bath. Then N-(methoxycarbonyl) maleimide (1.2 eq) was added in portions under stirring. The mixture was stirred for 2 hr at 0° C., followed by 6 hr at 30° C. After extraction with DCM, the organic phase was dried over anhydrous Na₂SO₄, concentrated, and purified by NPC with (MeOH/DCM) yielding the product (7d/8d) (Scheme 5).

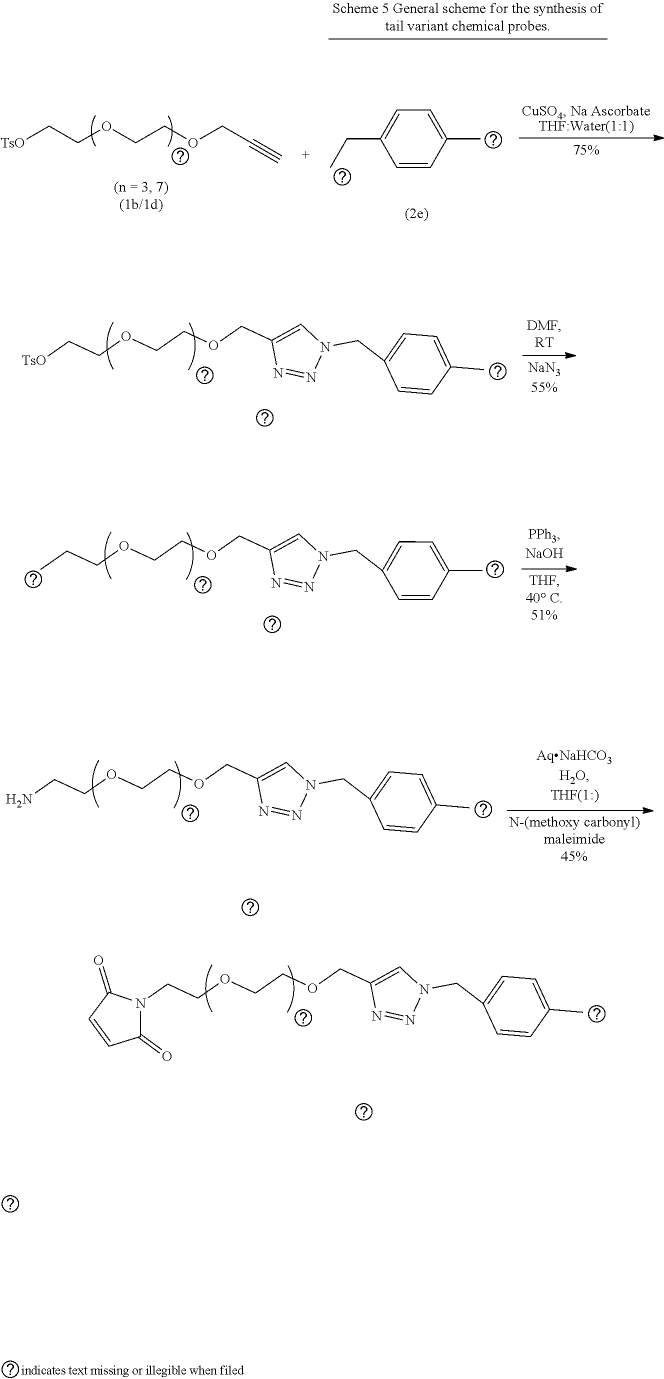

### Host-Guest Complex Assisted Protein Labelling Technology

Bioconjugation of Synthetic Probe (5d/6d) with Proteins (BSA and HSA):

1 mg of the probe was dissolved in 10 μL of tetrahydrofuran (THF). 20 μL of 100 mmol γ-cyclodextrin was added, followed by 470 μL of sodium phosphate buffer (pH 7.4). The reaction mixture was sonicated for 2 hours at 40° C. After sonication, 500 μL of the protein solution, prepared in the same buffer, was added, resulting in a final protein concentration of 50 μM. The reaction mixture was then allowed to rotate at 20 rpm for 16 hours at 25° C. (Scheme 6). After 16 hours, the reaction mixture was analyzed using MALDI-TOF mass spectrometry.

### Monitoring the Bioconjugation Reaction:

15 mg of sinapinic acid was weighed in a microcentrifuge tube. Next, 1.0 ml of matrix solution (a 70:30 mixture of water and acetonitrile with a final concentration of 0.1% TFA) was added and the mixture was vortexed until it was homogeneous. The sample and matrix mixture were then combined at a 1:5 ratio and vortexed for a few seconds. After that, we spotted 1-2 μL of this mixture onto the plate and allowed it to air dry for 15 minutes. Finally, the plate was loaded and analyzed.

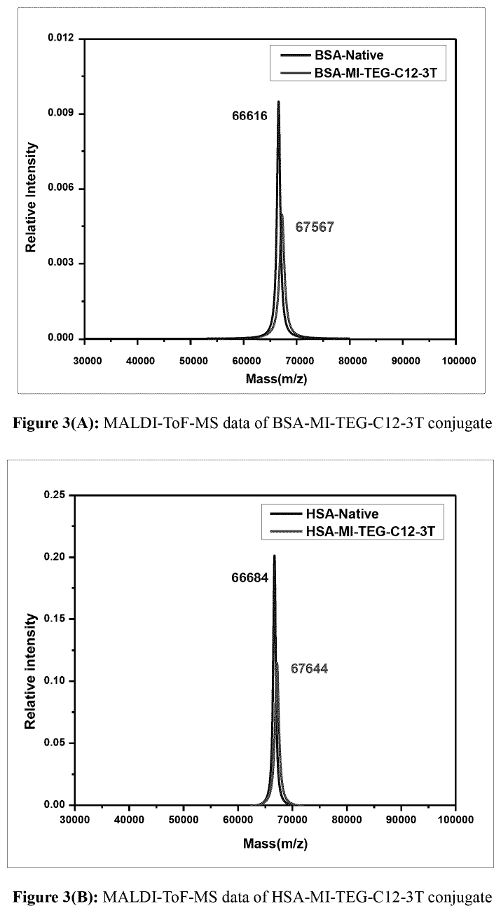

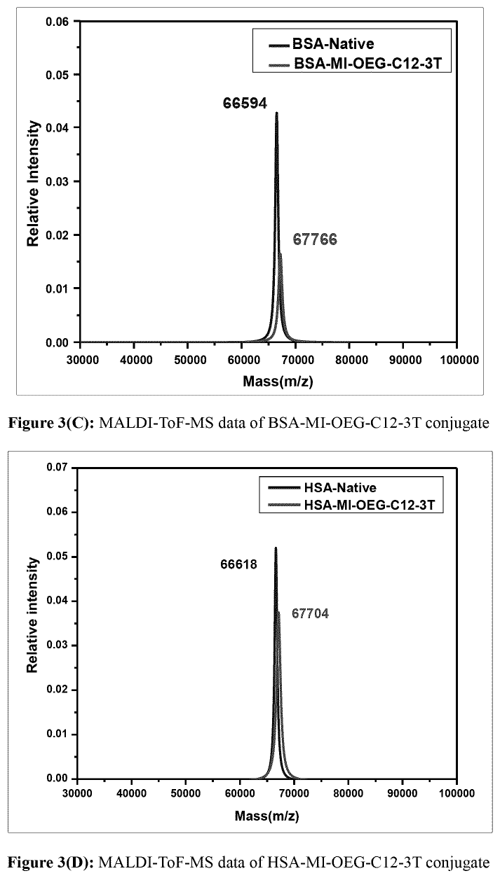

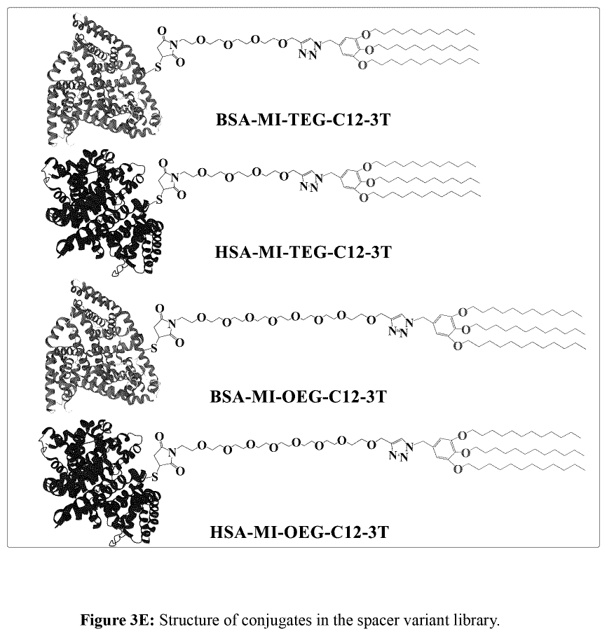

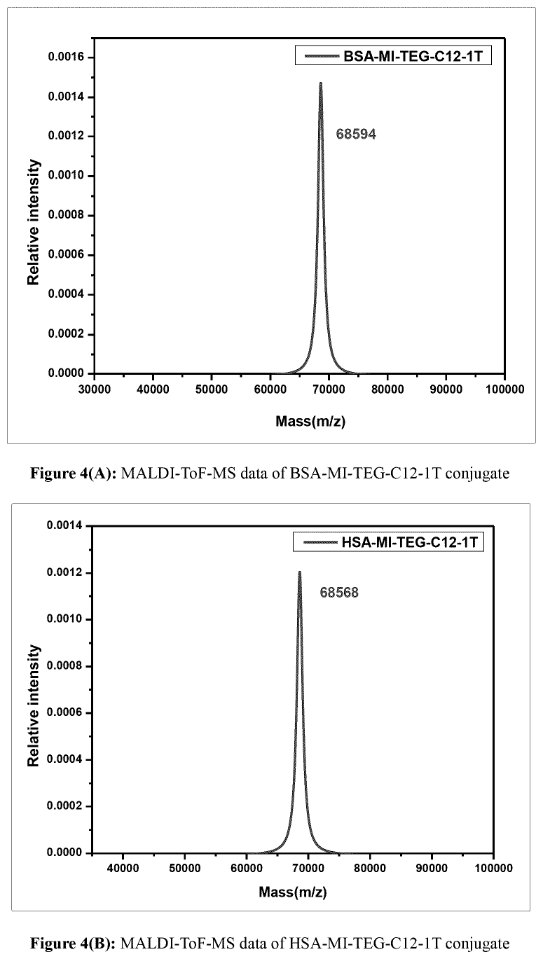

The amphiphilic probe MI-TEG-C12-3T (5d) was allowed to react with BSA and HSA, independently. After the reaction, the reaction mixture was analyzed using MALDI-ToF-MS. A new peak was observed at 67567 Da corresponding to BSA-MI-TEG-C12-3T conjugate (FIG. 3A) and a peak at 67644 Da that corresponds to HSA-MI-TEG-C12-3T conjugate (FIG. 3B). Similarly, for probe MI-OEG-C12-3T (6d), conjugation results for both BSA and HSA were obtained. The new peak for BSA-MI-OEG-C12-3T conjugate (FIG. 3C) appeared at 67766 Da, while the peak for HSA-MI-OEG-C12-3T conjugate (FIG. 3D) was observed at 67704 Da. The structure of protein conjugates is given in (FIG. 3E).

Bioconjugation of Synthetic Probe (7d/8d) with Proteins (BSA and HSA)

5 mg of the probe was dissolved in 10 μL of tetrahydrofuran (THF). Next, 30 μL of 100 mmol γ-cyclodextrin was added, followed by 460 μL of sodium phosphate buffer (pH 7.4). The reaction mixture was sonicated for 2 hours at 40° C. After sonication, 500 μL of the protein solution, prepared in the same buffer, was added, resulting in a final protein concentration of 50 μM. The reaction mixture was then allowed to rotate at 20 rpm for 16 hours at 25° C. (Scheme 7). After 16 hours, the reaction mixture was analyzed using MALDI-TOF mass spectrometry.

### Monitoring the Bioconjugation Reaction

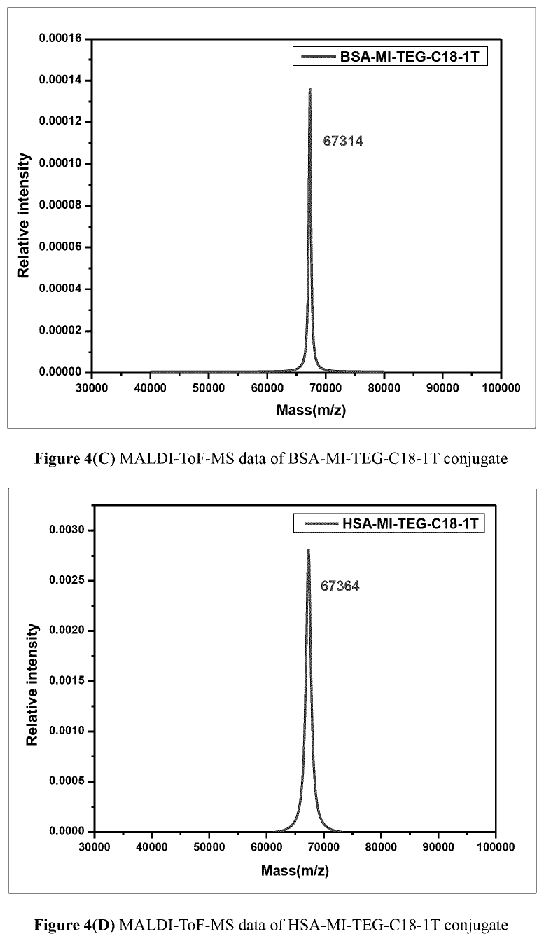

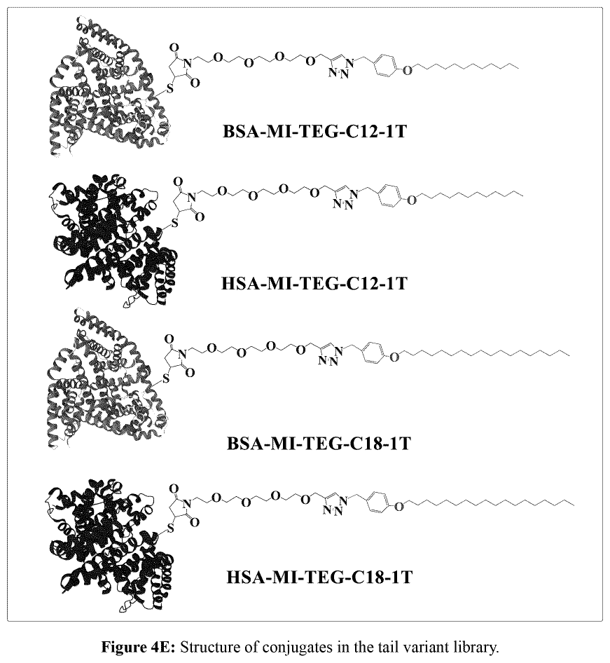

15 mg of sinapinic acid was weighed in a microcentrifuge tube. 1.0 ml of matrix solution (a 70:30 mixture of water and acetonitrile with a final concentration of 0.1% TFA) was added and the mixture was vortexed until it was homogeneous. The sample and matrix mixture were then combined at a 1:5 ratio and vortexed for a few seconds. After that, we spotted 1-2 μL of this mixture onto the plate and allowed it to air dry for 15 minutes. Finally, the plate was loaded and analyzed. The amphiphilic probe MI-TEG-C12-1T (7d) was conjugated with bovine serum albumin (BSA) and human serum albumin (HSA). Surprisingly, a single peak was observed at 68594 Da corresponding to the BSA-MI-TEG-C12-IT conjugate (FIG. 4A) and another peak at 68568 Da corresponding to the HSA-MI-TEG-C12-IT conjugate (FIG. 4B). It is believed that probe (7d) is not very hydrophobic; therefore, the protein conjugates (7e) and (7f) do not self-assemble after conjugation. As a result, the γ-cyclodextrin in the reaction mixture exhibits host-guest interactions with the protein conjugates, which is confirmed by the MALDI-TOF-MS results showing peaks for the protein conjugates plus γ-cyclodextrin. Similarly, for probe MI-TEG-C18-1T (8d), we obtained conjugation results for both BSA and HSA. We observed only one peak for the BSA-MI-TEG-C18-1T conjugate (FIG. 4C), which appeared at 67314 Da, while the peak for the HSA-MI-TEG-C18-1T conjugate (FIG. 4D) was observed at 67364 Da. The MALDI-TOF results show that 100% bioconjugation was obtained. The structure of conjugates is given in (FIG. 4E).

### B. Fluro-Phosphonate Chemistry-

Synthesis of Hydrophilic Spacer (1g/1h)-

A tosylate (1b/1d) (1.0 eq) and potassium iodide (KI) (avoid using unnecessary capital letters) (4.0 eq) were refluxed in acetone for 18
h. Upon completion, excess KI was filtered and washed thrice with acetone. The collected acetone fraction was evaporated under vacuum to get residue, which was then washed with water and extracted with DCM. The combined organic layer was washed with aqueous Na₂CO₃ and then concentrated under vacuum to get the crude product, which was purified by NPC using MeOH/DCM as eluent. In an oven-dried RBF, iodide (1e/1f) (1.0 eq) and triethyl phosphite (P(OEt) 3) (4.0 eq) were taken and refluxed for 1 h. Upon completion of the reaction, the excess P(OEt) 3 was removed under vacuum, and the crude mixture was purified using MeOH/DCM as eluent to obtain (1g/1h). (Scheme 8)

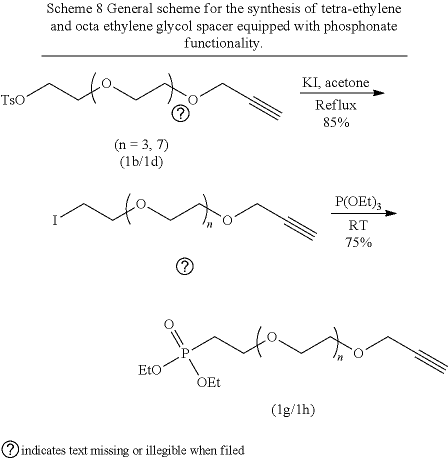

Hydrophobic azide (2e) (4 eq) and hydrophilic alkyne (1g/1h) (1 eq) were dissolved in degassed THF and stirred until a clear solution was obtained, then degassed H₂O was added and stirred vigorously for 10 more mins. Freshly prepared 1M Na ascorbate (0.05 eq) and 1M CuSO₄ (0.1 eq) was added to the reaction mixture thrice in intervals of 45 mins and allowed to react for 16 hrs at RT. Upon completion, the reaction mixture was extracted with DCM, and the combined organic layer was dried over Na₂SO₄ and concentrated under reduced pressure to get crude product which was purified using NPC using the MeOH/DCM solvent system.

Diphosphonate ester (9a/10a) (1 eq) was dissolved in DCM with stirring. Then, Oxalyl chloride (4 eq) was added dropwise at RT and allowed to react for 18 hrs under stirring. Upon completion, excess of Oxalyl chloride and DCM were removed under vacuum. Then, water was added to the residue and stirred for 5 mins. The resulting mixture was extracted thrice with DCM, and the combined organic layer was dried over Na₂SO₄ and concentrated under a vacuum to get the crude product. The crude product was used for the next step without further purification. To the stirring solution of monophosphonate ester (9b/10b) (1 eq) in DCM, DAST (4 eq) was added dropwise at RT and allowed to react for 4 hrs. Excess of DAST and DCM were evaporated using rotary evaporator. To the obtained residue, water was added and stirred for 2 more mins to quench any residual DAST. The reaction mixture was then extracted thrice with DCM. The combined organic layer was dried over Na₂SO₄ and concentrated under a vacuum to get the crude product. The final probe (9c/10c) was used for protein modification without further purification. (Scheme 9)

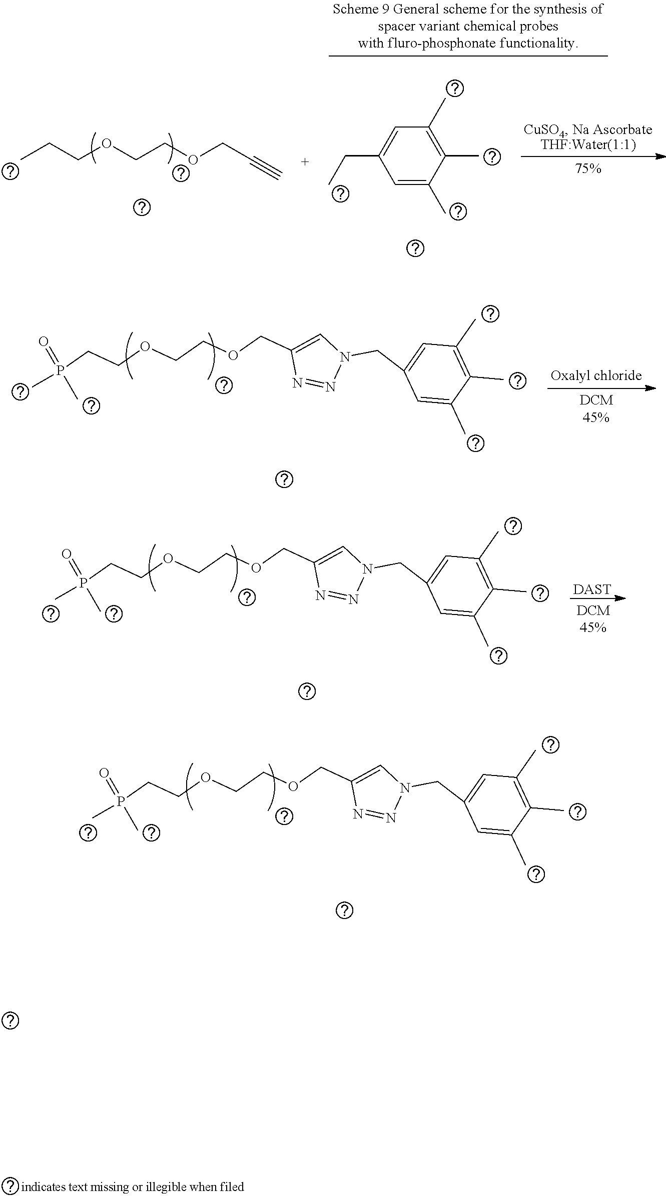

c. Synthesis of Amphiphilic Probe (11c/12c) Using Hydrophilic Spacer (1g) and Hydrophobic Tail (3c/4c)

Hydrophobic azide (3c/4c) (4 eq) and hydrophilic alkyne (1g) (1 eq) were dissolved in degassed THF and stirred until a clear solution was obtained, then degassed H₂O was added and stirred vigorously for 10 more mins. Freshly prepared 1M Na ascorbate (0.05 eq) and 1M CuSO₄ (0.1 eq) were added to the reaction mixture thrice in intervals of 45 mins and allowed to react for 16 hrs at RT. Upon completion, the reaction mixture was extracted with DCM and the combined organic layer was dried over Na₂SO₄ and concentrated under reduced pressure to get crude product which was purified using NPC using MeOH/DCM solvent system.

Diphosphonate ester (11a/12a) (1 eq) was dissolved in DCM with stirring. Then, Oxalyl chloride (4 eq) was added dropwise at RT and allowed to react for 18 hrs under stirring. Upon completion, excess of Oxalyl and DCM were removed under vacuum. Then, water was added to the residue and stirred for 5 mins. The resulting mixture was extracted thrice with DCM, and the combined organic layer was dried over Na₂SO₄ and concentrated under a vacuum to get crude product which was used for the next step without further purification. To the stirring solution of monophosphonate ester (11b/12b) (1 eq) in DCM, DAST (4 eq) was added dropwise at RT and allowed to react for 4 hrs. Excess of DAST and DCM were evaporated under reduced pressure. To the obtained residue, water was added and stirred for 2 more mins to quench any residual DAST. The reaction mixture was then extracted thrice with DCM. The combined organic layer was dried over Na₂SO₄ and concentrated under vacuum to get the crude product. This final probe (11c/12c) was used for protein modification without further purification.

### (Scheme 10)

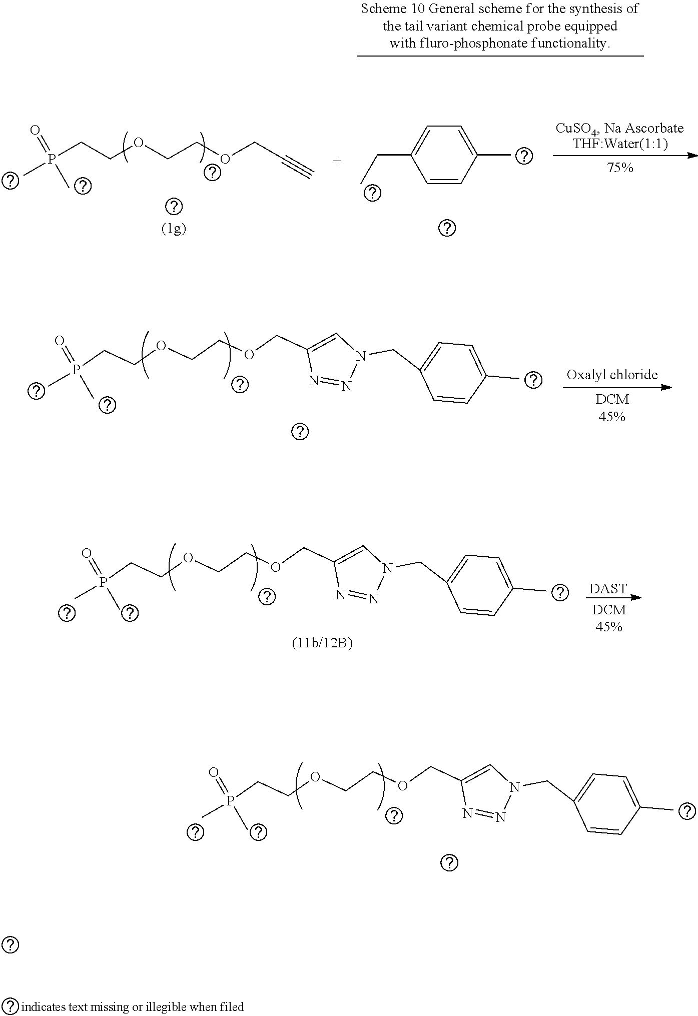

d. Bioconjugation of Synthetic Probe (9c/10c) with Serine Protease

5 mg of the probe was dissolved in 10 μL of tetrahydrofuran (THF). Next, 25 μL of 100 mmol γ-cyclodextrin was added in the probe 9c/10c, followed by 465 μL of sodium phosphate buffer (pH 7.4). The reaction mixture was sonicated for 2 hours at 40° C. After sonication, 500 μL of the protein solution, prepared in the same buffer, was added, resulting in a final protein concentration of 50 μM. The reaction mixture was then allowed to rotate at 20 rpm for 2 hours at 25° C. (Scheme 11). After 2 hours, the reaction mixture was analyzed using MALDI-TOF mass spectrometry.

### Monitoring the Bioconjugation Reaction

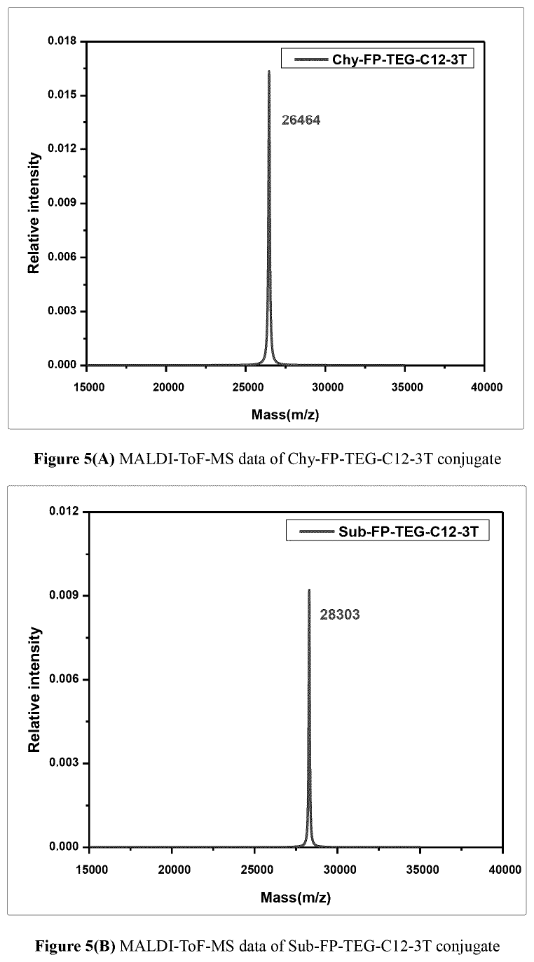

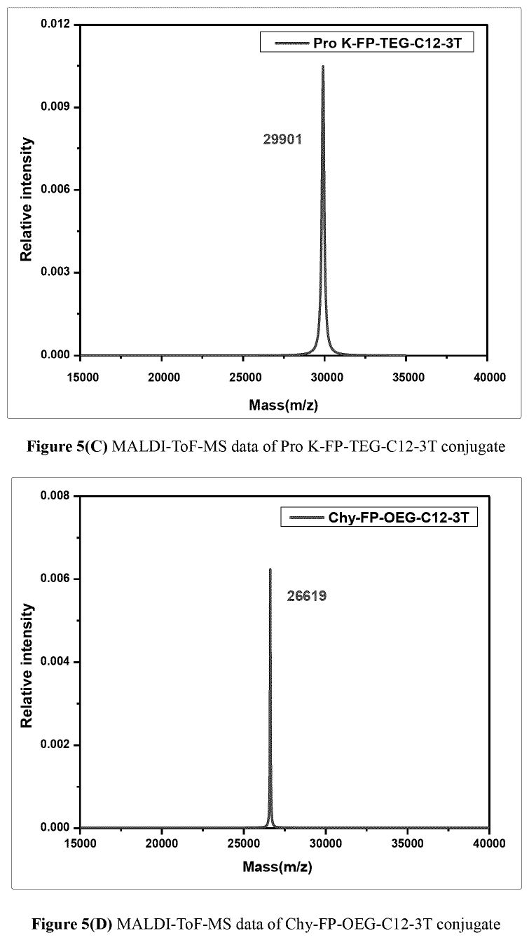

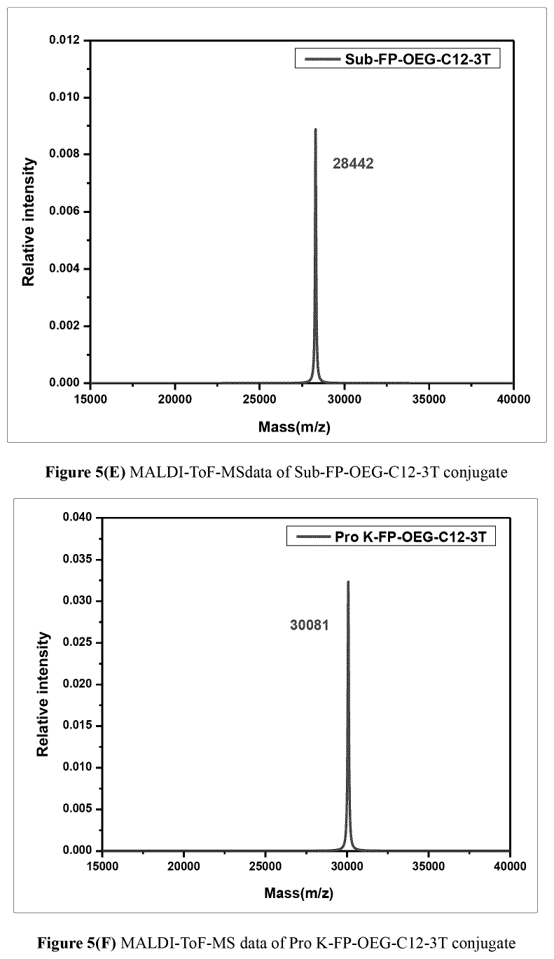

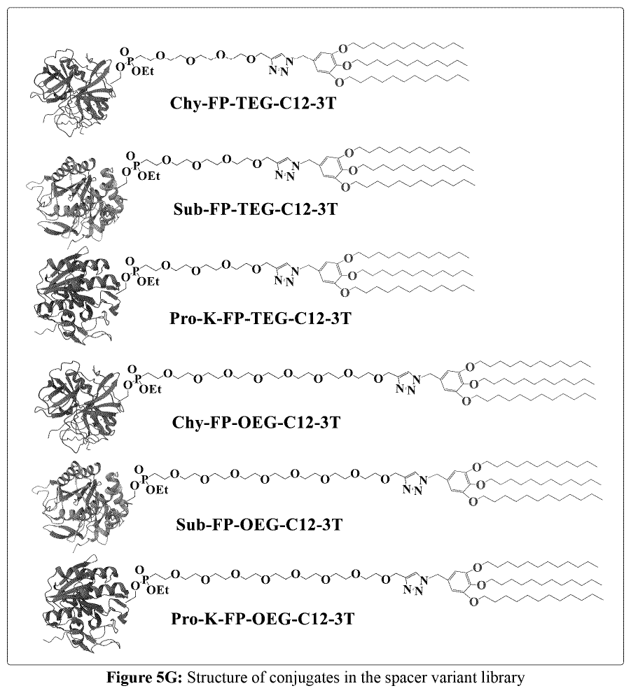

To monitor the extent of protein modification, we directly withdrew samples from the reaction mixture using a pipette for analysis. Specifically, 5 μL of the reaction mixture was combined with 5 μL of 2% TFA and 5 μL of the matrix mixture. The resulting solution was vortexed and then spotted onto a MALDI-TOF MS plate for analysis. Surprisingly, all proteins were successfully conjugated with both probes, as indicated by the absence of peaks for native proteins in the MALDI-TOF data. This confirmed that 100% bioconjugation occurred within 2 hours. For probe FP-TEG-C12-3T (9c), we observed a peak for the chymotrypsin conjugate at 26464 Da, labelled as Chy-FP-TEG-C12-3T (FIG. 5A). We also detected a peak for the subtilisin conjugate at 28303 Da, designated as Sub-FP-TEG-C12-3T (FIG. 5B), and a peak for the proteinase K conjugate at 29901 Da, referred to as Pro K-FP-TEG-C12-3T (FIG. 5C). Similarly, for probe FP-OEG-C12-3T (10c), a peak for the chymotrypsin conjugate was observed at 26619 Da, identified as Chy-FP-OEG-C12-3T (FIG. 5D). Additionally, there was a peak for the subtilisin conjugate at 28442 Da, called Sub-FP-OEG-C12-3T (FIG. 5E) and a peak for the proteinase K conjugate at 30081 Da, named ProK-FP-OEG-C12-3T (FIG. 5F). The structures of the conjugates are illustrated in (FIG. 5G).

d. Bioconjugation of Synthetic Probe (11c/12c) with Serine Protease

5 mg of the probe was dissolved in 10 μL of tetrahydrofuran (THF). Next, 20 μL of 100 mmol γ-cyclodextrin was added, followed by 470 μL of sodium phosphate buffer (pH 7.4). The reaction mixture was sonicated for 2 hours at 40° C. After sonication, 500 μL of the protein solution, prepared in the same buffer, was added, resulting in a final protein concentration of 50 μM. The reaction mixture was then allowed to rotate at 20 rpm for 2 hours at 25° C. (Scheme 12). After 2 hours, the reaction mixture was analyzed using MALDI-TOF mass spectrometry.

### Monitoring the Bioconjugation Reaction

To monitor the extent of protein modification, we directly withdrew samples from the reaction mixture using a pipette for analysis. Specifically, 5 μL of the reaction mixture was combined with 5 μL of 2% TFA and 5 μL of the matrix mixture. The resulting solution was vortexed and then spotted onto a MALDI-TOF MS plate for analysis. To our surprise, all proteins were able to react with probe (11c), as indicated by the absence of peaks for native proteins in the MALDI-TOF data, confirming that 100% bioconjugation occurred within 2 hours.

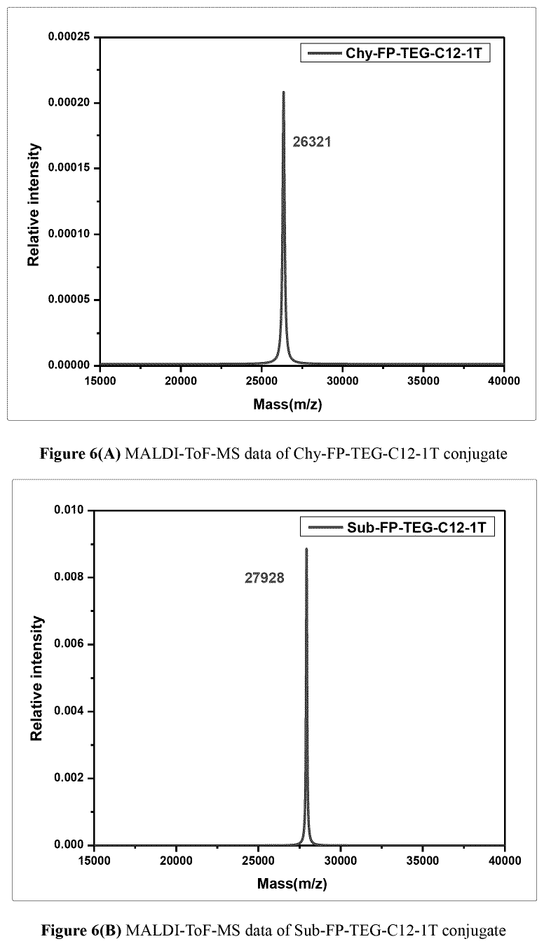

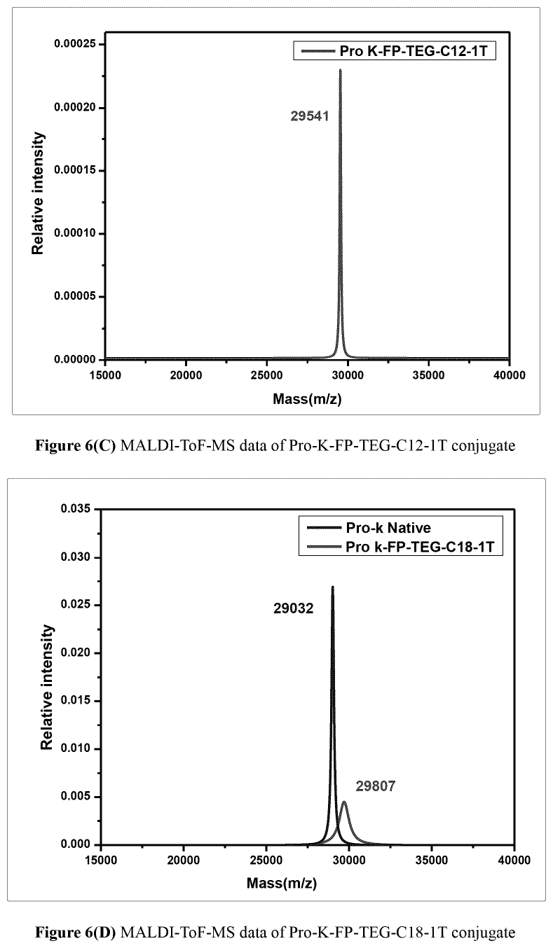

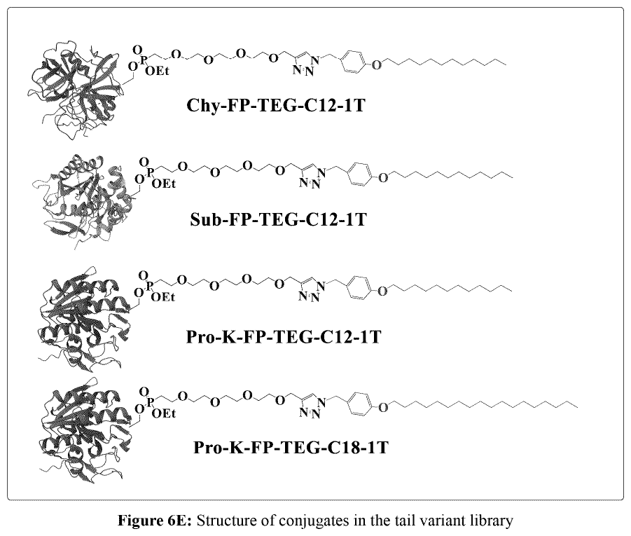

For probe FP-TEG-C12-1T (11c), we observed a peak for the chymotrypsin conjugate at 26321 Da (Chy-FP-TEG-C12-1T) as shown in FIG. 6A. Additionally, there was a peak for the subtilisin conjugate at 27928 Da (Sub-FP-TEG-C12-1T) in FIG. 6B, and a peak for the proteinase K conjugate at 29541 Da (Pro-K-FP-TEG-C12-1T) displayed in FIG. 6C.

For probe FP-TEG-C18-1T (12c), we observed a conjugated peak at 29807 Da (Pro-K-FP-TEG-C18-1T) in FIG. 6D, along with the native proteinase K, indicating successful conjugation. The structure of the conjugates is provided below in FIG. 6E.

### C. N-Terminal Universal Method Synthesis of Hydrophilic Spacer (1i)

Tosylate (1b) (1 eq) and BOC-piperazine (4 eq), K₂CO₃ (2 eq) were dissolved in THF in an RBF. Then the mixture was refluxed at 65° C. for 16 hrs. After completion of the reaction, water was added and extracted with DCM, concentrated, and purified by NPC using MeOH/DCM as a solvent system.

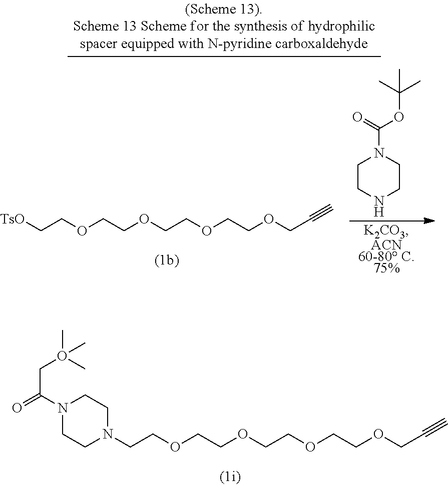

### Synthesis of Pyridinium Intermediate (13b)

To a solution of 2,6-pyridinedimethanol (1 eq) in 1,4-dioxane, selenium dioxide (SeO₂) (0.5 eq) was added. The resulting mixture was sonicated for 5 minutes and then stirred at 80° C. for 24 hrs. Then the reaction was cooled to room temperature and diluted with DCM. The mixture was filtered through celite, and the filtrate was concentrated under reduced pressure. The resulting crude material was purified by NPC using EtOAc/Hexane to get a liquid that became an off-white solid later (13a). After that Mesylchloride (2 eq) was added to the solution of alcohol (13a) (1eq) in DCM at 0° C., Et₃N was added to the above mixture, maintaining the temperature at 0° C., the reaction mixture was stirred for 16 hrs at RT. Upon completion, the reaction mixture was extracted with DCM, and the combined organic layer was dried over Na₂SO₄ and concentrated under reduced pressure to get crude product, which was purified by NPC using EtOAc/Hexane to get mesylate product (13b) (Scheme 14).

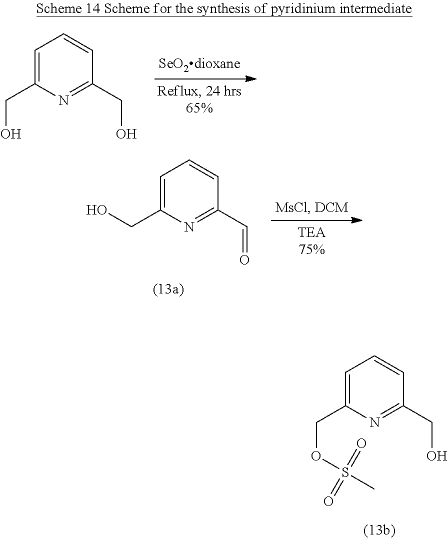

### Synthesis of Amphiphilic Probe (14c) Using Hydrophilic Spacer (1i) and Hydrophobic Tail (2e)

Hydrophilic alkyne (1i) (1 eq) and hydrophobic azide (2e) (4 eq) were dissolved in degassed THF and stirred until a clear solution was obtained, then degassed H₂O was added and stirred vigorously for 10 more mins. Freshly prepared 1M Na ascorbate (0.05 eq) and 1M CuSO₄ (0.1 eq) were added to the reaction mixture thrice in intervals of 45 mins and allowed to react for 16 hrs at RT. Upon completion, the reaction mixture was extracted with DCM, and the combined organic layer was dried over Na₂SO₄ and concentrated under reduced pressure to get the crude product, which was purified by NPC using the MeOH/DCM solvent system to get the clicked product (14a). After that compound (14a) dissolved in DCM and 2 ml, Conc HCl was added to the reaction mixture and stirred for two hrs. After two hrs reaction mixture was quenched by the addition of aq. NaOH solution and extracted by DCM. The organic layer is concentrated and crude used as it is in the next step.

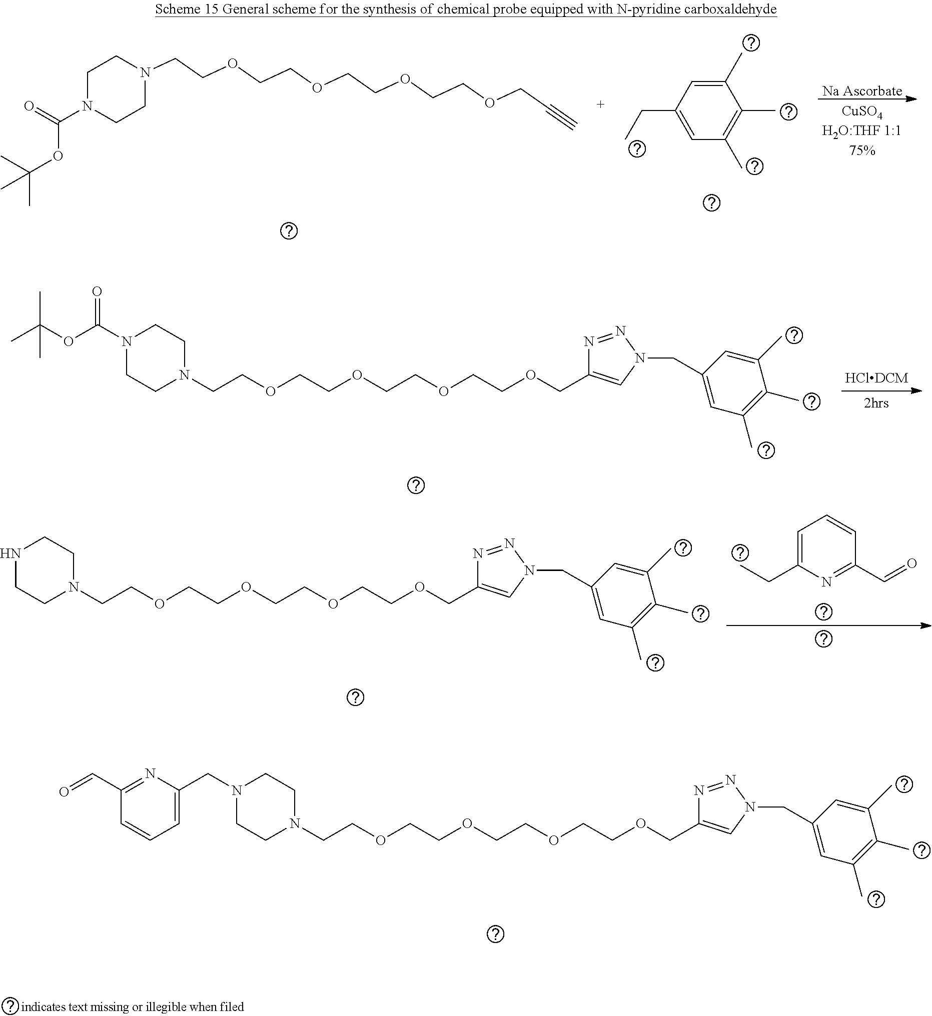

The crude (14b) (1eq), mesylate (13b) (4 eq), and K₂CO₃ (4 eq) were weighed in RBF. Then the mixture was dissolved in ACN and refluxed at 65° C. After 16 hrs, the reaction mixture was concentrated and purified by NPC using MeOH/DCM to get the final compound (14c) (Scheme 15).

Synthesis of Amphiphilic Probe (15c/16c) Using a Hydrophilic Spacer (Li) and a Hydrophobic Tail (3c/4c)

Hydrophilic alkyne (1i) (1 eq) and hydrophobic azide (3c/4c) (4 eq) were dissolved in degassed THF and stirred until a clear solution was obtained, then degassed H₂O was added and stirred vigorously for 10 more mins. Freshly prepared 1M Na ascorbate (0.05 eq) and 1M CuSO₄ (0.1 eq) were added to the reaction mixture thrice in intervals of 45 mins and allowed to react for 16 hrs at RT.

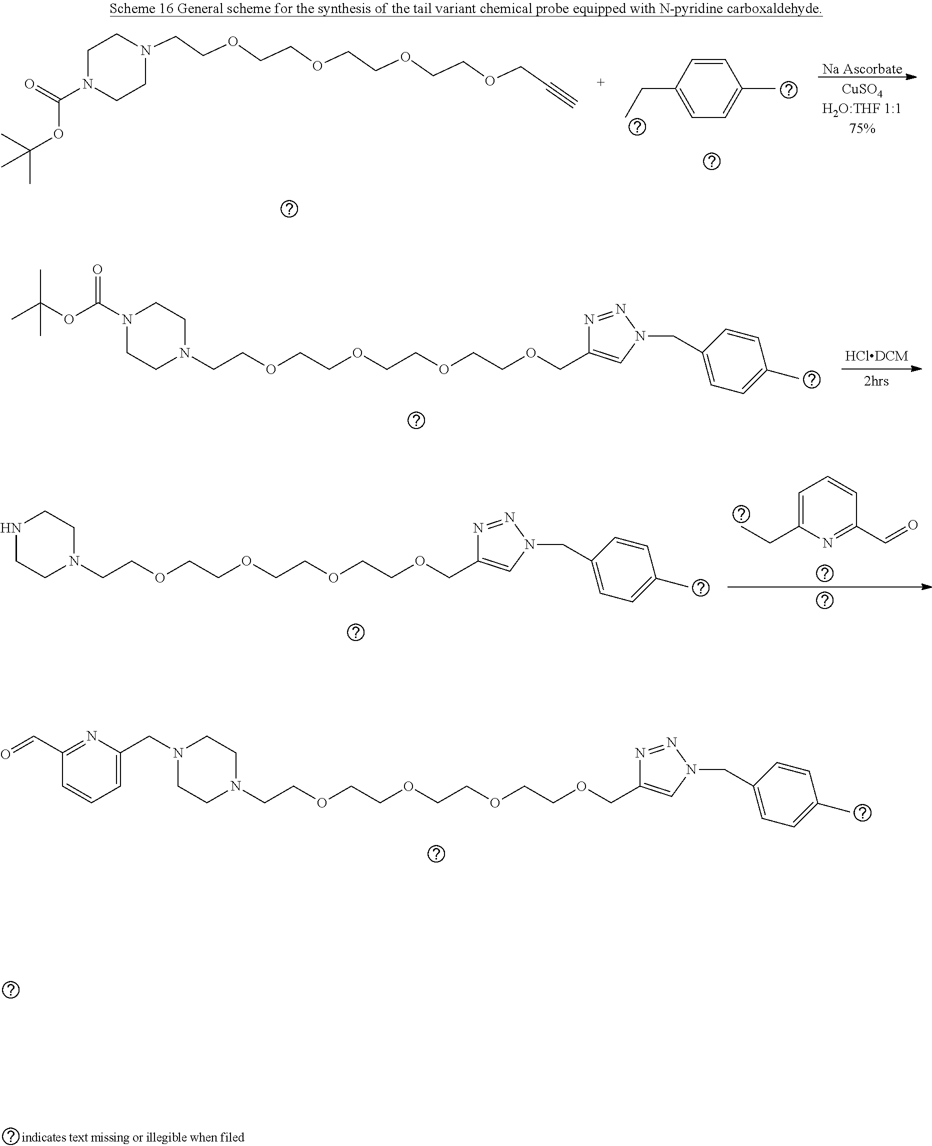

Upon completion, the reaction mixture was extracted with DCM, and the combined organic layer was dried over Na₂SO₄ and concentrated under reduced pressure to get the crude product, which was purified by NPC using MeOH/DCM solvent system to get clicked product (15a/16a). After that compound (15a/16a) dissolved in DCM and 2 ml conc HCl was added to the reaction mixture and stirred for two hrs. After two hrs, reaction mixture was quenched by the addition of aq. NaOH solution and extracted using DCM. The organic layer was concentrated and crude (15b/16b) is used as it is in the next step. The crude (15b/16b) (1eq), mesylate (13b) (4 eq), and K₂CO₃ (4 eq) were weighed in RBF. Then the mixture was dissolved in ACN and refluxed at 65° C. After 16 hrs, the reaction mixture was concentrated and purified by NPC using MeOH/DCM to get the final compound (15c/16c). (Scheme 16)

Bioconjugation of Synthetic Probe (14c) with Proteins (BSA, HSA, and Chy)

5 mg of the probe was dissolved in 10 μL of tetrahydrofuran (THF). Next, 30 μL 5 of 100 mmol γ-cyclodextrin was added, followed by 460 μL of sodium phosphate buffer (pH 7.4). The reaction mixture was sonicated for 2 hours at 40° C. After sonication, 500 μL of the protein solution, prepared in the same buffer, was added, resulting in a final protein concentration of 50 M. The reaction mixture was then allowed to rotate at 20 rpm for 24 hours at 25° C. (Scheme 17). After 24 hours, the reaction mixture was analyzed using MALDI-TOF mass spectrometry.

### Monitoring the Bioconjugation Reaction

For the monitoring the conjugation for BSA and HSA, we weighed 15 mg of sinapinic acid in a microcentrifuge tube. Next, we added 1.0 ml of matrix solution (a 70:30 mixture of water and acetonitrile with a final concentration of 0.1% TFA) and vortexed the mixture until it was homogeneous. The sample and matrix mixture were combined at a 1:5 ratio and vortexed for a few seconds. After that, we spotted 1-2 μL of this mixture onto the plate and allowed it to air dry for 15 minutes. Finally, the plate was loaded and analyzed.

We directly withdrew samples from the reaction mixture using a pipette for analysis to monitor the extent of protein modification for chymotrypsin.

Specifically, 5 μL of the reaction mixture was combined with 5 μL of 2% TFA and 5 μL of the matrix mixture. The resulting solution was vortexed and then spotted onto a MALDI-TOF MS plate for analysis.

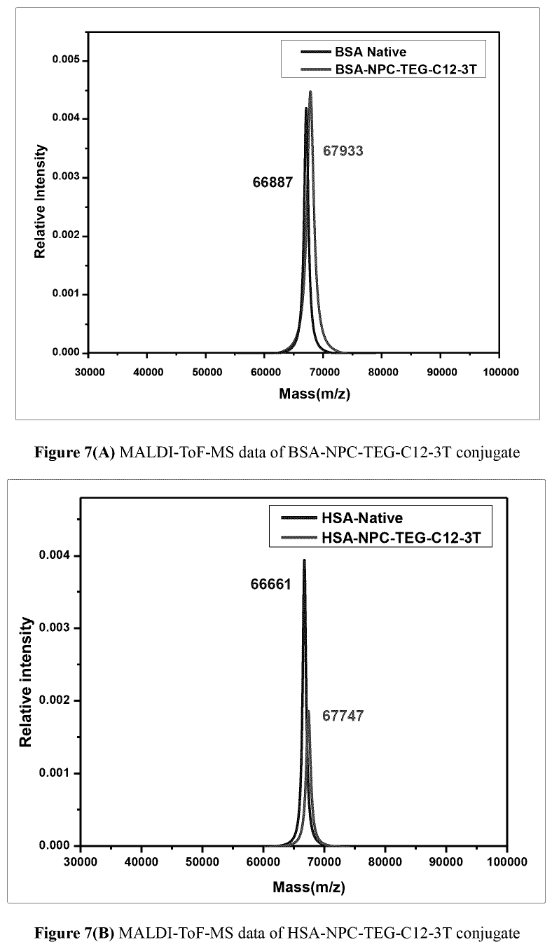

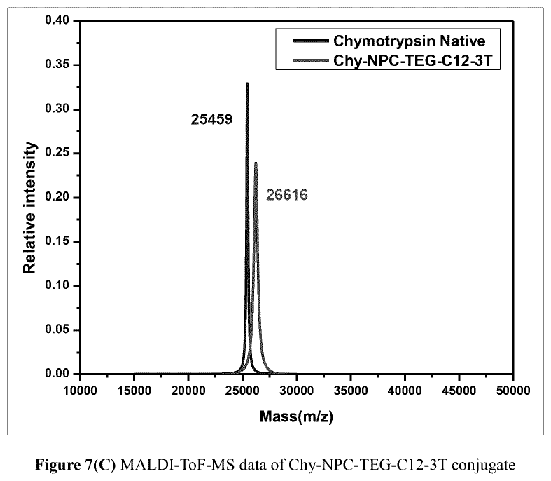

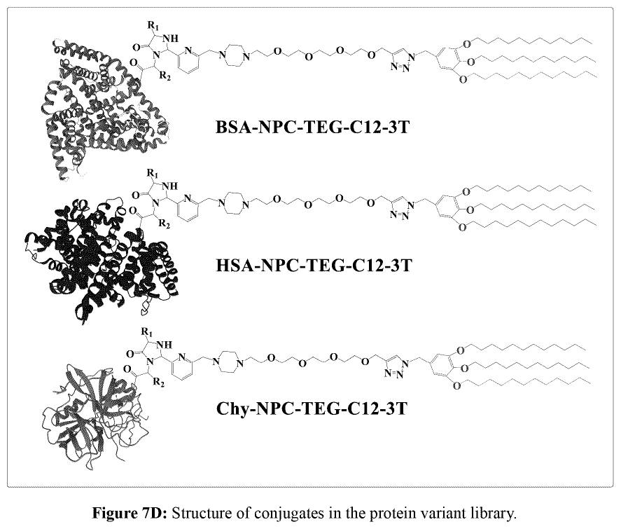

The chemical probe NPC-TEG-C12-3T (14c) was conjugated with BSA, HSA, and chymotrypsin. A new peak was observed at 67933 Da corresponding to BSA-NPC-TEG-C12-3T conjugate (FIG. 7A) and a peak at 67747 Da that corresponds to HSA-NPC-TEG-C12-3T conjugate (FIG. 7B) was also observed. Similarly, a new peak for Chy-NPC-TEG-C12-3T conjugate (FIG. 7C) appeared at 26616 Da. The structure of conjugates is given in (FIG. 7D).

Bioconjugation of Synthetic Probe (15c/16c) with Proteins (BSA and HSA)

5 mg of the probe was dissolved in 10 μL of tetrahydrofuran (THF). Next, 40 μL of 100 mmol γ-cyclodextrin was added, followed by 450 μL of sodium phosphate buffer (pH 7.4). The reaction mixture was sonicated for 2 hours at 40° C. After sonication, 500 μL of the protein solution, prepared in the same buffer, was added, resulting in a final protein concentration of 50 μM. The reaction mixture was then allowed to rotate at 20 rpm for 24 hours at 25° C. (Scheme 18). After 24 hours, the reaction mixture was analyzed using MALDI-TOF mass spectrometry.

### Monitoring the Bioconjugation Reaction

To summarize the procedure, we weighed 15 mg of sinapinic acid in a microcentrifuge tube. Next, we added 1.0 ml of matrix solution (a 70:30 mixture of water and acetonitrile with a final concentration of 0.1% TFA) and vortexed the mixture until it was homogeneous. The sample and matrix mixture were then combined at a 1:5 ratio and vortexed for a few seconds. After that, we spotted 1-2 μL of this mixture onto the plate and allowed it to air dry for 15 minutes. Finally, the plate was loaded and analyzed. Surprisingly, quantitative conjugation was obtained with a probe (15C) for both BSA and HSA protein within 24 hrs.

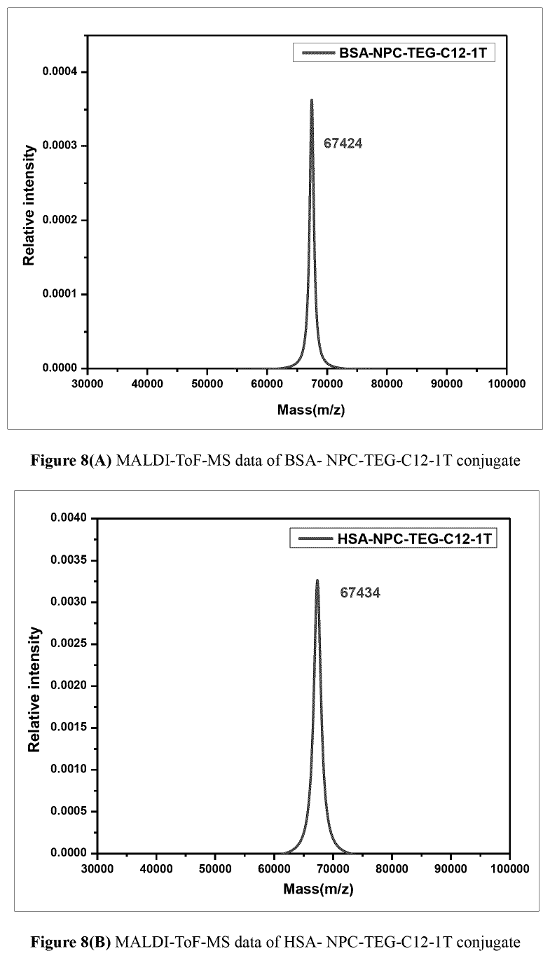

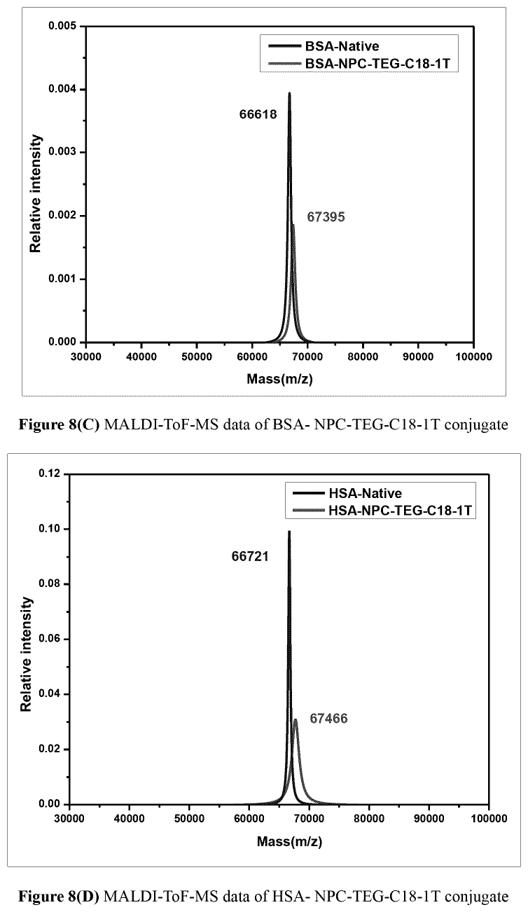

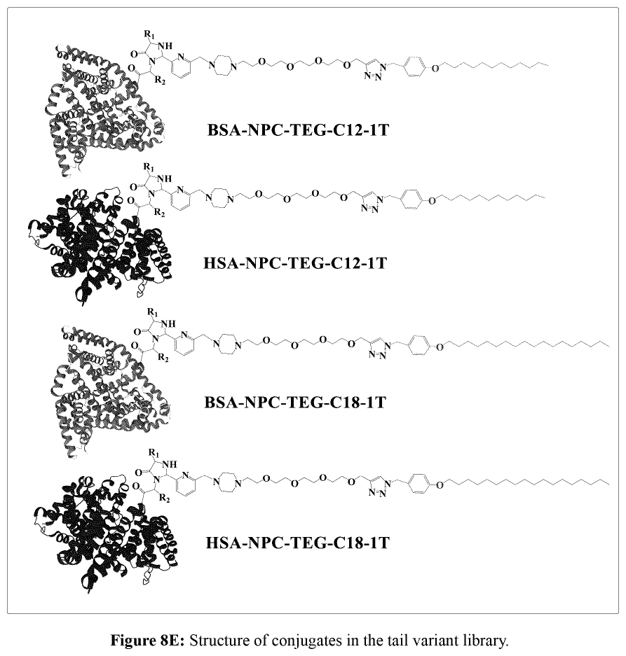

The chemical probe NPC-TEG-C12-1T (15c) was conjugated with BSA and HSA (rewrite this sentence). A single peak was observed at 67424 Da corresponding to BSA-NPC-TEG-C12-1T conjugate (FIG. 8A) and a peak at 67434 Da corresponding to HSA-NPC-TEG-C12-1T conjugate (FIG. 8B). For probe NPC-TEG-C18-1T (16c), conjugation results for both Bovine Serum Albumin (BSA) and Human Serum Albumin (HSA) were obtained. The new peak for the BSA-NPC-TEG-C18-1T conjugate (FIG. 8C) appeared at 67395 m/z, while the peak for the HSA-NPC-TEG-C18-1T conjugate (FIG. 8D) was observed at 67466 Da, alongside the native peak. The structures of the protein conjugates are shown in (FIG. 8E).

### Synthesis of Individual Compounds

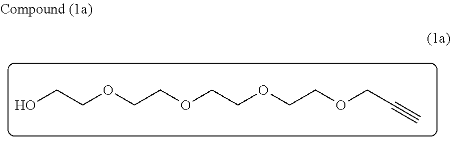 [SMILES: C#CCOCCOCCOCCOCCO]

Mol. formula: C₁₁H₂₀O₅
Mol. weight: 232.13 g/mol
Physical appearance: pale yellow liquid
Yield: 79%

The compound (1a) was synthesized using TEG (7.5 g, 38 mmol), NaH (0.61 g, 26 mmol), and ProBr (3.1 g, 20.6 mmol) in THF. The final product was obtained as a pale-yellow liquid weighing 4.8 g (20.6 mmol, 79% yield). Purification was achieved through NPC with a MeOH/DCM eluent, R_{f} value=0.42 in a 5% MeOH/DCM mixture.

¹H NMR (400 MHZ, CDCl₃) δ : 4.04-4.02 (m, 2H), 3.53-3.48 (m, 14H), 3.42 (ddt, J=5.5,
4.0, 1.6 Hz, 2H), 3.14 (s, 1H), 2.37 (s, 1H).

¹³C NMR (100 MHZ, CDCl₃) δ : 86.35, 84.54, 84.41, 84.22, 83.90, 81.58, 79.28, 77.21, 77.16,
77.12, 76.97, 76.93, 75.69, 68.13, 64.97, 36.27.

HRMS (M+Na): 255.12 g/mol.

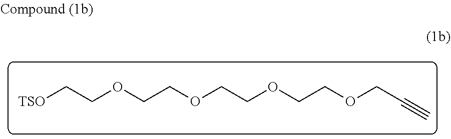

Mol. formula: C₁₈H₂₆O₇S
Mol. weight: 386.45 g/mol
Physical appearance: pale yellow liquid
Yield: 72.4%

The compound (1b) was prepared from compound (1a) (3.5 g, 15 mmol), TsCl (2.1 g, 15 mmol), and KOH (1M) (5 ml) in THF. The product was obtained as a pale-yellow liquid (4.2 g, 10.8 mmol, 72.4%) after purification by NPC using MeOH/DCM as eluent, R_{f} value=0.79 in 5% MeOH/DCM.

¹H NMR (400 MHz, CDCl₃) δ : 7.81-7.77 (m, 2H), 7.35-7.31 (m, 2H), 4.19 (d, J=2.3 Hz,
2H), 4.17-4.14 (m, 2H), 3.70-3.66 (m, 6H), 3.62 (q, J=1.5 Hz, 4H), 3.58 (d, J=1.4 Hz,
4H), 2.44 (s, 3H), 2.42 (t, J=2.4 Hz, 1H).

¹³C NMR (400 MHZ, CDCl₃) δ : 144.89, 133.12, 129.92, 128.07, 79.75, 74.63, 70.83, 70.68,
70.62, 70.48, 69.35, 69.20, 68.77, 58.48, 29.77, 21.73.

HRMS (M+Na): 409.13 g/mol.

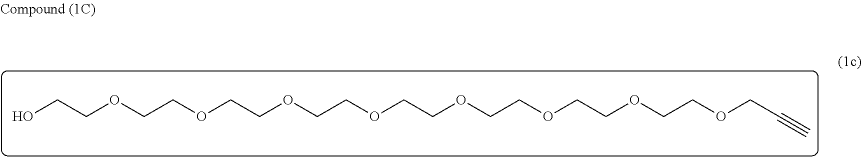 [SMILES: C#CCOCCOCCOCCOCCOCCOCCOCCOCCO]

Mol. formula: C₁₉H₃₆O₉
Mol. weight: 408.48 g/mol
Physical appearance: Pale yellow liquid
Yield: not determined
   The compound (1c) was prepared from compound (1b) (7.3 g, 18.8 mmol), TEG (9.0 g, 46.3 mmol), and NaH (0.9 g, 37.5 mmol) in THF. The product obtained was carried to the next step without purification, R_{f} value=0.32 in 5% MeOH/DCM.

MALDI-TOF MS (M+H): 409.24 g/mol

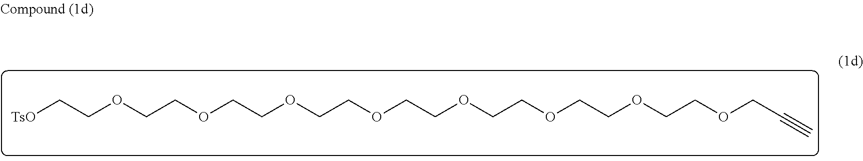 [SMILES: C#CCOCCOCCOCCOCCOCCOCCOCCOCCOS(=O)(=O)c1ccc(C)cc1]

Mol. formula: C₂₆H₄₂O₁₁S
Mol. weight: 562.67 g/mol
Physical appearance: pale yellow liquid
Yield: not determined
   The compound (1d) was prepared from compound (1c) (23 g), TsCl (109.22 g, 573.08 mmol), Et₃N (77.25 g, 764.11 mmol) and DMAP (2.3 g, 19.10 mmol) in DCM. The product was
obtained as pale-yellow liquid (12 g, 21.3 mmol) after purification by NPC using MeOH/DCM as eluent, R_{f} value=0.44 in 5% MeOH/DCM.

¹H NMR (400 MHZ, CDCl₃) δ : 7.78 (d, J=8.0 Hz, 2H), 7.33 (d, J=8.0 Hz, 2H), 4.20-4.17
(m, 2H), 4.16-4.13 (m, 2H), 3.70-3.65 (m, 6H), 3.64-3.62 (m, 16H), 3.60 (s, 4H), 3.57 (s, 4H), 2.43 (s, 4H).

¹³C NMR (100 MHZ, CDCl₃) δ : 144.91, 133.10, 129.94, 128.09, 74.67, 70.83, 70.69, 70.67,
70.65, 70.59, 70.49, 69.35, 69.21, 68.78, 21.75.

MALDI-TOF MS (M+K): 601.11 g/mol.

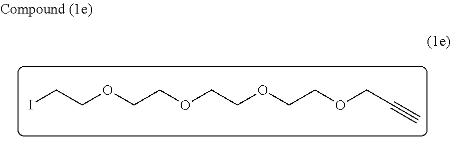 [SMILES: C#CCOCCOCCOCCOCCI]

Mol. formula: C11H19I04
Mol. weight: 342.17 g/mol
Physical appearance: pale yellow liquid
Yield: 85%

The compound (l) was synthesized from compound (1b) (8.06 g, 20.85 mmol), KI (13.90 g, 83.43 mmol) reflexed in acetone. The product was obtained as a pale-yellow liquid (6.02 g, 17.60 mmol, 85%) after purification by NPC using MeOH/DCM as eluent, R_{f} value=0.75 in 5% MeOH/DCM.

¹H NMR (400 MHZ, CDCl₃) δ : 4.20 (d, J=2.4 Hz, 2H), 3.75 (dd, J=7.3, 6.5 Hz, 2H), 3.72-3.62 (m, 14H), 3.26 (dd, J=7.4, 6.5 Hz, 2H), 1.68 (s, 1H).

HRMS (M+Na): 365.02 g/mol.

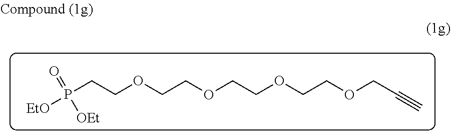 [SMILES: C#CCOCCOCCOCCOCCP(=O)(OCC)OCC]

Mol. formula: C₁₅H₂₉O₇P
Mol. weight: 352.36 g/mol
Physical appearance: pale yellow liquid
Yield: 75%
   The compound (1g) was prepared from (1e) (0.840 g, 2.46 mmol) and P(OEt) 3 (1.63 g, 9.84 mmol). The product was obtained as a pale-yellow liquid (0.649 g, 1.84 mmol, 75%) after purification by NPC using MeOH/DCM as eluent, R_{f} value=0.40 in 5% MeOH/DCM.

¹H NMR (400 MHZ, CDCl₃) δ : 4.17 (d, J=2.3 Hz, 2H), 4.06 (ddq, J=14.5, 7.2, 4.1, 3.5 Hz,
4H), 3.72-3.59 (m, 12H), 2.41 (t, J=2.4 Hz, 1H), 2.10 (dt, J=18.7, 7.5 Hz, 2H), 1.28 (d, J=
7.0 Hz, 4H).

¹³C NMR (100 MHz, CDCl₃) δ : 79.70, 77.36, 74.64, 70.66, 70.65, 70.53, 70.47, 70.25, 69.16,
65.18, 64.64, 64.57, 61.76, 61.70, 61.53, 58.46, 31.98, 29.75, 29.42, 27.72, 26.33, 22.75, 19.51,
18.09, 16.52, 16.46, 14.18, 6.61, 6.54.

HRMS (M+Na): 375.15 g/mol.

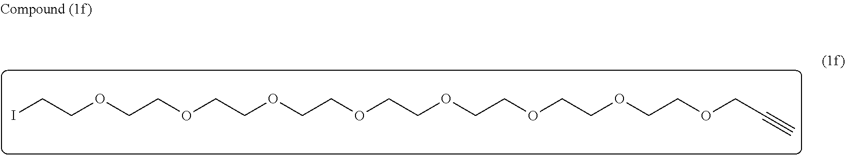 [SMILES: C#CCOCCOCCOCCOCCOCCOCCOCCOCCI]

Mol. formula: C₁₉H₃₅IO₈
Mol. weight: 518.39 g/mol
Physical appearance: pale yellow liquid
Yield: 72%
   The compound (1f) was made from compound (1d) (2 g, 3.55 mmol), KI (2.3 g, 14.21 mmol) reflexed in acetone. The product was obtained as a pale-yellow liquid (1.32 g, 2.55 mmol, 72%)
after purification by NPC using MeOH/DCM as eluent, R_{f} value=0.69 in 5% MeOH/DCM.

¹H NMR (400 MHZ, CDCl₃) δ : 4.20 (d, J=2.4 Hz, 2H), 3.75 (t, J=6.9 Hz, 2H), 3.71-3.63 (m, 28H), 3.29-3.23 (m, 2H), 2.43 (t, J=2.4 Hz, 1H).

¹³C NMR (100 MHZ, CDCl₃) δ : 79.81, 74.68, 72.13, 70.81, 70.73, 70.55, 70.37,
69.26, 58.55, 3.06.

MALDI-TOF MS (M+K): 557.03 g/mol.

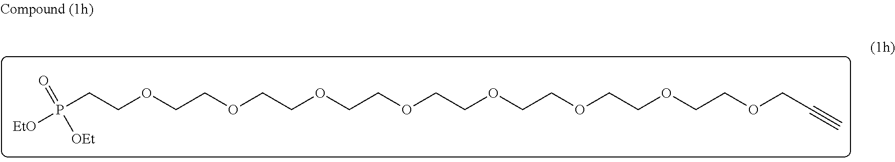 [SMILES: C#CCOCCOCCOCCOCCOCCOCCOCCOCCP(=O)(OCC)OCC]

Mol. formula: C23H45011P
Mol. weight: 528.58 g/mol
Physical appearance: pale yellow liquid
Yield: 75%
   The compound (1h) was prepared from (1f) (0.600 g, 1.1 mmol) and P(OEt) 3 (0.730 g, 4.4 mmol). The product was obtained as a pale-yellow liquid (0.436 g, 0.825 mmol, 75%) after purification by NPC using MeOH/DCM as eluent, R_{f} value=0.40 in 5% MeOH/DCM.

¹H NMR (400 MHZ, CDCl₃) δ : 4.21-4.11 (m, 2H), 4.07 (ddd, J=11.3, 7.2, 3.6 Hz, 4H), 3.78-3.52 (m, 30H), 2.11 (dt, J=18.7, 7.5 Hz, 2H), 1.31 (t, J=7.1 Hz, 7H).

¹³C NMR (100 MHZ, CDCl₃) δ : 74.69, 74.66, 70.69, 70.66, 70.55, 70.50, 70.28, 69.21, 65.23, 61.78, 61.72, 58.51, 29.79, 27.76, 26.38, 16.57, 16.51.

MALDI-TOF MS (M+K): 567.15 g/mol.

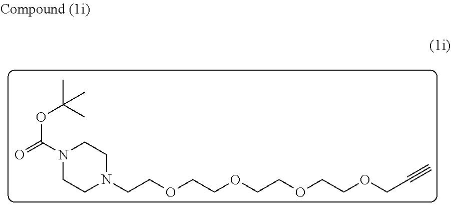 [SMILES: C#CCOCCOCCOCCOCCN1CCN(C(=O)OC(C)(C)C)CC1]

Mol. formula: C₂₀H₃₆N₂O₆
Mol. Weight: 400.52 g/mol
Physical appearance: brownish liquid
Yield: 60%
   Tosylate (1b) (5.26 g, 13.61 mmol), BOC-piperazine (3.8 g, 20.41 mmol), and K₂CO₃ (3.7 g, 27.22 mmol) were dissolved in THF in an RBF. Then, the mixture was refluxed at 65° C. to purify crude to get the pure product (4.90 g, 12.24 mmol, 60%) using NPC and MeOH/DCM as eluent. R_{f} Value=0.45 in 5% MeOH/DCM

¹H NMR (400 MHZ, CDCl₃) δ : 4.14 (d, J=2.4 Hz, 2H), 3.67-3.52 (m, 15H), 3.42-3.33 (m, 4H), 2.61 (s, 1H), 2.53 (d, J=11.4 Hz, 2H), 2.39 (dt, J=6.2, 3.4 Hz, 5H), 1.39 (s, 9H).

¹³C NMR (100 MHZ, CDCl₃): 79.63, 79.56, 74.62, 70.57, 70.54, 70.38, 70.32, 69.25, 69.07, 68.65, 58.37, 57.83, 53.32, 28.41.

MALDI-TOF MS (M+Na): 423.56 g/mol.

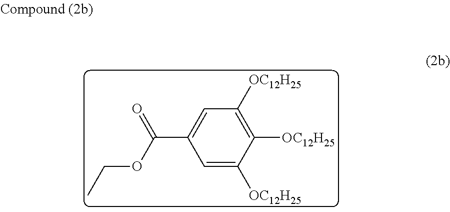

Mol. formula: C₄₅H₈₂H₅
Mol. weight: 702.61 g/mol
Physical appearance: White solid
Yield: 92%
   The compound (2b) was prepared from ethyl 3,4,5-trihydroxy ester (1.0 g, 5.08 mmol), 1-dodecyl bromide (5.93 g, 17.78 mmol), K₂CO₃ (2.45 g, 17.78 mmol), KI (0.042 g, 0.254 mmol) in DMF. The product was obtained as a white solid (3.2 g, 4.6 mmol, 92%) after purification by NPC using EtOAc/PE as eluent, R_{f} value=0.56 in 5% EtOAc/PE.

¹H NMR (400 MHZ, CDCl₃) δ : 7.25 (s, 2H), 4.35 (q, J=7.1 Hz, 2H), 4.01 (t, J=6.5 Hz, 6H), 1.85-1.71 (m, 6H), 1.46 (q, J=7.9 Hz, 6H), 1.38 (t, J=7.1 Hz, 6H), 1.34-1.21 (m, 48H), 0.91-0.84 (m, 9H).

¹³C NMR (100 MHZ, CDCl₃) δ : 166.64, 152.94, 142.45, 125.19, 108.13, 73.64, 69.33, 61.11, 32.09, 32.08, 30.47, 29.90, 29.88, 29.85, 29.81, 29.79, 29.72, 29.55, 29.52, 29.47, 26.24, 26.21,22.84, 14.55, 14.26.

MALDI-TOF MS (M+Na): 725.66 g/mol.

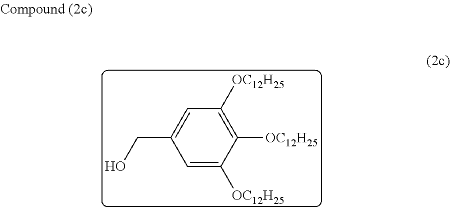

Mol. formula: C₄₃H₈₀O₄
Mol. weight: 660.60 g/mol
Physical appearance: Whitesolid
Yield: 77%
   The compound (2c) was prepared from compound (2b) (0.190 g, 0.27 mmol), and LAH (0.031 g, 0.81 mmol) in THF. The product was obtained as a white solid (0.137 g, 0.21 mmol, 77%) after purification by NPC using EtOAc/PE as eluent, R_{f} value=0.30 in 10% EtOAc/PE.

¹H NMR (400 MHZ, CDCl₃) δ : 6.56 (s, 2H), 4.59 (d, J=4.2 Hz, 2H), 3.95 (dt, J=14.8, 6.6 Hz, 6H), 1.82-1.71 (m, 6H), 1.46 (dd, J=10.3, 5.0 Hz, 6H), 1.34-1.25 (m, 49H), 0.90-0.86 (m, 9H).

¹³C NMR (100 MHz, CDCl₃) δ : 153.08, 137.13, 136.48, 105.07, 73.45, 68.99, 65.20, 32.01, 32.00, 30.37, 29.84, 29.82, 29.80, 29.78, 29.75, 29.70, 29.54, 29.51, 29.47, 29.45, 26.21, 22.75, 14.12.

MALDI-TOF MS (M+Na): 683.45 g/mol.

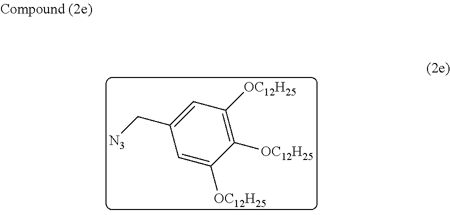

Mol. formula: C₄₃H₇₉O₃
Mol. weight: 685.61 g/mol
Physical appearance: White solid
Yield: 82%
   The compound (2d), a chloride was achieved using compound (2c) (0.250 g, 0.378 mmol), SOCl₂ (0.0714 g, 0.605 mmol), and a catalytic amount of DMF in DCM. Subsequently, azide, compound (2e) was synthesized using NaN₃ (0.049 g, 0.765 mmol) in DMF from compound (2c) without purification. The product was obtained as a white solid (0.212 g, 0.309 mmol, 82%) after purification by NPC using EtOAc/PE as eluent, R_{f} value=0.58 in 5% EtOAc/PE.

¹H NMR (400 MHZ, CDCl₃) ô: 6.48 (s, 2H), 4.24 (s, 2H), 3.95 (dt, J=11.0, 6.6 Hz, 6H), 1.83-1.70
(m, 6H), 1.46 (q, J=7.3 Hz, 6H), 1.27 (d, J=6.9 Hz, 48H), 0.88 (t, J=6.8 Hz, 9H).

¹³C NMR (100 MHZ, CDCl₃) δ : 153.50, 138.28, 130.50, 106.77, 77.36, 73.58, 69.33, 55.34,
32.10, 32.08, 30.48, 29.91, 29.89, 29.85, 29.81, 29.79, 29.76, 29.57, 29.54, 29.52,
26.27, 26.24,
22.85, 14.27.

MALDI-TOF MS (M+Na): 708.11 g/mol.

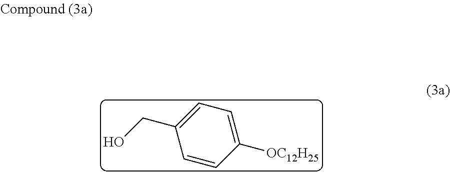

Mol. formula: C₁₉H₃₂O₂
Mol.weight: 292.46 g/mol
Physical appearance: white solid
Yield: 80%

The compound (3a) was prepared from 4-hydroxybezyl alcohol (15 g, 120.87 mmol), 1-bromododecane (33.13 g, 132.95 mmol), K₂CO₃ (22.04 g, 159.54 mmol), 18-crown-6 (3.1 g, 12 mmol) in acetone. The product was obtained as a white solid (28 g, 96 mmol, 80%) after purification by NPC using EtOAc/PE as eluent, R_{f} value=0.71 in 50% EtOAc/PE.

¹H NMR (400 MHZ, CDCl₃) δ : 7.35-7.17 (m, 2H), 6.92-6.82 (m, 2H), 4.58 (d, J=3.6 Hz,
2H), 3.94 (t, J=6.6 Hz, 2H), 1.77 (dt, J=14.8, 6.5 Hz, 3H), 1.52-1.39 (m, 2H), 1.39-1.18 (m, 16H), 0.94-0.81 (m, 3H).

¹³C NMR (100 MHZ, CDCl₃) δ : 158.91, 133.02, 128.75, 114.68, 68.20, 65.17, 32.05, 29.79,
29.77, 29.73, 29.71, 29.62, 29.53, 29.48, 29.39, 26.17, 22.82, 14.24.

MALDI-TOF MS (M+K): 331.22 g/mol.

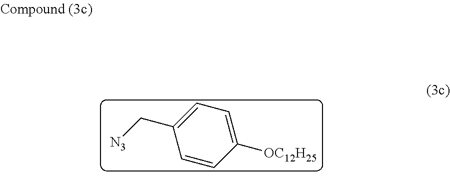

Mol. formula: C₁₉H₃₁O₃N₃
Mol. weight: 317.48 g/mol
Physical appearance: White
solid Yield: 85%
   The compound (3b), a chloride was achieved using compound (3a) (23 g, 77.7 mmol), SOCl₂ (14.6 g, 124.3 mmol), and a catalytic amount of DMF in DCM. Subsequently, azide, compound (3c) was synthesized using NaN₃ (10.10 g, 155.4 mmol) in DMF from the compound (3b) without purification. The product was obtained as a white solid (20.9 g, 66.0 mmol, 85%) after purification by NPC using EtOAc/PE as eluent, R_{f} value=0.73 in 10% EtOAc/PE.

¹H NMR (400 MHZ, CDCl₃) δ : 7.28-7.22 (m, 2H), 6.94-6.88 (m, 2H), 4.27 (s, 2H), 3.97 (t, J=6.6 Hz, 2H), 1.85-1.75 (m, 2H), 1.53-1.43 (m, 2H), 1.41-1.24 (m, 16H), 0.91 (t, J=6.8 Hz, 3H).

¹³C NMR (100 MHZ, CDCl₃) δ : 159.34, 129.94, 129.80, 129.53, 127.53, 127.25, 126.89, 114.84, 114.52, 114.49, 114.47, 114.40, 68.15, 54.62, 54.53, 32.05, 31.61, 31.53, 30.30, 30.25, 29.83, 29.79, 29.77, 29.73, 29.71, 29.52, 29.48, 29.37, 28.72, 26.16, 22.82, 22.46, 14.23, 14.17.

MALDI-TOF MS (M+K): 356.22 g/mol

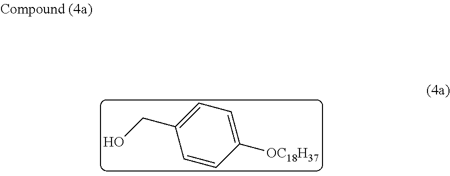

Mol. formula: C₂₅H₄₄O₂
Mol.weight: 376.33 g/mol
Physical appearance: white solid
Yield: 80%
   The compound (4a) was prepared from 4-hydroxymethyl alcohol (15 g, 120 mmol), 1-bromododecane (45 g, 133 mmol), K₂CO₃ (25.5 g, 144 mmol), 18-crown-6 (3.19 g, 12 mmol) in acetone. The product was obtained as a white solid (36.12 g,96 mmol, 80%) after purification by NPC using EtOAc/PE as eluent, R_{f} value=0.72 in 50% EtOAc/PE.

¹H NMR (400 MHZ, CDCl₃) δ : 7.30-7.25 (m, 2H), 6.91-6.85 (m, 2H), 4.61 (s, 2H), 3.95 (t,
J=6.6 Hz, 2H), 1.77 (dt, J=14.7, 6.8 Hz, 2H), 1.49-1.40 (m, 2H), 1.26 (s, 28H), 0.88 (t, J=
6.8 Hz, 3H).

¹³C NMR: (100 MHz, CDCl₃) δ : 158.96, 133.02, 128.77, 114.71, 68.22, 65.26, 32.07, 29.85,
29.81, 29.75, 29.73, 29.55, 29.51, 29.41, 26.19, 22.84, 14.26.

MALDI-TOF MS (M+K): 415.31 g/mol.

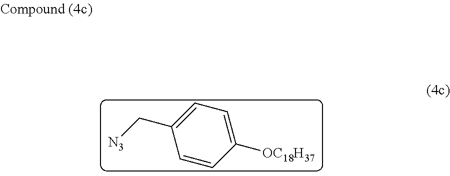

Mol. formula: C₂₅H₄₃N₃O
Mol. weight: 401.64 g/mol
Physical appearance: Whitesolid
Yield: 82%
   The compound (4b), a chloride was achieved starting from compound (4a) (36.12 g, 96 mmol), SOCl₂ (18.12 g, 153.6 mmol), and a catalytic amount of DMF in DCM. Subsequently, compound (4b), an azide, was synthesized using NaN₃ (7.5 g, 115.5 mmol) and tetra-n-butylammonium bromide (TBAB) (37.22 g, 115.5 mmol) in tetrahydrofuran (THF) and dimethylformamide (DMF) (1:1 ratio). The product was obtained as a white solid (31.61 g, 78.7 mmol, 82%) after purification by NPC using EtOAc/PE as eluent, R_{f} value=0.44 in 5% EtOAc/PE.

¹H NMR (400 MHZ, CDCl₃) δ : 7.23 (d, J=8.6 Hz, 2H), 6.93-6.87 (m, 2H), 4.26 (s, 2H), 3.96 (t, J=6.6 Hz, 2H), 1.79 (dt, J=14.7, 6.7 Hz, 2H), 1.49-1.42 (m, 2H), 1.27 (s, 28H), 0.93-0.85 (m, 3H).

¹³C NMR (100 MHz, CDCl₃) δ : 159.37, 129.84, 127.27, 114.88, 68.20, 54.58, 32.08, 29.85, 29.81, 29.75, 29.72, 29.55, 29.51, 29.39, 26.19, 22.84, 14.26.

MALDI-TOF MS (M+Na): 424.35 g/mol.

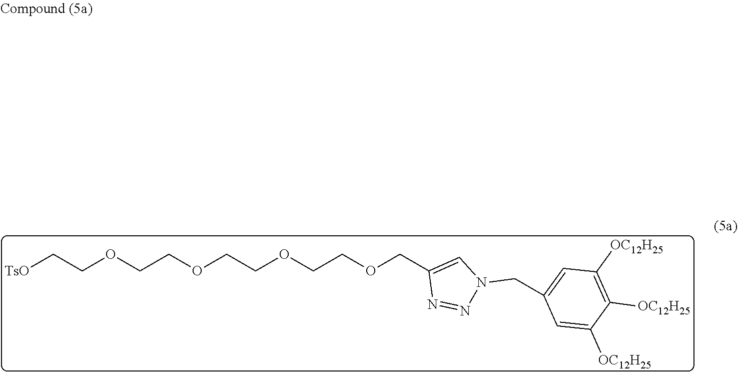

Mol. formula: C₆₁H₁₀₅N₃O₁₀S Mol. weight: 1071.75 g/mol.
Physical appearance: yellow waxy solid
Yield: 85%
   Alkyne (1b) (0.250 g, 0.646 mmol) and azide (2e) (3.5 g, 2.58 mmol) were weighed in an oven-dried RBF, and THF (7 ml) was added, followed by water (7 ml) with vigorous stirring. Then CuSO₄ (16 mg, 0.06 mmol), Na ascorbate (6 mg, 0.032 mmol) solution added and allowed to react overnight. The reaction was extracted with DCM and then concentrated under reduced pressure. The resulting crude material was purified by NPC using MeOH/DCM (0.600 g, 0.55 mmol, 85%), R_{f} value=0.33 in 10% MeOH/DCM

¹H NMR (400 MHZ, CDCl₃) δ : 7.81-7.75 (m, 2H), 7.54-7.47 (m, 1H), 7.33 (d, J=8.0 Hz, 2H), 6.45 (s, 2H), 5.37 (s, 2H), 4.65 (s, 2H), 4.22-4.11 (m, 2H), 3.91 (q, J=6.3 Hz, 6H), 3.69-3.55 (m, 14H), 2.43 (s, 3H), 1.78-1.71 (m, 7H), 1.48-1.40 (m, 6H), 1.25 (s, 48H), 0.87 (t, J=6.7 Hz, 9H).

¹³C NMR (100 MHZ, CDCl₃) δ : 153.69, 144.93, 138.58, 133.11, 129.96, 129.52, 128.10, 106.89, 73.59, 70.85, 70.71, 70.66, 70.62, 69.89, 69.37, 68.80, 64.84, 54.66, 32.06, 30.45, 29.88, 29.86, 29.83, 29.79, 29.77, 29.73, 29.55, 29.52, 29.50, 26.24, 26.21, 22.82, 21.76, 14.24.

MALDI-TOF MS (M+Na): 1095.22 g/mol.

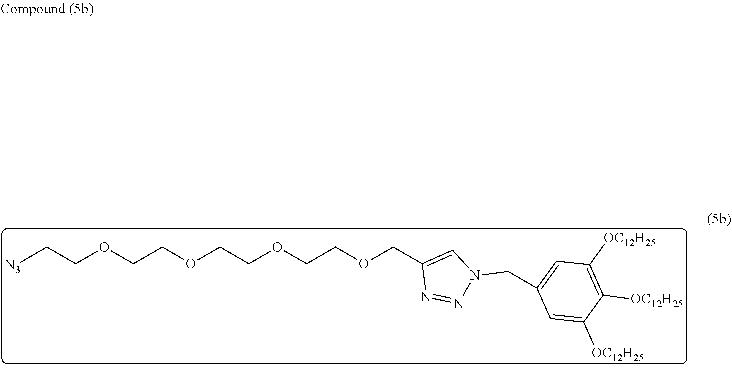

Mol. formula: C₅₄H₉₈N₆O₇
Mol. weight: 943.41 g/mol.
Physical appearance: yellow waxy
solid Yield: 76%
   Tosylate, compound (5a) (0.600 g, 0.559 mmol) was dissolved in DMF (30 ml) at RT. Then NaN₃ (0.072 g, 1.11 mmol) was added to the reaction mixture and allowed to react at RT for 12 hrs. After this, the reaction mixture was concentrated and purified by NPC without any workup (0.420 g, 76%), R_{f} value=0.40 in 10% MeOH/DCM

¹H NMR (400 MHZ, CDCl₃) δ : 7.48 (s, 1H), 6.45 (s, 2H), 5.37 (s, 2H), 4.66 (s, 2H), 3.91 (q, J=6.3 Hz, 6H), 3.72-3.58 (m, 14H), 3.37 (t, J=5.1 Hz, 2H), 1.80-1.69 (m, 6H), 1.45 (dq, J=8.4, 4.8 Hz, 6H), 1.33-1.25 (m, 49H), 0.89-0.85 (m, 9H).

¹³C NMR (100 MHZ, CDCl₃) δ : 153.71, 145.60, 138.61, 129.48, 122.55, 106.90, 77.36, 73.60, 70.80, 70.77, 70.74, 70.68, 70.62, 70.15, 69.90, 69.38, 64.85, 54.61, 50.80, 32.07, 32.05, 30.45, 29.88, 29.86, 29.83, 29.82, 29.79, 29.77, 29.73, 29.55, 29.52, 29.50, 26.24, 26.21, 22.82, 14.24.

MALDI-TOF MS (M+Na): 966.04 g/mol.

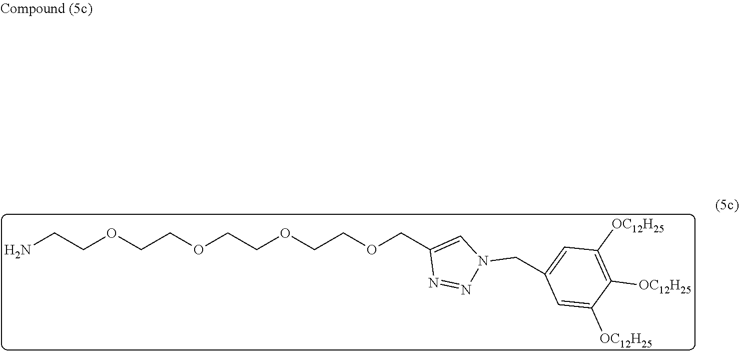

Mol. formula: C₅₄H₁₀₀N₄O₇ Mol. weight: 916.74 g/mol.
Physical appearance: yellow waxy solid Yield: 55%
   Azide, compound (5b) (0.110 gm, 0.116 mmol) was dissolved in THF (10 ml) at 40° C. and PPh₃ (0.112 gm, 0.466 mmol) dissolved in THF (5 ml) and was added at 40° C. 1M NaOH (15 ml) was added and maintained the pH of the solution at 10-11. Then the reaction mixture was allowed to react at 40° C. for 16 hrs. After completion of the reaction, RM was neutralized by acidic water. Then the reaction mixture was extracted with DCM and purified using NPC (0.058 gm, 0.063 mmol, 55%), R_{f} value=0.40 in 10% MeOH/DCM.

¹H NMR (400 MHZ, CDCl₃) δ: 7.56 (s, 1H), 6.48 (s, 2H), 5.39 (s, 2H), 4.64 (s, 2H), 3.91 (t, J=6.5 Hz, 6H), 3.69 (d, J=1.3 Hz, 2H), 3.65-3.57 (m, 12H), 2.84 (t, J=5.0 Hz, 2H), 1.79-1.69 (m, 6H), 1.47-1.40 (m, 6H), 1.25 (s, 48H), 0.87 (t, J=6.8 Hz, 9H).

¹³C NMR (100 MHZ, CDCl₃) δ : 153.68, 144.95, 138.55, 129.45, 122.79, 106.95, 73.58, 71.00, 70.48, 70.39, 70.37, 70.20, 70.02, 69.36, 64.67, 54.66, 41.06, 32.06, 32.05, 30.44, 29.87, 29.85, 29.83, 29.82, 29.78, 29.76, 29.73, 29.63, 29.55, 29.51, 29.49, 26.23, 26.22, 22.81, 14.24.

MALDI-TOF MS (M+Na): 940.03 g/mol.

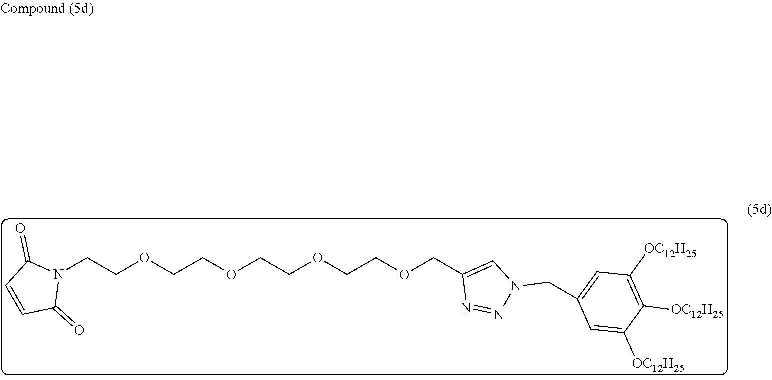

Mol. formula: C₅₈H₁₀₀N₄O₉
Mol. weight: 997.46 g/mol.
Physical appearance: yellow oil brownish oil
Yield: 45%
   Amine, compound (5c) (2.1 gm, 2.29 mmol) was dissolved in a mixture of saturated aqueous solution of NaHCO₃ (10 ml) and THF (10 ml) and cooled in an ice bath. Then N-(methoxycarbonyl) maleimide (0.424 gm, 2.74 mmol) was added in portions over 5 mins under vigorous stirring. The mixture was stirred for 2 hr at 0° C., followed by 6 hr at 30° C. After extraction with DCM, the organic phase was dried over anhydrous Na₂SO₄, filtered, and concentrated. Purification by NPC using MeOH/DCM yielded the product (5d) as a brownish oil. (1.02 gm, 1.03 mmol 45%), R_{f} value=0.35 in 5% MeOH/DCM

¹H NMR (400 MHZ, CDCl₃) δ : 7.49 (s, 1H), 6.68 (s, 2H), 6.46 (s, 2H), 5.38 (s, 2H), 4.66 (s, 2H), 3.94-3.88 (m, 6H), 3.71-3.56 (m, 16H), 1.81-1.76 (m, 4H), 1.71 (d, J=7.9 Hz, 2H), 1.47-1.41 (m, 6H), 1.25 (d, J=2.4 Hz, 48H), 0.87 (d, J=7.0 Hz, 9H).

¹³C NMR (100 MHZ, CDCl₃) δ : 170.79, 153.72, 145.61, 138.63, 134.28, 129.46, 122.63, 106.93, 77.37, 73.61, 70.72, 70.66, 70.61, 70.17, 69.92, 69.39, 67.96, 64.81, 54.66, 37.26, 32.07, 30.46, 29.89, 29.88, 29.84, 29.80, 29.78, 29.75, 29.65, 29.57, 29.53, 29.51, 26.25, 26.23, 22.83, 14.26.

MALDI-TOF MS (M+Na): 1019.46 g/mol.

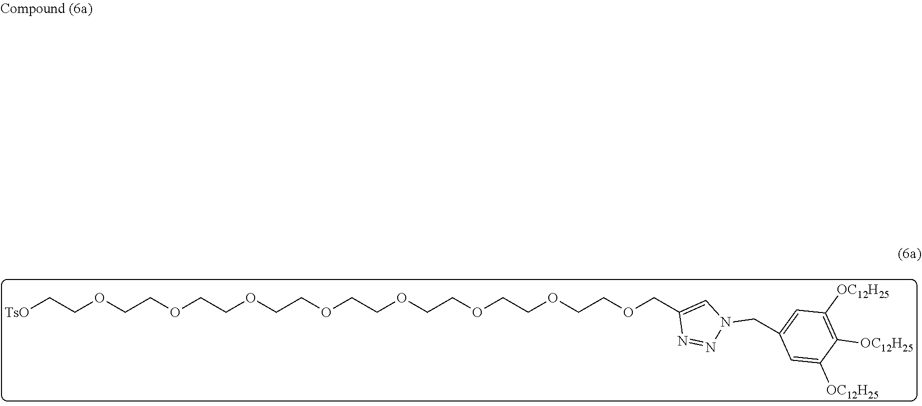

Mol. formula: C₆₉H₁₂1N₃O₁₄S Mol. weight: 1248.79 g/mol.
Physical appearance: yellow waxy solid
Yield: 85%
   Alkyne (1d) (1.63 g, 2.91 mmol) and azide (2e) (7.98 g, 11.6 mmol) were weighed in an oven-dried RBF, and THF (7 ml) was added, followed by water (7 ml) with vigorous stirring. Then CuSO₄ (72 mg, 0.291 mmol), Na ascorbate (28 mg, 0.145 mmol) and allowed to react overnight. The reaction was extracted with DCM and then concentrated under reduced pressure. The resulting crude material was purified by NPC using MeOH/DCM (3.08 g, 2.47 mmol, 85%), R_{f} value=0.33 in 10% MeOH/DCM

¹H NMR (400 MHZ, CDCl₃) δ : 7.81-7.77 (m, 2H), 7.54 (s, 1H), 7.34 (d, J=8.1 Hz, 2H), 6.45 (s, 2H), 5.38 (s, 2H), 4.65 (s, 2H), 4.17-4.13 (m, 2H), 3.91 (h, J=5.0 Hz, 6H), 3.69-3.57 (m, 30H), 2.44 (s, 3H), 1.80-1.75 (m, 4H), 1.69 (d, J=6.7 Hz, 2H), 1.43 (q, J=6.7 Hz, 6H), 1.27 (d, J=7.7 Hz, 48H), 0.87 (t, J=6.7 Hz, 9H).

¹³C NMR (100 MHZ, CDCl₃) δ : 153.71, 144.93, 138.64, 133.15, 129.96, 129.41, 128.12, 106.93, 77.36, 73.61, 70.87, 70.73, 70.67, 70.64, 69.93, 69.39, 68.82, 32.06, 30.46, 29.89, 29.87, 29.84, 29.80, 29.78, 29.74, 29.56, 29.53, 29.51, 26.25, 26.22, 22.83, 21.78, 14.25.

MALDI-TOF MS (M+K): 1287.06 g/mol.

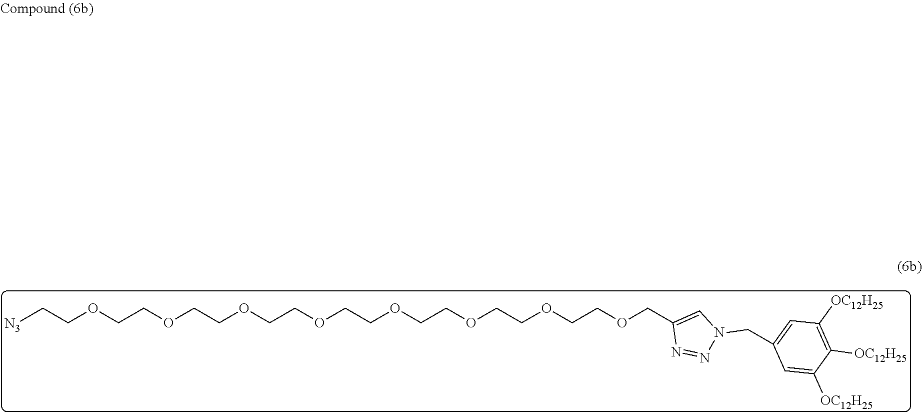

Mol. formula: C₆₂H₁₁₄N₆O₁₁ Mol. weight: 1119.63 g/mol.
Physical appearance: yellow waxy
solid Yield: 45%
   Tosylate (compound 6a) (6 g, 4.80 mmol) was dissolved in DMF (30 ml) at RT. Then NaN₃ (0.62 g, 9.6 mmol) was added to the reaction mixture and allowed to react at RT for 12 hrs. After this, the reaction mixture was concentrated and purified by NPC (2.4 g, 2.16 mmol, 45%),
R_{f} value=0.42 in 10% MeOH/DCM

¹H NMR (400 MHZ, CDCl₃) ô: 7.48 (s, 1H), 6.44 (s, 2H), 5.37 (s, 2H), 4.65 (s, 2H), 3.93-3.85 (m, 6H), 3.68-3.60 (m, 30H), 3.37 (t, J=5.1 Hz, 2H), 1.80-1.68 (m, 6H), 1.43 (tt, J=12.1, 4.6 Hz, 6H), 1.26 (d, J=16.1 Hz, 49H), 0.86 (t, J=6.8 Hz, 9H).

¹³C NMR (400 MHZ, CDCl₃) δ : 153.66, 145.55, 138.56, 129.48, 122.58, 106.85, 73.55, 70.78, 70.75, 70.71, 70.64, 70.62, 70.57, 70.12, 69.85, 69.33, 64.80, 54.57, 50.77, 32.03, 32.02, 30.41, 29.85, 29.83, 29.80, 29.79, 29.76, 29.73, 29.70, 29.51, 29.48, 29.46, 26.25, 26.21, 26.18, 22.78, 14.21.

MALDI-TOF MS (M+Na): 1142.14 g/mol.

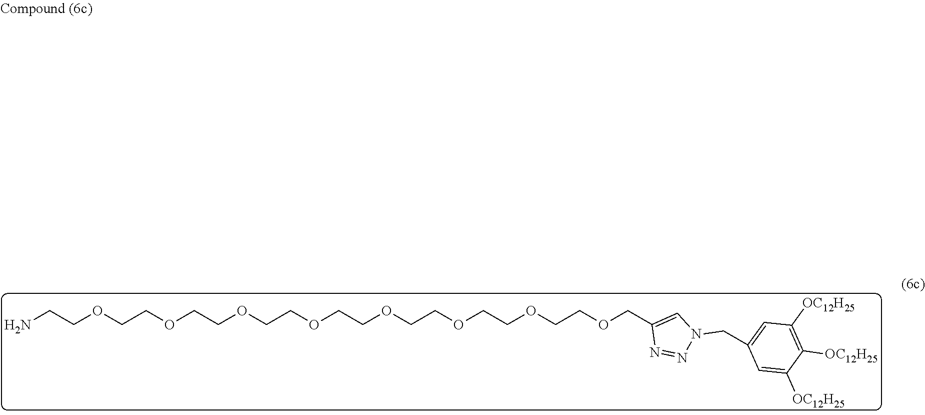

Mol. formula: C₆₂H₁₁₆N₄O₁₁
Mol. weight: 1093.63 g/mol.
Physical appearance: yellow waxy solid
Yield: 51%
   Azide compound (6b) (2.41 g, 2.14 mmol) was dissolved in THF (10 ml) at 40° C. and PPh₃ (2.24 g, 8.56 mmol) dissolved in THF (5 ml) and was added at 40° C. 1M NaOH (15 ml) was added and maintained the pH of the solution at 10-11. Then the reaction mixture was allowed to react at 40° C. for 16 hrs. After completion of the reaction, RM was neutralized by acidic water. Then the reaction mixture was extracted with DCM and purified using NPC (1.34 g, 1.22 mmol, 51%), R_{f} value=0.29 in 10% MeOH/DCM

¹H NMR (400 MHZ, CDCl₃) ô: 7.49 (s, 1H), 6.44 (s, 2H), 5.36 (s, 2H), 4.64 (s, 2H), 3.92-3.87 (m, 6H), 3.67-3.59 (m, 29H), 3.50 (t, J=5.2 Hz, 2H), 2.86 (q, J=4.2 Hz, 1H), 1.79-1.67 (m, 6H), 1.42 (dq, J=11.2, 7.0 Hz, 6H), 1.25 (d, J=6.6 Hz, 49H), 0.86 (t, J=6.7 Hz, 9H).

¹³C NMR (100 MHZ, CDCl₃): 153.65, 145.51, 138.54, 129.51, 122.61, 106.84, 73.55, 73.05, 70.63, 70.61, 70.55, 70.33, 69.83, 69.33, 64.79, 54.55, 41.75, 32.02, 30.41, 29.84, 29.82, 29.79, 29.75, 29.73, 29.69, 29.51, 29.48, 29.46, 26.20, 26.18, 22.78, 14.21.

MALDI-TOF MS (M+Na): 1116.07 g/mol.

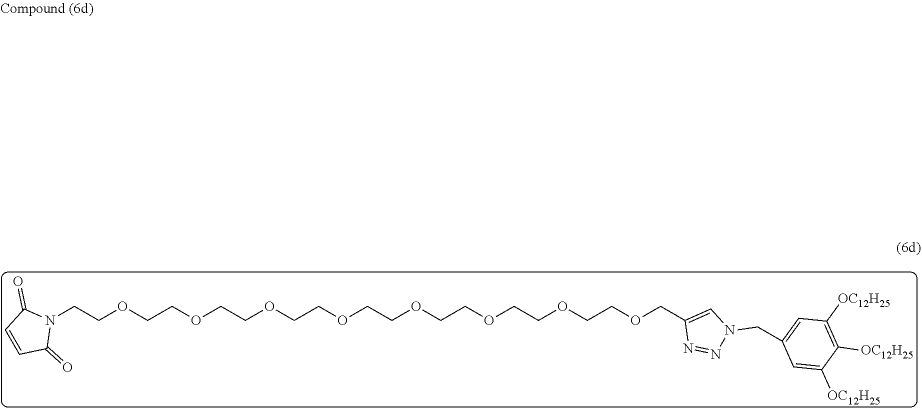

Mol. formula: C₆₆H₁₁₆N₄O₁₃ Mol. weight: 1173.67 g/mol. Physical appearance: yellow oil Yield: 45%
   Amine, compound (6d) (2.34 g, 2.14 mmol) was dissolved in a mixture of saturated aqueous solution of NaHCO₃ (10 ml) and THF (10 ml) and cooled in an ice bath. Then N-(methoxycarbonyl) maleimide (0.398 g, 2.56 mmol) was added in portions over 5 mins under vigorous stirring. The mixture was stirred for 2 hr at 0° C., followed by 6 hr at 30° C. After extraction with DCM, the organic phase was dried over anhydrous Na₂SO₄, filtered, and concentrated. Purification by NPC using MeOH/DCM yielded the product as a yellow oil. (1.12 g, 0.96 mmol 45%), R_{f} value=0.33 in 10% MeOH/DCM

¹H NMR (400 MHZ, CDCl₃) δ : 7.49 (s, 1H), 6.68 (s, 2H), 6.44 (s, 2H), 5.36 (s, 2H), 4.64 (s, 2H), 3.89 (h, J=5.3 Hz, 6H), 3.73-3.53 (m, 32H), 1.73 (dq, J=18.8, 7.1 Hz, 6H), 1.42 (dq, J=11.4, 6.7 Hz, 6H), 1.24 (s, 48H), 0.85 (t, J=6.6 Hz, 9H).

¹³C NMR (100 MHZ, CDCl₃) δ : 170.72, 153.63, 145.53, 138.53, 134.23, 129.40, 122.60,
106.84, 73.51, 70.66, 70.60, 70.58, 70.52, 70.10, 69.82, 69.30, 67.87, 64.70, 54.57, 37.18,
31.99, 30.38, 29.81, 29.79, 29.76, 29.72, 29.70, 29.66, 29.52, 29.48, 29.45, 29.43, 26.22, 26.18,
26.15, 22.75, 14.18

MALDI-TOF MS (M+K): 1212.09 g/mol.

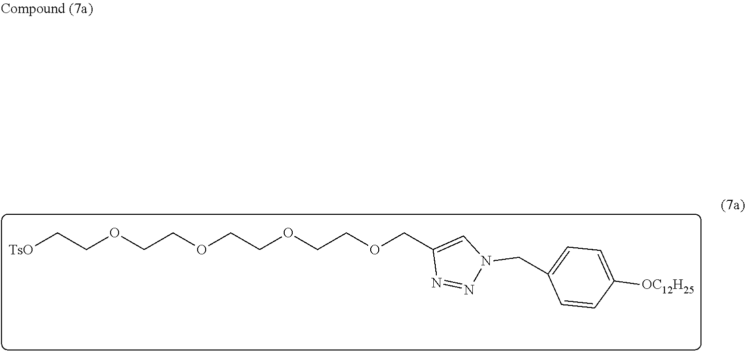

Mol. formula: C₃₇H₅₇N₃O₈S Mol. weight: 703.94 g/mol.
Physical appearance: Yellowish waxy solid Yield: 75%
   Alkyne (1d) (2.31 g, 5.98 mmol) and azide (3c) (7.8 g, 23.6 mmol) were weighed in an oven-dried RBF, and THF (7 ml) was added, followed by water (7 ml) with vigorous stirring. Then CuSO₄ (15 mg, 0.59 mmol), Na ascorbate (59 mg, 0.299 mmol) was added and allowed to react overnight. The reaction was extracted with DCM and then concentrated under reduced pressure. The resulting crude material was purified by NPC using MeOH/DCM (3.1 g, 4.48 mmol, 75%), R_{f} value=0.35 in 10% MeOH/DCM

¹H NMR (400 MHZ, CDCl₃) δ 7.80-7.69 (m, 2H), 7.55-7.46 (m, 1H), 7.29 (d, J=8.1 Hz, 2H), 7.17 (d, J=8.3 Hz, 2H), 6.90-6.73 (m, 2H), 5.39 (s, 2H), 4.60 (s, 2H), 4.20-4.03 (m, 2H), 3.89 (t, J=6.6 Hz, 2H), 3.71-3.48 (m, 14H), 2.40 (s, 3H), 1.80-1.65 (m, 2H), 1.41 (dd, J=10.1, 5.4 Hz, 2H), 1.33-1.21 (m, 17H), 0.84 (t, J=6.8 Hz, 3H).

¹³C NMR (100 MHz, CDCl₃) δ : 159.46, 144.80, 132.95, 129.82, 129.67, 129.39, 127.93,
126.33, 114.94, 114.27, 70.68, 70.54, 70.48, 70.45, 69.68, 69.27, 68.62, 68.08, 64.68, 53.75,
31.88, 31.48, 30.14, 29.66, 29.62, 29.60, 29.56, 29.54, 29.35, 29.31, 29.17, 25.99, 22.66, 22.30,
21.61, 14.11.

MALDI-TOF MS (M+K): 742.53 g/mol.

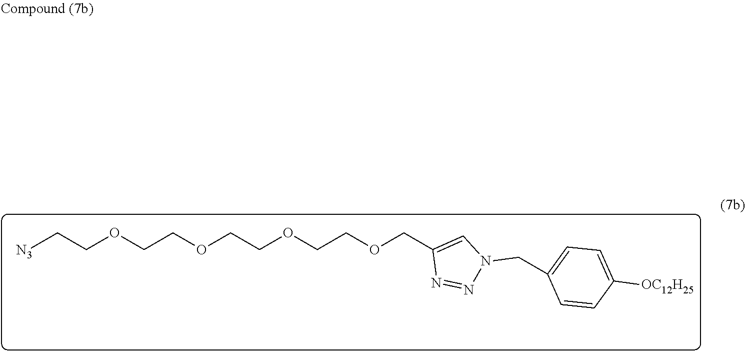

Mol. formula: C₃₀H₅₀N₆O₅ Mol. weight: 574.38 g/mol.
Physical appearance: yellow waxy solid Yield: 70%
   Tosylate, compound (7a) (3.1 g, 4.48 mmol) was dissolved in DMF (40 ml) at RT. Then NaN₃ (0.582 g, 8.96 mmol) was added to the reaction mixture and allowed to react at RT for 12 hrs. After this, the reaction mixture was concentrated and purified by NPC (1.8 g, 3.13 mmol, 70%),
R_{f} value=0.42 in 10% MeOH/DCM

¹H NMR (400 MHZ, CDCl₃) δ: 7.43 (s, 1H), 7.23-7.13 (m, 2H), 6.89-6.82 (m, 2H), 5.41 (s,
2H), 4.62 (s, 2H), 3.92 (t, J=6.6 Hz, 2H), 3.69-3.58 (m, 14H), 3.35 (t, J=5.0 Hz, 2H), 1.82-1.68
(m, 2H), 1.47-1.19 (m, 22H), 0.86 (t, J=6.7 Hz, 4H).

¹³C NMR (100 MHZ, CDCl₃) δ: 159.57, 145.51, 130.05, 129.88, 129.75, 128.02, 126.35, 122.31, 115.32, 115.05, 114.37, 70.73, 70.70, 70.67, 70.61, 70.55, 70.07, 69.78, 69.32, 68.72,
68.17, 64.76, 53.77, 50.73, 31.96, 31.56, 31.48, 30.20, 29.75, 29.71, 29.68, 29.64, 29.62, 29.43,
29.40, 29.25, 26.07, 22.74, 14.18.

MALDI-TOF MS (M+K): 613.4 g/mol.

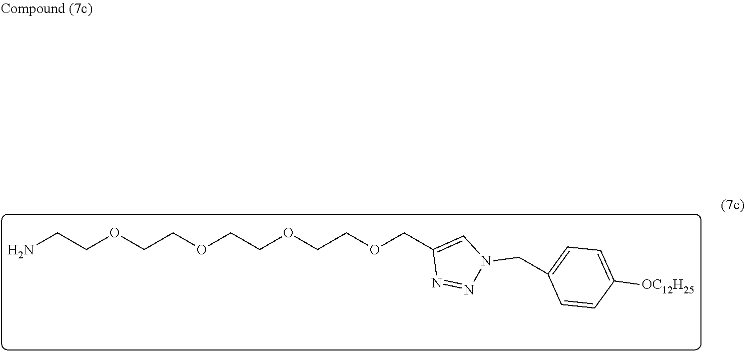

Mol. formula: C₃₀H₅₂N₄O₅ Mol. weight: 548.77 g/mol.
Physical appearance: yellow waxy solid Yield: 55%
   Azide, compound (7b) (1.8 g, 3.13 mmol) was dissolved in THF (10 ml) at 40° C. and PPh₃ (3.2 g, 12.52 mmol) dissolved in THF (5 ml) and was added at 40° C. 1M NaOH (15 ml) was added and the pH of the solution was maintained at 10-11. Then the reaction mixture was allowed to react at 40° C. for 16 hrs. After completion of the reaction, RM was neutralized by acidic water. Then the reaction mixture was extracted with DCM and purified using NPC (0.943 g, 1.72 mmol, 55%), R_{f} value=0.25 in 10% MeOH/DCM

¹H NMR (400 MHZ, CDCl₃) δ : 7.58 (s, 1H), 7.25-7.21 (m, 2H), 6.88-6.83 (m, 2H), 5.44 (s,
2H), 4.63 (s, 2H), 3.92 (t, J=6.6 Hz, 2H), 3.75-3.57 (m, 14H), 2.89-2.78 (m, 3H), 1.79-1.70
(m, 2H), 1.42 (tdd, J=8.7, 4.4, 2.3 Hz, 3H), 1.37-1.25 (m, 17H), 0.87 (s, 3H)

¹³C NMR (100 MHz, CDCl₃) δ : 159.64, 144.77, 129.94, 126.38, 124.48, 123.58, 122.69,
119.14, 118.96, 115.10, 70.40, 70.31, 70.28, 70.13, 70.00, 68.25, 64.57, 53.91, 46.03, 40.76,
35.09, 34.55, 32.03, 31.62, 31.55, 30.30, 30.25, 29.81, 29.77, 29.74, 29.71, 29.68, 29.50, 29.46,
29.31, 26.14, 22.80, 14.23, 10.04, 8.04.

MALDI-TOF MS (M+Na): 571.5 g/mol.

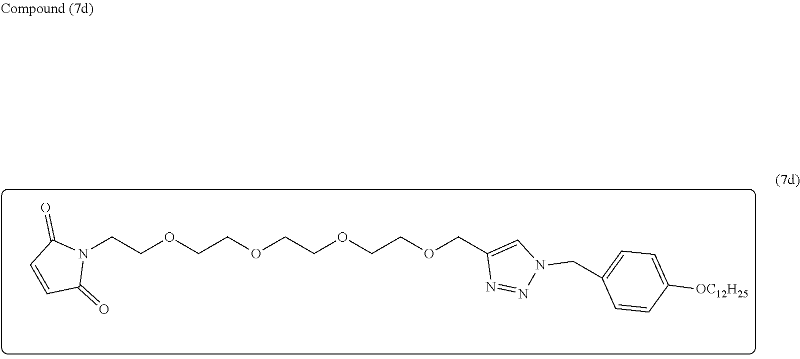

Mol. formula: C₃₄H₅₂N₄O₇
Mol. weight: 628.81 g/mol.
Physical appearance: yellow
oil Yield: 45%
   Amine, compound (7c) (0.943 g, 1.72 mmol) was dissolved in a mixture of saturated aqueous solution of NaHCO₃ (10 ml) and THF (10 ml) and cooled in an ice bath. Then N-(methoxycarbonyl) maleimide (1.28 g, 8.2 mmol) was added in portions over 5 mins under vigorous stirring. The mixture was stirred for 2 hr at 0° C., followed by 6 hr at 30° C. After extraction with DCM, the organic phase was dried over anhydrous Na₂SO₄, filtered, and concentrated. Purification by NPC using MeOH/DCM yielded the product as a yellow oil, (0.486 g, 0.774 mmol, 45%), R_{f} value=0.36 in 10% MeOH/DCM

¹H NMR (400 MHZ, CDCl₃) δ: 7.46 (s, 1H), 7.19 (d, J=8.4 Hz, 2H), 6.85 (d, J=8.4 Hz, 2H), 6.66 (s, 2H), 5.41 (s, 2H), 4.62 (s, 2H), 3.91 (t, J=6.5 Hz, 2H), 3.68 (q, J=7.1, 6.5 Hz, 2H), 3.64-3.55 (m, 12H), 1.79-1.70 (m, 2H), 1.44-1.38 (m, 3H), 1.36-1.24 (m, 16H), 0.85 (t, J=6.6 Hz, 4H)

¹³C NMR (100 MHZ, CDCl₃) δ : 170.74, 159.57, 145.46, 139.32, 134.21, 129.76, 126.37, 122.44, 115.94, 115.05, 114.14, 70.63, 70.56, 70.51, 70.09, 69.77, 68.18, 67.88, 64.71, 53.79, 53.52, 52.31, 37.19, 34.84, 33.88, 31.99, 31.98, 31.72, 31.57, 30.36, 30.20, 29.76, 29.72, 29.69, 29.66, 29.63, 29.57, 29.55, 29.45, 29.42, 29.41, 29.26, 29.22, 29.01, 26.08, 25.67, 24.95, 22.75, 14.19.

MALDI-TOF MS (M+Na): 651.51 g/mol.

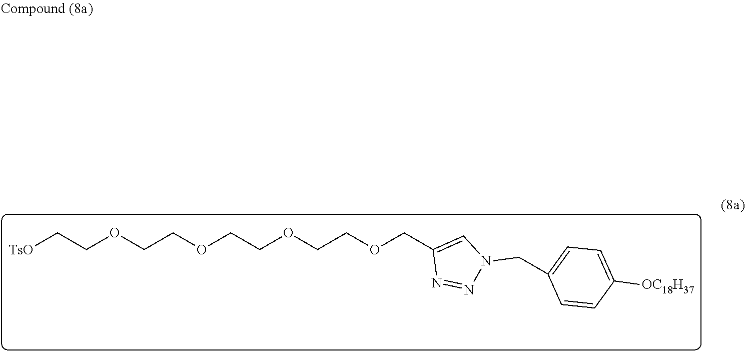

Mol. formula: C₄₃H₆₉N₃O₈S Mol. weight: 788.10 g/mol.
Physical appearance: Waxy white solid
Yield: 85%
   Alkyne (1b) (1.89 g, 4.89 mmol) and azide (4c) (7.8 g, 19.56 mmol) were weighed in an oven-dried RBF, and THF (7 ml) was added, followed by water (7 ml) with vigorous stirring. CuSO₄ (123 mg, 0.48 mmol), Na ascorbate (48 mg, 0.244 mmol) was added and allowed to react overnight. The reaction was extracted with DCM and then concentrated under reduced pressure. The resulting crude material was purified by NPC using MeOH/DCM (3.27 g, 4.15 mmol, 85%), R_{f} value=0.34 in 10% MeOH/DCM

¹H NMR (400 MHZ, CDCl₃) δ : 7.76-7.68 (m, 2H), 7.44 (s, 1H), 7.29 (d, J=8.1 Hz, 2H),
7.21-7.07 (m, 2H), 6.88-6.78 (m, 2H), 5.39 (s, 2H), 4.60 (s, 2H), 4.16-4.06 (m, 2H), 3.89
(t, J=6.6 Hz, 2H), 3.67-3.47 (m, 14H), 2.40 (s, 3H), 1.77-1.68 (m, 2H), 1.44-1.36 (m, 2H), 1.28 (s, 29H), 0.84 (t, J=6.7 Hz, 3H).

¹³C NMR (100 MHZ, CDCl₃) δ : 166.06, 151.99, 151.39, 139.58, 137.51, 136.43, 136.27,
136.00, 135.41, 134.54, 132.95, 128.97, 121.80, 121.54, 84.08, 83.76, 83.44, 77.29, 77.16,
77.10, 77.07, 77.06, 76.29, 75.88, 75.23, 74.68, 72.12, 71.49, 71.25, 60.26, 38.51, 38.09, 38.02,
37.15, 36.91, 36.28, 29.27, 28.21, 20.72, 20.32.

MALDI-TOF MS (M+Na): 811.6 g/mol.

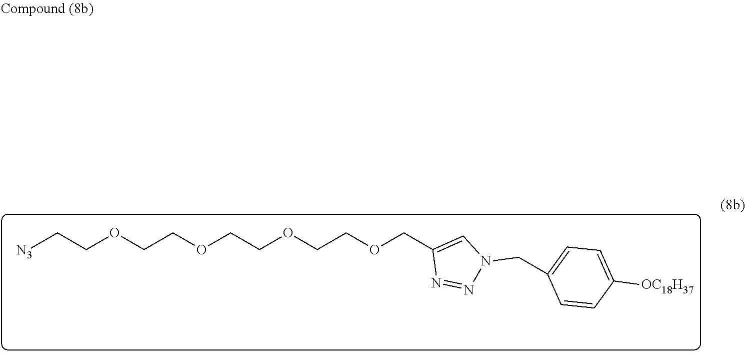

Mol. formula: C₃₆H₆₂N₆O₅
Mol. weight: 658.93 g/mol.
Physical appearance: Waxy white solid
Yield: 55%
   Tosylate, compound (8a) (3.27 g, 4.15 mmol) was dissolved in DMF (30 ml) at RT. Then NaN₃ (0.539 g, 8.3 mmol) was added to the reaction mixture and allowed to react at RT for 12 hrs. After this, the reaction mixture was concentrated and purified by NPC (1.50 g, 2.28 mmol, 55%), R_{f} value=0.42 in 10% MeOH/DCM

¹H NMR (400 MHZ, CDCl₃) δ : 7.43 (d, J=3.7 Hz, 1H), 7.15 (d, J=8.6 Hz, 2H), 6.87-6.75 (m, 2H), 5.36 (s, 2H), 4.58 (d, J=3.7 Hz, 2H), 3.87 (t, J=6.5 Hz, 2H), 3.67-3.44 (m, 14H), 3.42-3.20 (m, 2H), 1.82-1.61 (m, 2H), 1.39 (dd, J=10.7, 5.1 Hz, 2H), 1.21 (s, 28H), 0.90-0.73 (m, 3H).

¹³C NMR (100 MHz, CDCl₃) δ : 159.31, 159.29, 145.22, 144.60, 132.87, 129.68, 129.50, 127.78, 126.50, 126.29, 126.25, 122.24, 122.19, 114.78, 71.16, 70.53, 70.49, 70.47, 70.44, 70.41, 70.37, 70.32, 69.85, 69.55, 69.15, 68.47, 67.90, 64.51, 64.48, 53.47, 50.48, 31.77, 29.55, 29.51, 29.45, 29.43, 29.24, 29.21, 29.05, 25.88, 22.53, 21.44, 13.98.

MALDI-TOF MS (M+K): 681.34 g/mol.

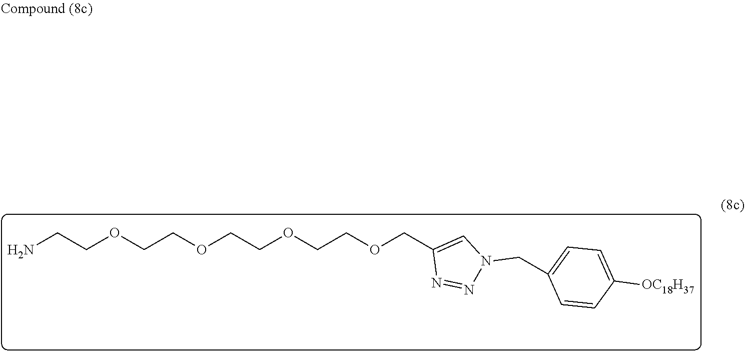

Mol. formula: C₆₂H₁₁₆N₄O₁₁ Mol. weight: 632.93 g/mol.
Physical appearance: Waxy white solid
Yield: 52%
   Azide, compound (8b) (1.50 g, 2.28 mmol) was dissolved in THF (10 ml) at 40° C. and PPh₃ (2.42 g, 9.1 mmol) dissolved in THF (5 ml) and was added at 40° C. 1M NaOH (15 ml) was added and maintained the pH of solution at 10-11. Then the reaction mixture was allowed to react at 40° C. for 16 hrs. After completion of reaction, RM neutralized by acidic water. Then the reaction mixture was extracted with DCM and purified using NPC (0.750 g, 1.18 mmol, 52%), R_{f} value=0.29 in 10% MeOH/DCM

¹H NMR (400 MHZ, CDCl₃) δ : 7.55 (s, 1H), 7.24-7.14 (m, 2H), 6.87-6.78 (m, 2H), 5.40 (s,
2H), 4.59 (s, 2H), 3.88 (t, J=6.6 Hz, 2H), 3.68-3.50 (m, 14H), 2.85 (t, J=5.1 Hz, 2H), 1.78-1.68
(m, 2H), 1.43-1.36 (m, 2H), 1.21 (s, 29H), 0.86-0.81 (m, 3H).

¹³C NMR (100 MHZ, CDCl₃) δ : 159.49, 145.30, 144.92, 129.77, 129.70, 128.72, 126.39,
125.96, 124.37, 123.48, 122.65, 122.44, 114.97, 70.80, 70.48, 70.38, 70.30, 70.04, 69.74,
69.68, 68.12, 64.63, 64.49, 53.72, 40.84, 31.93, 31.51, 30.14, 29.70, 29.67, 29.62, 29.59, 29.41,
29.37, 29.21, 26.34, 26.04, 22.69, 14.14.

MALDI-TOF MS (M+Na): 655.6 g/mol.

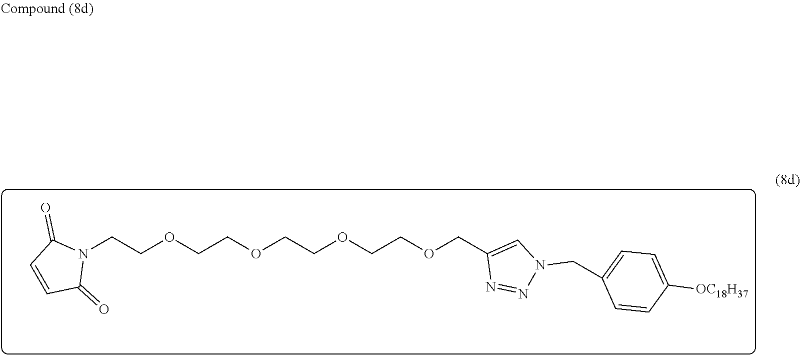

Mol. formula: C₄₀H₆₄N₄O₇ Mol. weight: 712.97 g/mol.
Physical appearance: waxy white solid
Yield: 45%
   Amine, compound (8c) (0.750 g, 1.18 mmol) was dissolved in a mixture of the saturated aqueous solution of NaHCO₃ (10 ml) and THF (10 ml) cooled on an ice bath. Then N-(methoxycarbonyl) maleimide (0.218 g, 1.41 mmol) was added in portions over 5 mins under vigorous stirring. The mixture was stirred for 1 hr at 0° C., followed by 6 hr at 30° C. After extraction with DCM, the organic phase was dried over anhydrous Na₂SO₄, filtered, and concentrated. Purification by NPC using MeOH/DCM, (0.378 g, 0.523 mmol, 45%), R_{f} value=0.33 in 10% MeOH/DCM

¹H NMR (400 MHZ, CDCl₃) δ : 7.55 (s, 1H), 7.24-7.14 (m, 2H), 6.87-6.78 (m, 2H), 5.40
(s, 2H), 4.59 (s, 2H), 3.88 (t, J=6.6 Hz, 2H), 3.68-3.50 (m, 14H), 2.85 (t, J=5.1 Hz, 2H),
1.78-1.68 (m, 2H), 1.43-1.36 (m, 2H), 1.21 (s, 29H), 0.86-0.81 (m, 3H).

¹³C NMR (100 MHz, CDCl₃) δ : 170.57, 159.36, 159.33, 157.65, 145.23, 145.19, 134.04,
129.57, 129.55, 126.32, 122.35, 114.84, 114.82, 114.00, 70.44, 70.36, 70.32, 69.91, 69.58,
67.98, 67.95, 67.68, 64.50, 64.47, 53.54, 53.51, 53.44, 51.96, 51.91, 37.00, 31.81, 31.57, 31.40,
31.32, 29.59, 29.55, 29.50, 29.47, 29.29, 29.25, 29.09, 28.83, 25.92, 22.58, 14.03.

MALDI-TOF MS (M+K): 751.6 g/mol.

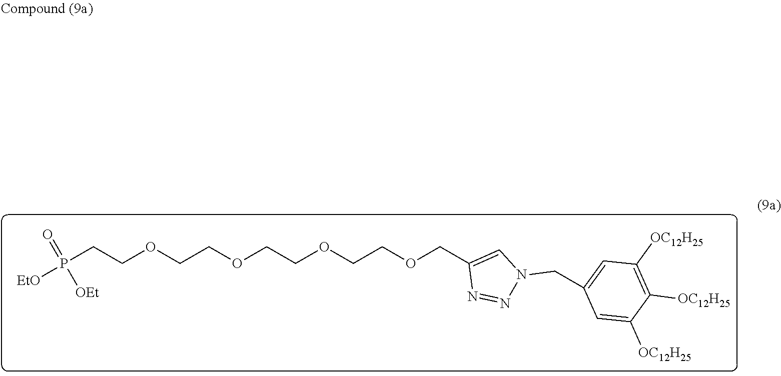

Mol. formula: C₅₈H₁₀₈N₃O₁₀P Mol. weight: 1038.49 g/mol
Physical appearance: brownish liquid
Yield: 75%
   The compound (9a) was synthesized from compound (1g) (0.800 g, 1.17 mmol) and compound (2e) (azide) (1.64 g, 4.68 mmol), CuSO₄ (29 mg, 0.117 mmol), Na ascorbate (11 mg, 0.058 mmol) in 50% THF/H₂O. The crude product obtained was purified using NPC using MeOH/DCM to get brownish liquid (0.910 g, 0.877 mmol, 75%), R_{f} value=0.42 in 5% MeOH/DCM.

¹H NMR (400 MHZ, CDCl₃) δ : 7.48 (s, 1H), 6.45 (s, 2H), 5.37 (s, 2H), 4.65 (s, 2H), 4.07 (ddd, J=10.5, 5.4, 3.0 Hz, 4H), 3.91 (td, J=6.5, 5.0 Hz, 6H), 3.72-3.58 (m, 14H), 2.17-2.06 (m, 2H), 1.79-1.68 (m, 6H), 1.42 (dd, J=10.0, 5.0 Hz, 6H), 1.33-1.24 (m, 55H), 0.87 (t, J=6.8 Hz, 9H).

¹³C NMR (100 MHz, CDCl₃) δ : 153.69, 138.61, 129.48, 106.90, 73.58, 70.69, 70.66, 70.61, 70.57, 70.28, 69.88, 69.37, 65.24, 64.85, 61.78, 61.71, 61.59, 61.53, 54.61, 32.05, 31.87, 30.44, 29.87, 29.85, 29.82, 29.78, 29.76, 29.72, 29.54, 29.51, 29.49, 27.78, 26.40, 26.23, 26.20, 22.81, 18.23, 16.62, 16.57, 16.51, 14.23, 6.65.

MALDI-TOF MS (M+K): 1077.45 g/mol.

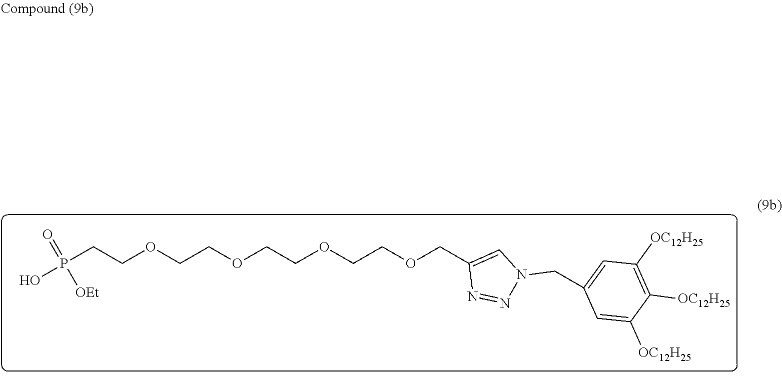

Mol. formula: C₅₆H₁₀₄N₃O₁₀P Mol. weight: 1010.43 g/mol
Physical appearance: pale yellow liquid
Yield: not determined

The compound (9b) was synthesized from compound (9a) (0.850 g, 0.818 mmol), OxCI (0.412 g, 3.27 mmol) in DCM. The product was carried to the next step without purification.

MALDI-TOF MS (M+Na): 1033.67 g/mol.

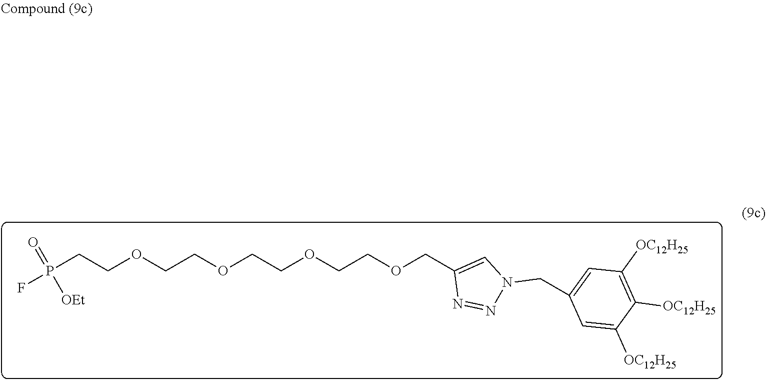

Mol. formula: C₅₆H₁₀₃FN₃O₉P Mol. weight: 1012.42 g/mol
Physical appearance: brownish liquid
Yield: not determined
   The compound (9c) was synthesized from compound (9b) (0.650 g, 0.643 mmol), DAST (0.413 g, 2.57 mmol) in DCM. The product was used for modification without further purification.

¹⁹F NMR (400 MHz, CDCl₃) δ: −59.89, −62.72.

MALDI-TOF MS (M+K): 1051.89 g/mol.

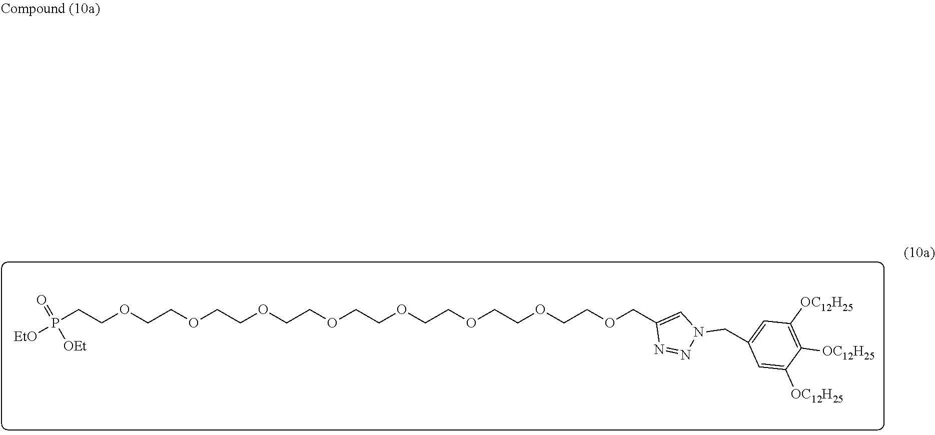

Mol. formula: C₆₆H₁₂₄N₃O₁₄P Mol. weight: 1214.70 g/mol
Physical appearance: brownish liquid
Yield: 62%
   The compound (10a) was synthesized from compound (1h) (1.09 g, 2.06 mmol) and compound (2e) (5.64 g, 8.24 mmol), CuSO₄ (51 mg, 0.206 mmol), Na ascorbate (20 mg, 0.103 mmol) in 50% THF/H₂O. The crude product obtained was purified by NPC using MeOH/DCM to get brownish liquid (1.55 g, 1.27 mmol, 62%), R_{f} value=0.40 in 5% MeOH/DCM.

¹H NMR (400 MHZ, CDCl₃) δ : 7.49 (s, 1H), 6.45 (s, 2H), 5.38 (s, 2H), 4.65 (s, 2H), 4.09 (ttd, J=13.9, 7.0, 3.7 Hz, 4H), 3.91 (td, J=6.5, 4.9 Hz, 6H), 3.74-3.55 (m, 30H), 2.12 (dt, J=
18.7, 7.6 Hz, 2H), 1.79 (d, J=7.2 Hz, 2H), 1.70 (s, 4H), 1.44 (dd, J=9.2, 4.7 Hz, 6H), 1.33-1.23
(m, 55H), 0.90-0.85 (m, 9H).

¹³C NMR (100 MHz, CDCl₃) δ : 153.71, 138.62, 129.52, 122.61, 106.91, 73.61, 70.72, 70.68,
70.62, 70.58, 70.32, 69.89, 69.39, 65.27, 64.86, 61.81, 61.74, 54.62, 32.07, 30.46, 29.89, 29.87,
29.84, 29.80, 29.78, 29.74, 29.56, 29.53, 29.51, 27.81, 26.42, 26.25, 26.22, 22.83, 16.59, 16.53,
14.26.

MALDI-TOF MS (M+K): 1253.76 g/mol.

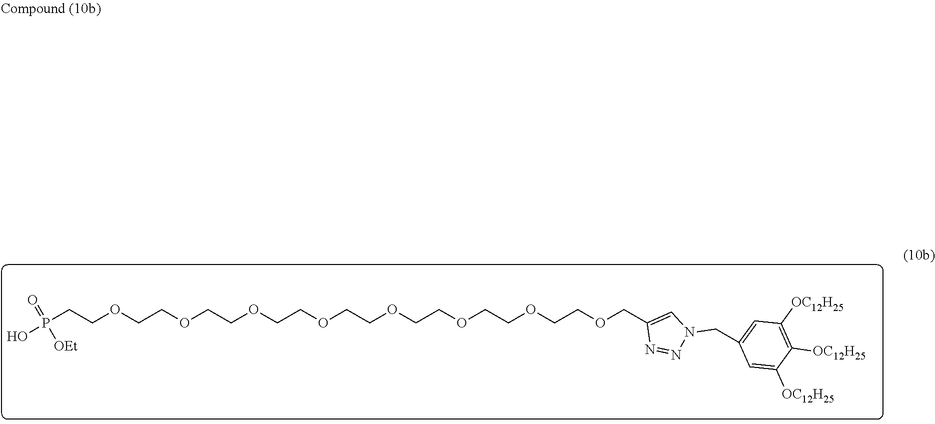

Mol. formula: C₆₄H₁₂₀N₃O₁₄P Mol. weight: 1186.64 g/mol
Physical appearance: pale yellow liquid
Yield: not determined
   The compound (10b) was synthesized from compound (10a) (0.500 g, 0.412 mmol), oxalyl chloride (0.207 g, 1.64 mmol) in DCM. The product was carried to the next step without purification.

MALDI-TOF MS (M+Na): 1209.50 g/mol.

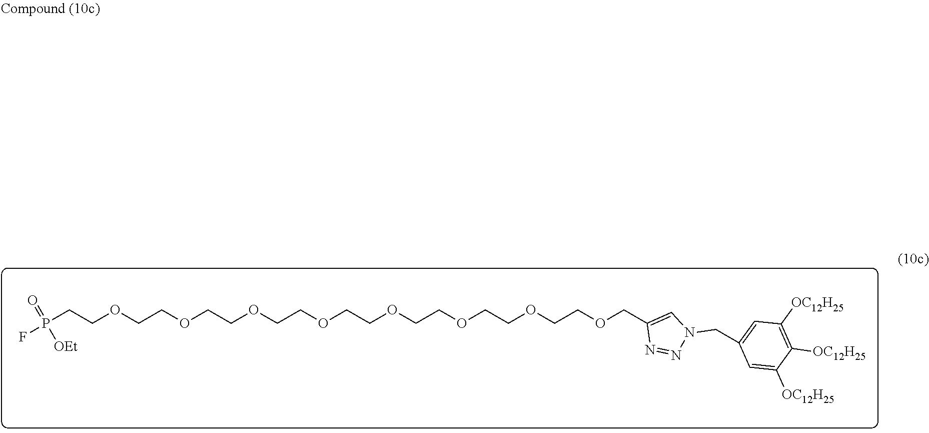

Mol. formula: C₆₄H₁₁₉FN₃O₁₃P Mol. weight: 1188.64 g/mol
Physical appearance: brownish liquid
Yield: not determined
   The compound (10c) was synthesized from compound (10b) (0.360 g, 0.303 mmol), DAST (0.195 g, 1.21 mmol) in DCM. The product was used for modification without further purification. ¹⁹F NMR (400 MHZ, CDCl₃) δ: −59.91, −62.74.

MALDI-TOF MS (M+K): 1227.76 g/mol.

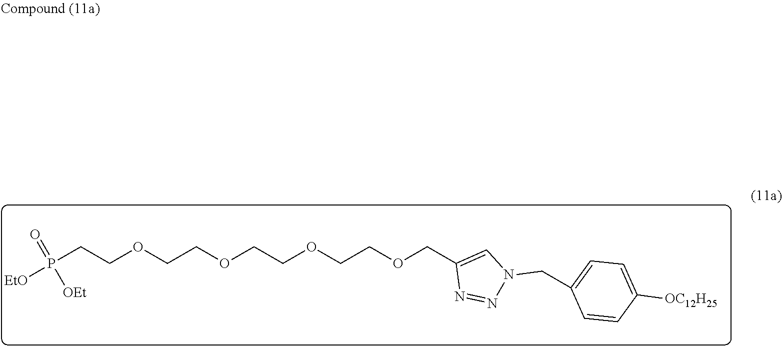

Mol. formula: C₃₄H₆₀N₃O₈P Mol. weight: 669.64 g/mol
Physical appearance: brownish liquid
Yield: 62%
   The compound (11a) was synthesized from compound (1g) (0.240 g, 0.681 mmol) and compound (3c) (0.864 g, 2.72 mmol), CuSO₄ (17 mg, 0.068 mmol), Na ascorbate (7 mg, 0.034 mmol) in 50% THF/H₂O. The crude product obtained was purified using NPC using MeOH/DCM
to get brownish liquid (0.282 g, 0.422 mmol, 62%), R_{f} value=0.40 in 5 MeOH/DCM.

¹H NMR (400 MHZ, CDCl₃) δ : 7.44 (s, 1H), 7.17 (d, J=8.3 Hz, 2H), 6.83 (d, J=8.5 Hz, 2H),
5.39 (s, 2H), 4.60 (s, 2H), 4.06 (dtq, J=11.0, 7.2, 4.0, 3.4 Hz, 4H), 3.90 (t, J=6.6 Hz, 2H), 3.74-3.52 (m, 15H), 2.08 (dt, J=18.7, 7.5 Hz, 2H), 1.74 (d, J=7.2 Hz, 2H), 1.40 (dq, J=
14.2, 6.5 Hz, 2H), 1.27 (p, J=6.6 Hz, 19H), 0.84 (t, J=6.7 Hz, 3H).

¹³C NMR (100 MHz, CDCl₃) δ : 159.52, 129.71, 126.32, 115.01, 70.57, 70.53, 70.48, 70.44,
70.18, 69.70, 68.14, 65.11, 64.68, 61.72, 61.66, 53.75, 31.92, 29.71, 29.67, 29.64, 29.60, 29.58,
29.39, 29.35, 29.21, 27.64, 26.25, 26.03, 22.70, 16.47, 16.41, 14.14.

MALDI-TOF MS (M+Na): 692.52 g/mol.

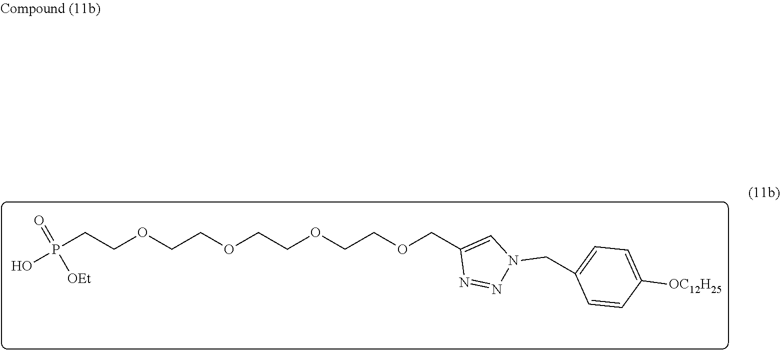

Mol. formula: C₃₂H₅₆N₃O₈P Mol. weight: 641.49 g/mol
Physical appearance: pale yellow liquid
Yield: not determined
   The compound (11b) was synthesized from compound (11a) (0.250 g, 0.373 mmol), oxalyl chloride (0.189 g, 1.49 mmol) in DCM. The product was carried to the next step without purification. MALDI-TOF MS (M+Na): 664.51 g/mol.

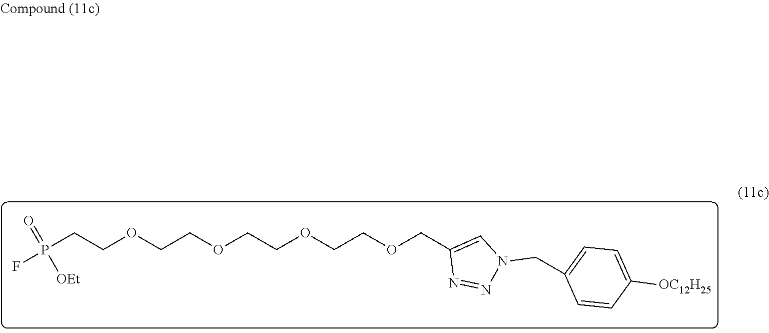

Mol. formula: C₃₂H₅₅FN₃O₇P Mol. weight: 643.78 g/mol
Physical appearance: brownish liquid
Yield: not determined
   The compound (11c) was synthesized from compound (11b) (0.170 g, 0.265 mmol), and DAST (0.170 g, 1.06 mmol) in DCM. The product was used for modification without further purification.

¹⁹F NMR (400 MHZ, CDCl₃) δ: −59.98, −62.81.

MALDI-TOF MS (M+Na): 666.97 g/mol.

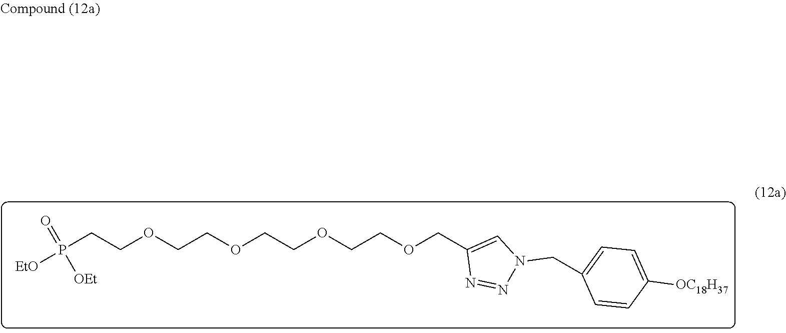

Mol. formula: C₄₀H₇₂N₃O₈P
Mol. weight: 754.00 g/mol
Physical appearance: brownish liquid
Yield: 65%
   The compound (12a) was synthesized from compound (1g) (0.240, 0.680 mmol) and compound (4c) (1.09 g, 2.72 mmol), CuSO₄ (17 mg, 0.068 mmol), Na ascorbate (7 mg, 0.034 mmol) in 50% THF/H₂O. The crude product obtained was purified using NPC using MeOH/DCM to get brownish liquid (0.333 g, 0.442 mmol, 65%), R_{f} value=0.40 in 5% MeOH/DCM. ¹H NMR (400 MHZ, CDCl₃) δ: 7.45 (s, 1H), 7.23-7.16 (m, 2H), 6.91-6.81 (m, 2H), 5.41 (s, 2H), 4.62 (s, 2H), 4.17-4.00 (m, 4H), 3.92 (t, J=6.5 Hz, 2H), 3.76-3.50 (m, 14H), 2.10 (dt, J=18.8, 7.5 Hz, 3H), 1.75 (dq, J=8.3, 6.5 Hz, 2H), 1.45-1.38 (m, 3H), 1.32-1.23 (m, 31H), 0.92-0.80 (m, 4H).

¹³C NMR (100 MHz, CDCl₃) δ : 159.61, 129.79, 126.34, 122.48, 115.08, 70.64, 70.60, 70.56, 70.51, 70.25, 69.78, 68.22, 65.19, 65.04, 64.76, 61.79, 61.72, 53.84, 34.90, 32.01, 31.58, 30.25, 29.78, 29.74, 29.69, 29.66, 29.48, 29.44, 29.34, 29.28, 28.67, 27.71, 26.33, 26.11, 25.99, 22.77, 16.53, 16.47, 14.21.

MALDI-TOF MS (M+Na): 777.7 g/mol.

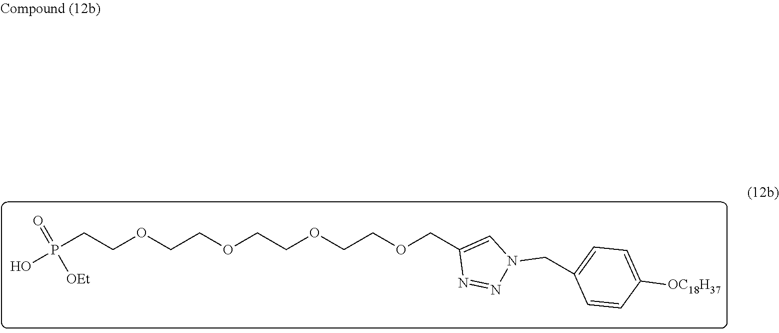

Mol. formula: C₃₈H₆₈N₃O₈P
Mol. weight: 725.95 g/mol
Physical appearance: pale yellow liquid
Yield: not determined
   The compound (12b) was synthesized from compound (11a) (0.560 g, 0.74 mmol), oxalyl chloride (0.377 g, 2.97 mmol) in DCM. The product was carried to the next step without purification.

MALDI-TOF MS (M+Na): 748.65 g/mol.

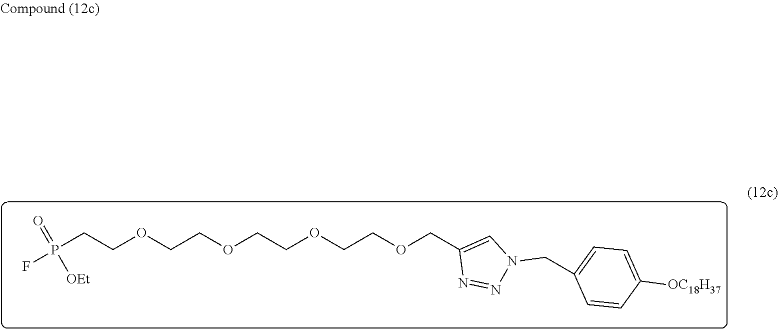

Mol. formula: C₃₈H₆₇FN₃O₇P
Mol. weight: 727.94 g/mol
Physical appearance: brownish liquid
Yield: not determined
   The compound (12c) was synthesized from compound (12b) (0.630 g, 0.868 mmol), DAST (0.559, 3.47 mmol) in DCM. The product was used for modification without further purification.

¹⁹F NMR (400 MHZ, CDCl₃) δ: −59.92, −62.75.

MALDI-TOF MS (M+Na): 750.23 g/mol.

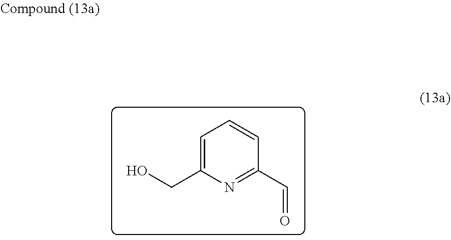

Mol. formula: C₇H₇NO₂
Mol. Weight: 137.14 g/mol
Physical appearance: white solid
Yield: 60%
   To a solution of 2,6-pyridinedimethanol (7 g, 50.3 mmol) in 1,4-dioxane (10 ml), SeO₂ (2.7 g, 25.15 mmol) was refluxed at 80° C. for 24 hrs. The resulting crude material was purified by NPC using MeOH/DCM as an eluent to get a liquid that became an off-white solid later (2.06 g, 15.09 mmol, 60%), R_{f} Value=0.45 in 5% MeOH/DCM.

¹H NMR (400 MHZ, CDCl₃) δ : 10.05 (d, J=0.6 Hz, 1H), 7.92-7.79 (m, 2H), 7.58-7.46 (m, 1H), 4.86 (s, 2H), 3.67 (s, 1H).

¹³C NMR (100 MHZ, CDCl₃) δ : 192.82, 160.09, 160.06, 151.37, 137.84, 137.56, 124.68, 120.79, 120.35, 69.23, 66.82, 64.35, 63.91, 53.23.

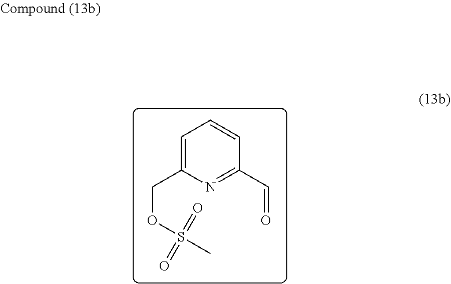

Mol. formula: C₈H₉NO₄S
Mol. Weight: 137.13 g/mol

Physical appearance: white solid
Yield: 60%
   methane sulfonyl chloride (2.94 g, 20.4 mmol), and Et₃N (4.41 g, 43.7 mmol) was added to the solution of alcohol compound (13a) (2 g, 14.58 mmol) in DCM (10 ml) stirred for 16 hrs at RT to get crude material was purified by NPC using EtOAc/Hexane as an eluent to get white solid as a pure product (1.19 g, 8.7 mmol, 60%), R_{f} Value=0.45 in 50% EtOAc/Hexane

¹H NMR (400 MHZ, CDCl₃) δ : 9.99 (d, J=0.8 Hz, 1H), 7.95-7.83 (m, 2H), 7.68 (dd, J=7.2, 1.6 Hz, 1H), 4.71 (s, 2H).

¹³C NMR (100 MHz, CDCl₃) δ : 192.98, 192.39, 157.45, 152.29, 138.27, 137.99, 127.14, 46.07.

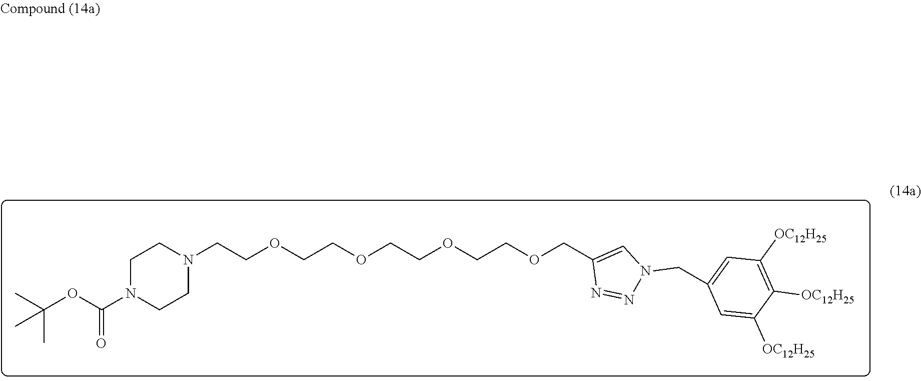

Mol. formula: C₆₃H₁₁₅N₅O₉
Mol. weight: 1086.64 g/mol
Physical appearance: brownish liquid
Yield: 65%
   The compound (14a) was synthesized from compound (1i) (1.5 g, 3.75 mmol) and compound (2e) (10.2 g, 15 mmol), CuSO₄ (93 mg, 0.375 mmol), Na ascorbate (37 mg, 0.187 mmol) in 50% THF/H₂O. The crude product obtained was purified using NPC using MeOH/DCM to get brownish liquid (2.64 g, 2.43 mmol, 65%), R_{f} value=0.40 in 5% MeOH/DCM.

¹H NMR (400 MHZ, CDCl₃) δ: 7.47 (s, 1H), 6.44 (s, 2H), 5.36 (s, 2H), 4.64 (s, 2H), 3.90 (q, J=6.2 Hz, 6H), 3.69-3.55 (m, 14H), 3.42 (t, J=5.1 Hz, 4H), 2.58 (t, J=5.7 Hz, 2H), 2.44 (t, J=5.1 Hz, 4H), 1.79-1.66 (m, 6H), 1.49-1.36 (m, 15H), 1.25 (d, J=8.0 Hz, 49H), 0.86 (t, J=6.7 Hz, 9H).

¹³C NMR (100 MHZ, CDCl₃): 154.79, 153.65, 145.53, 138.56, 129.46, 122.53, 114.15, 106.85, 79.69, 73.54, 70.63, 70.58, 70.43, 69.84, 69.32, 68.74, 64.80, 57.87, 54.55, 53.41, 33.90, 32.02, 32.00, 30.40, 29.83, 29.81, 29.79, 29.77, 29.74, 29.72, 29.68, 29.50, 29.47, 29.45, 29.03, 28.50, 26.19, 26.17, 22.77, 14.20, 0.08.

MALDI-TOF MS (M+Na): 1109.69 g/mol.

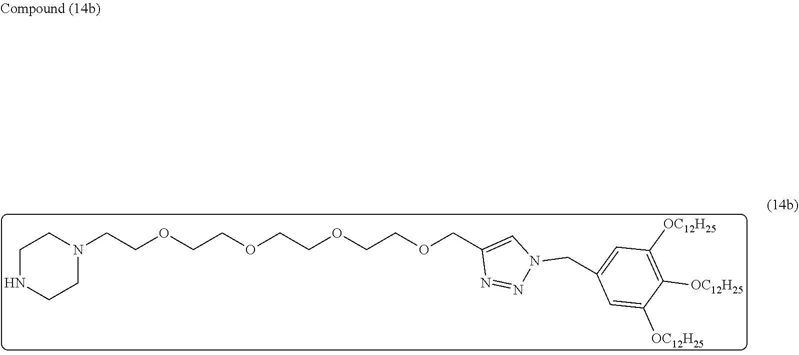

Mol. formula: C₅₈H₁₀₇N₅O₇
Mol. weight: 1037.78 g/mol
Physical appearance: brownish liquid
Yield: Not determined
   compound (14a) dissolved in DCM and 2 ml Conc HCl was added to the reaction mixture and stirred for two hrs. After two hrs reaction mixture was quenched with the addition of aq. NaOH solution and extracted by DCM. The organic layer is concentrated and crude used as it is in next step.

MALDI-TOF MS (M+Na): 1060.87 g/mol.

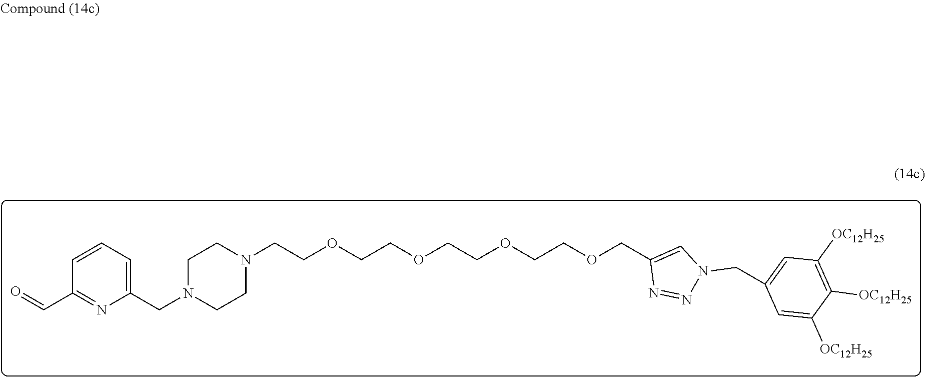

Mol. formula: C₆₅H₁₁₂N₆O₈
Mol. weight: 1105.65 g/mol
Physical appearance: brownish liquid
Yield: 35%
   Crude Compound (14b) (1.6 g, 1.54 mmol), K₂CO₃ (0.837 g, 6.16 mmol), and mesylate compound (13b) (1.32 g, 6.16 mmol) were weighed in RBF. Then the mixture was dissolved in ACN (10 ml) and refluxed at 65° C. After 16 hrs, the reaction mixture was concentrated and purified by NPC using MeOH/DCM as an eluent system, (0.592 g, 0.539 mmol, 35%), R_{f} value=0.33 in 10% MeOH/DCM.

¹H NMR (400 MHz, CDCl₃) δ : 10.03 (s, 1H), 7.80 (d, J=4.5 Hz, 2H), 7.65 (q, J=4.2 Hz, 1H), 7.47 (s, 1H), 6.43 (s, 2H), 5.35 (s, 2H), 4.62 (s, 2H), 3.88 (q, J=6.3 Hz, 6H), 3.73 (s, 2H), 3.68-3.52 (m, 14H), 2.58 (dd, J=11.0, 5.0 Hz, 9H), 1.80-1.63 (m, 6H), 1.41 (ddd, J=16.1, 10.4, 6.5 Hz, 6H), 1.29-1.21 (m, 48H), 0.84 (t, J=6.7 Hz, 10H).

¹³C NMR (100 MHz, CDCl₃) δ : 193.66, 159.70, 153.60, 152.38, 145.46, 139.26, 138.50, 137.38, 129.43, 127.49, 122.51, 120.21, 114.11, 106.79, 73.48, 70.57, 70.52, 70.36, 69.78, 69.26, 68.74, 64.74, 64.00, 57.73, 54.50, 53.51, 53.08, 33.85, 31.97, 31.96, 31.71, 31.54, 31.46, 30.36, 30.16, 29.78, 29.76, 29.74, 29.69, 29.67, 29.63, 29.54, 29.45, 29.42, 29.40, 29.18, 28.98, 26.15, 26.12, 22.72, 14.16.

MALDI-TOF MS (M+Na): 1128.79 g/mol.

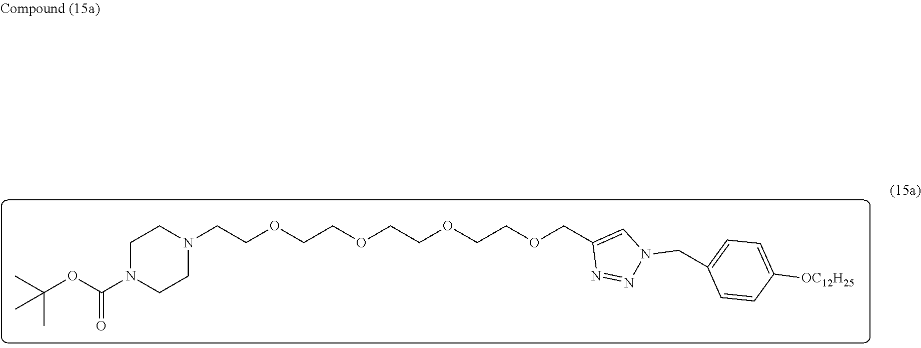

Mol. formula: C₃₉H₆₇N₅O₇
Mol. weight: 717.99 g/mol
Physical appearance: brownish liquid
Yield: 65%
   The compound (15a) was synthesized from compound (1i) (0.540 g, 1.34 mmol) and compound (3c) (1.7 g, 5.39 mmol), CuSO₄ (33 mg, 0.134 mmol), Na ascorbate (13 mg, 0.067 mmol) in 50% THF/H₂O. The crude product obtained was purified using NPC using MeOH/DCM to get brownish liquid (0.625 g, 0.871 mmol, 65%), R_{f} value=0.32 in 10% MeOH/DCM.

¹H NMR: (400 MHZ, CDCl₃) δ 7.41 (s, 1H), 7.19-7.09 (m, 2H), 6.81 (dd, J=9.0, 2.3 Hz, 2H), 5.37 (s, 2H), 4.58 (d, J=2.0 Hz, 2H), 3.87 (t, J=6.6 Hz, 2H), 3.62-3.48 (m, 14H), 3.37 (t, J=5.1 Hz, 4H), 2.53 (t, J=5.7 Hz, 2H), 2.38 (t, J=5.1 Hz, 4H), 1.70 (dt, J=12.9, 6.6 Hz, 3H), 1.39 (d, J=2.3 Hz, 12H), 1.26-1.17 (m, 16H), 0.86-0.76 (m, 3H).

¹³C NMR: (100 MHz, CDCl₃) δ 159.44, 154.65, 145.35, 129.62, 126.28, 122.26, 114.92, 79.48, 70.51, 70.49, 70.44, 70.30, 69.64, 68.70, 68.67, 68.04, 64.63, 57.75, 53.63, 53.28, 33.76, 31.85, 31.65, 31.46, 30.09, 29.63, 29.59, 29.57, 29.53, 29.51, 29.32, 29.28, 29.14, 28.88, 28.37, 25.96, 22.62, 14.08.

MALDI-TOF MS (M+Na): 740.64 g/mol.

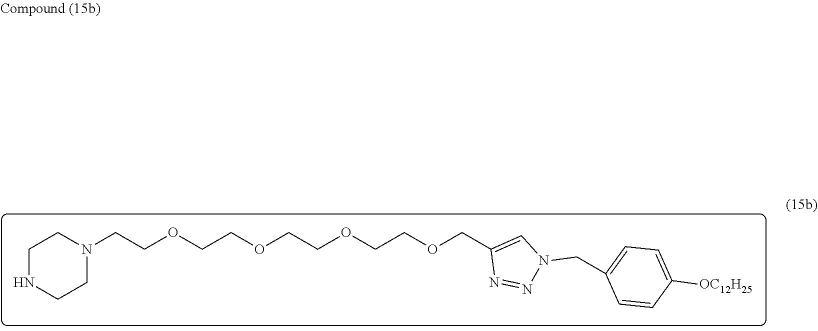

Mol. formula: C₃₄H₅₉N₅O₅
Mol. weight: 617.88 g/mol
Physical appearance: brownish
liquid Yield: Not determined
   compound (14a) dissolved in DCM and 2 ml Conc HCl was added to the reaction mixture and stirred for two hrs. After two hrs reaction mixture was quenched by the addition of aq. NaOH solution and extracted by DCM. The organic layer is concentrated and crude used as it is in the next step.

MALDI-TOF MS (M+Na): 640.65 g/mol.

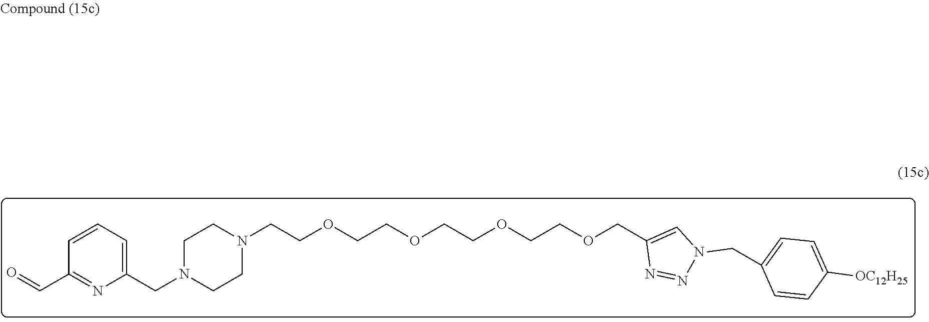

Mol. formula: C₄₁H₆₄N₆O₆
Mol. weight: 717.99 g/mol
Physical appearance: brownish
liquid Yield: 40%
   Crude compound (15b) (0.263 g, 0.427 mmol), K₂CO₃ (0.231 g, 1.70 mmol) and mesylate compound (13b) (0.365 g, 1.70 mmol) were weighed in RBF. Then the mixture was dissolved in ACN (20 ml) and refluxed at 65° C. After 16 hrs, the reaction mixture was concentrated and purified by NPC using MeOH/DCM as an eluting system, (0.122 g, 0.170 mmol, 40%), R_{f} value=0.32 in 10% MeOH/DCM.

¹H NMR (400 MHZ, CDCl₃) δ : 10.00 (s, 1H), 7.85-7.76 (m, 1H), 7.46 (d, J=2.0 Hz, 1H), 7.21-7.10 (m, 2H), 6.86-6.75 (m, 2H), 5.38 (s, 2H), 4.57 (s, 2H), 3.88 (t, J=6.5 Hz, 2H), 3.72 (s, 2H), 3.65-3.51 (m, 14H), 2.98 (s, 7H), 2.62 (d, J=5.6 Hz, 4H), 1.71 (p, J=6.7 Hz, 2H), 1.42-1.35 (m, 2H), 1.21 (d, J=6.6 Hz, 16H), 0.82 (t, J=6.7 Hz, 3H).

¹³C NMR (100 MHZ, CDCl₃) δ : 193.59, 159.49, 159.24, 152.29, 145.21, 137.71, 137.55, 129.69, 127.70, 126.23, 123.50, 122.55, 122.52, 120.40, 119.98, 114.99, 114.05, 96.38, 70.44, 70.41, 70.38, 70.22, 70.20, 69.65, 68.17, 68.13, 64.49, 63.72, 63.69, 57.53, 57.46, 54.67, 53.74, 53.26, 53.05, 52.60, 52.43, 50.29, 49.14, 33.79, 31.88, 31.64, 29.66, 29.62, 29.60, 29.56, 29.54, 29.35, 29.31, 29.16, 28.92, 25.99, 22.65, 14.09.

MALDI-TOF MS (M+Na): 740.44 g/mol.

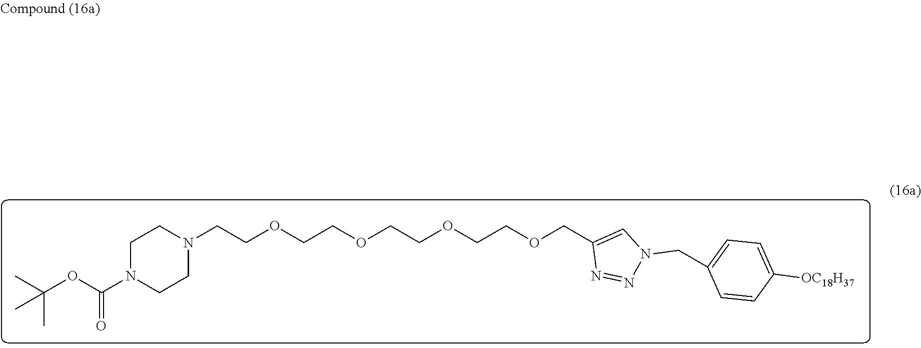

Mol. formula: C₄₅H₇₉N₅O₇
Mol. weight: 802.16 g/mol
Physical appearance:
White solid Yield: 65%
   The compound (16a) was synthesized from compound (1i) (0.600, 1.49 mmol) and compound (4c) (2.4 g, 5.99 mmol), CuSO₄ (37 mg, 0.149 mmol), Na ascorbate (1 mg, 0.074 mmol) in 50% THF/H₂O. The crude product obtained was purified using NPC using MeOH/DCM to get brownish liquid (0.776 g, 0.968 mmol, 65%), R_{f} value=0.40 in 10% MeOH/DCM.

¹H NMR (400 MHZ, CDCl₃) δ : 7.43 (s, 1H), 7.23-7.14 (m, 2H), 6.90-6.80 (m, 2H), 5.41 (s, 2H), 4.62 (s, 2H), 3.91 (t, J=6.6 Hz, 2H), 3.69-3.54 (m, 14H), 3.41 (t, J=5.0 Hz, 4H), 2.56 (t, J=5.8 Hz, 2H), 2.41 (t, J=5.1 Hz, 4H), 1.79-1.71 (m, 2H), 1.43 (s, 12H), 1.23 (s, 31H), 0.85 (t, J=6.7 Hz, 3H).

¹³C NMR (100 MHz, CDCl₃) δ : 159.61, 154.83, 129.78, 126.36, 122.40, 115.08, 79.67, 70.67, 70.65, 70.63, 70.59, 70.45, 69.79, 68.84, 68.22, 64.79, 57.89, 53.83, 53.42, 32.01, 31.59, 29.78, 29.75, 29.69, 29.66, 29.48, 29.45, 29.29, 28.52, 26.11, 22.78, 14.22, 0.09.

MALDI-TOF MS (M+Na): 825.8 g/mol.

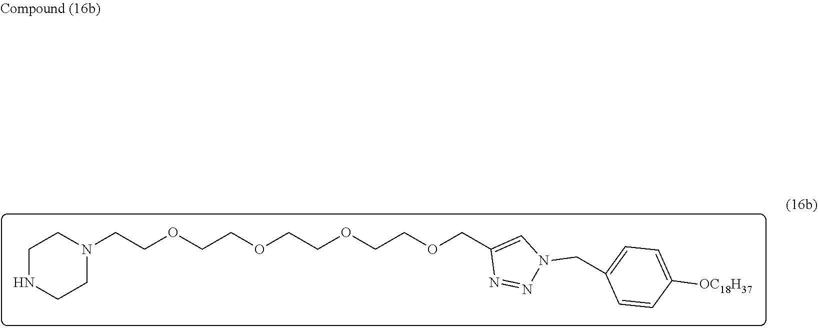

Mol. formula: C₄₀H₇₁N₅O₅
Mol. weight: 702.04 g/mol
Physical appearance: White solid
Yield: 62%
   Compound (16a) dissolved in DCM and 2 ml Conc HCl was added to the reaction mixture and stirred for two hrs. After two hrs reaction mixture was quenched with the addition of aq. NaOH solution and extracted by DCM. The organic layer is concentrated and crude used as it is in the next step.

MALDI-TOF MS (M+Na): 725.98 g/mol.

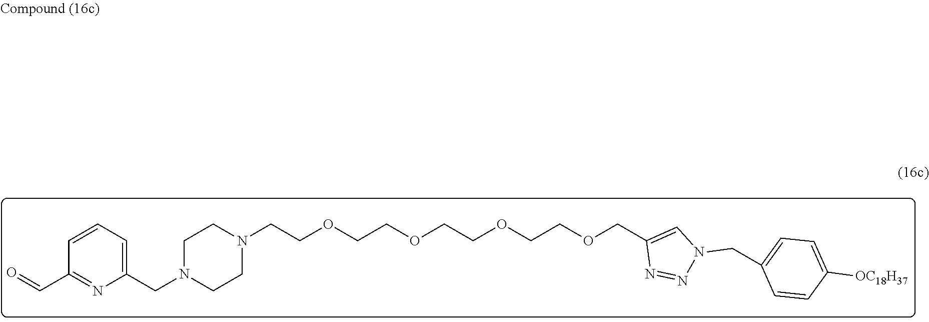

Mol. formula: C₄₇H₇₆N₆O₆
Mol. weight: 821.16 g/mol
Physical appearance: White
solid Yield: 35%
   Crude Compound (16b) (1.3 g, 1.85 mmol), K₂CO₃ (1.00 g, 7.4 mmol), and mesylate compound (13b) (1.59 g, 7.4 mmol) were weighed in RBF. Then the mixture was dissolved in ACN (10 ml) and refluxed at 65° C. After 16 hrs, the reaction mixture was concentrated and purified by NPC using MeOH/DCM as an eluting system, (0.531 g, 0.647 mmol, 35%), R_{f} value=0.39 in 10% MeOH/DCM.

¹H NMR (400 MHZ, CDCl₃) δ : 10.02 (s, 1H), 7.79 (d, J=4.5 Hz, 2H), 7.64 (q, J=4.2 Hz, 1H), 7.43 (s, 1H), 7.21-7.12 (m, 2H), 6.88-6.77 (m, 2H), 5.39 (s, 2H), 4.59 (s, 2H), 3.89 (t, J=6.6 Hz, 2H), 3.73 (s, 2H), 3.61 (dd, J=5.6, 2.7 Hz, 2H), 3.57 (td, J=5.4, 2.9 Hz, 11H), 2.59 (t, J=5.8 Hz, 8H), 1.83-1.64 (m, 2H), 1.44-1.33 (m, 3H), 1.21 (s, 29H), 0.83 (t, J=6.7 Hz, 3H).

¹³C NMR (100 MHZ, CDCl₃) δ : 193.64, 159.56, 159.51, 152.35, 145.36, 137.42, 129.70, 127.54, 126.29, 123.33, 122.37, 121.53, 120.25, 119.93, 115.00, 70.52, 70.50, 70.47, 70.31, 69.69, 68.54, 68.13, 64.65, 63.91, 57.67, 54.68, 53.73, 53.43, 52.92, 52.76, 52.63, 51.68, 50.46, 49.50, 33.82, 31.93, 31.69, 31.51, 30.14, 29.70, 29.66, 29.61, 29.58, 29.40, 29.36, 29.20, 29.16, 28.95, 26.03, 22.69, 14.14.

MALDI-TOF MS (M+Na): 844.79 g/mol.

A skilled artisan will appreciate that the quantity and type of each ingredient can be used in different combinations or singly. All such variations and combinations would be falling within the scope of present disclosure.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Invention

The present disclosure provide a process for synthesizing self-assembling artificial proteins (SAPs).

Simplified Process: The invention eliminates the need for cumbersome and labor-intensive purification steps. Traditional SAP synthesis often involves multi-step reactions requiring extensive separation and purification of intermediates, leading to inefficiencies. By employing a catalytic host-guest system, the invention streamlines the process, saving time and resources while ensuring a high-quality product.

High Yield: Existing methods often suffer from poor yields due to losses during complex reaction setups and purification processes. The present invention enhances yield by minimizing side reactions, ensuring precise site-specific bioconjugation, and reducing material wastage. This improvement makes the process more viable for industrial-scale production, where maximizing output is critical.

Environmental Sustainability: The method minimizes the use of toxic reagents and solvents commonly employed in protein modification and assembly. By avoiding the generation of hazardous byproducts and reducing the need for environmentally damaging purification techniques, the invention supports sustainable and green chemistry practices. This is particularly advantageous for large-scale industrial applications where regulatory compliance and environmental impact are significant concerns.

Cost-Effectiveness: The reduced number of steps, minimal use of reagents, and elimination of costly purification procedures result in a cost-effective process. This makes the technology accessible for various industries, including pharmaceutical manufacturing, where economic feasibility plays a crucial role in determining the adoption of innovative techniques.

Versatility: The process is compatible with a range of proteins, including bovine serum albumin (BSA) and human serum albumin (HSA), serine proteases such as chymotrypsin, subtilisin, and/or proteinaseK and various catalytic host-guest systems, such as β-cyclodextrins. This adaptability enables customization based on specific applications, such as drug delivery, imaging, and vaccine development, broadening the scope of its applicability.

Improved Stability: Proteins synthesized using this method exhibit enhanced structural stability and functional integrity. Conventional approaches often result in unstable assemblies prone to disintegration under physiological conditions. By employing a precise and efficient bioconjugation process, this invention produces SAPs that maintain their functionality and stability, even under challenging conditions.

Precision: The invention ensures site-specific bioconjugation of proteins, which is critical for reliable and uniform self-assembly. This precision reduces the risk of nonspecific binding and aggregation, resulting in a more consistent product that can meet stringent regulatory standards, especially in therapeutic and diagnostic applications.

Broad Applications: The SAPs synthesized through this method can be utilized in various cutting-edge applications, including:

Therapeutics: Drug delivery systems, controlled release formulations, and targeted therapy for cancer and other diseases.

Diagnostics: Enhanced imaging agents for MRI, PET, and fluorescence imaging, as well as biosensors for disease detection.

Research: Platforms for studying protein interactions, molecular assembly, and bioengineering. This versatility makes the invention a valuable tool across biomedical, industrial, and academic research domains.

Scalability: The invention is designed with scalability in mind, making it suitable for both laboratory-scale experiments and industrial-scale manufacturing. The straightforward reaction setup and simplified purification requirements allow seamless scaling, ensuring consistent quality and performance irrespective of production volume.

Reproducibility: The method offers a high degree of reproducibility by standardizing key parameters such as reaction time, temperature, pH, and reagent concentrations. This consistency is crucial for applications requiring stringent quality control, such as pharmaceutical manufacturing and diagnostic tool production.

Enhanced Safety: By avoiding hazardous solvents and harsh reaction conditions, the invention improves safety for both operators and the environment. The reduced risk of exposure to toxic chemicals ensures compliance with workplace safety standards and minimizes liability concerns for manufacturers.

Competitive Edge: Compared to prior art, the invention offers a competitive advantage by combining efficiency, scalability, and sustainability.

Organizations adopting this technology can reduce production costs, shorten development timelines, and improve the marketability of their products.

Customizability: The method allows for easy modifications, enabling the synthesis of SAPs tailored to specific end-use requirements, such as size, surface properties, and functional groups. This feature makes the invention adaptable to a wide range of biomedical and industrial applications.

The outlined advantages demonstrate the significant improvements brought by this invention over conventional SAP synthesis methods. The combination of simplicity, precision, scalability, and sustainability positions this innovation as a game-changing technology in the fields of biotechnology, protein engineering, and nanomedicine. By addressing existing limitations, this invention paves the way for the broader adoption of SAPs in diverse therapeutic, diagnostic, and industrial applications.

Although the present invention has been described with reference to preferred embodiments, it is submitted that various modifications can be made to the exemplary embodiments without departing from the spirit and scope of the invention.

1. A method for synthesizing self-assembling artificial proteins (SAPs) comprising the steps of:
a. Dissolving the synthetic/chemical probe in an organic solvent to obtain solution A;
b. Adding 100 mmol γ-cyclodextrin and sodium phosphate buffer (pH 7.4) to solution A.
c. The above mixture was sonicated for 1-4 hrs at 25-60° C. to obtain solution B;
d. Protein solution was prepared in sodium phosphate buffer (pH 7.4) in a separate vessel and added to solution B;
e. The above reaction mixture was stirred for 30 mins-48 hours at 20-30° C.; and
f. Monitoring the reaction mixture using mass spectrometry and obtaining self-assembling artificial proteins (SAPs).

2. The method as claimed in claim 1, wherein the sonication is conducted for 2 hours at 40° C.

3. The method as claimed in claim 1, wherein the mixture of step (iv) is stirred at a speed of 20 rpm for 16 hours at 25° C.

4. The method as claimed in claim 1, wherein the protein is selected from bovine serum albumin (BSA), human serum albumin (HSA), chymotrypsin, subtilisin, and/or proteinase K.

5. The method as claimed in claim 1, wherein the cyclodextrin is α, β, or γcyclodextrin.

6. The method as claimed in claim 1, wherein the cyclodextrin is γcyclodextrin.

7. The method as claimed in claim 1, wherein the synthetic/chemical probe is selected from the following:

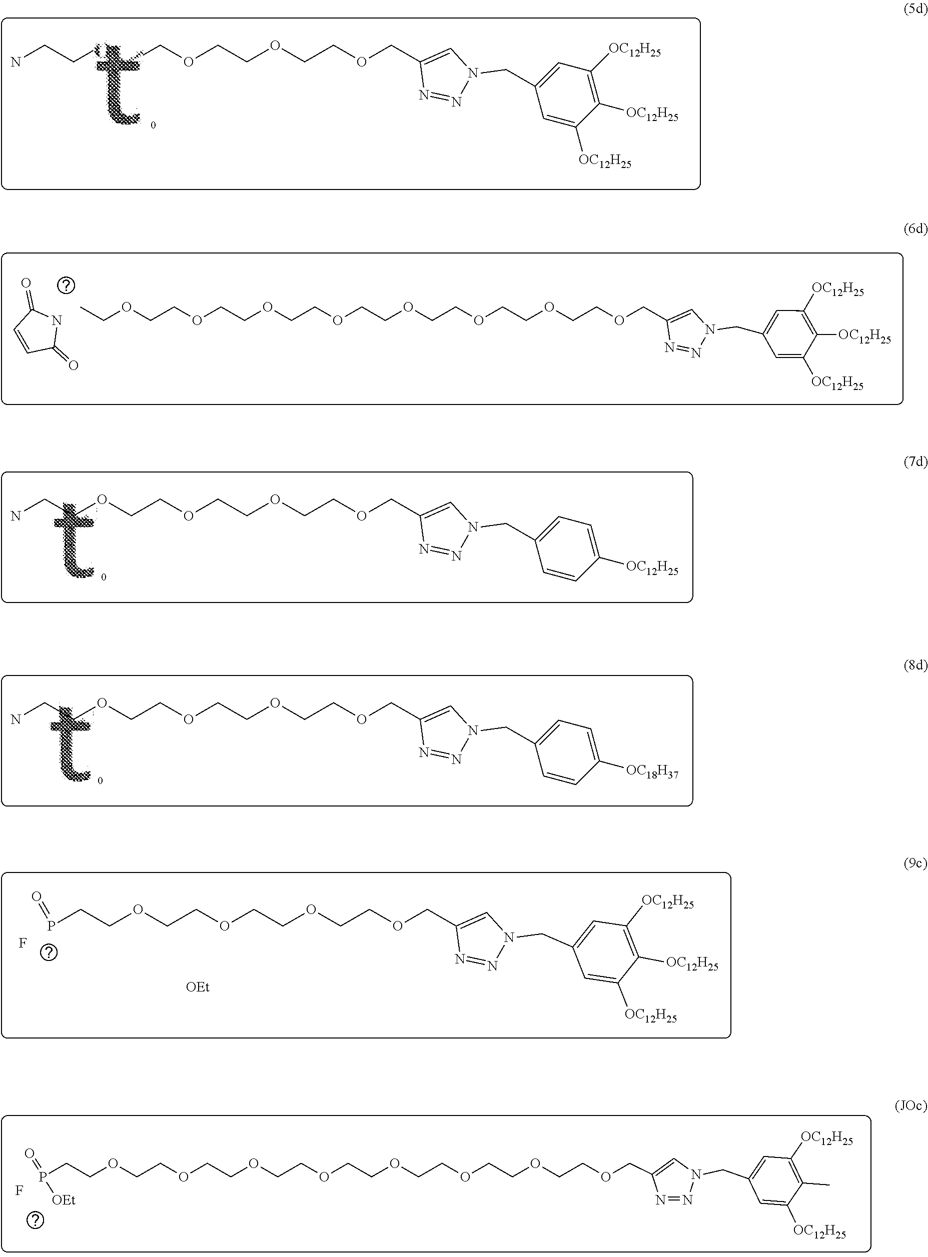

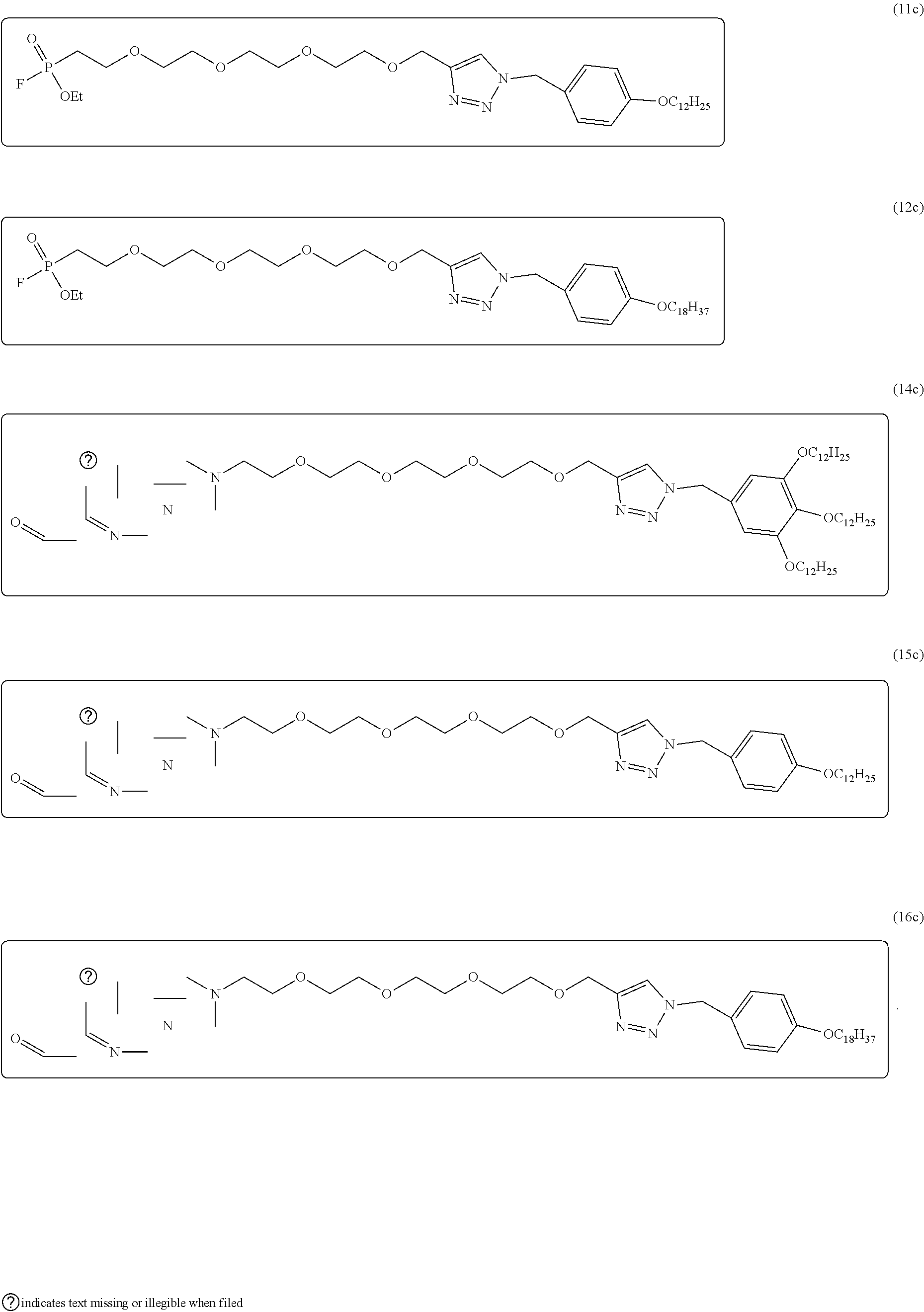

8. The method as claimed in claim 1, wherein the SAP is selected from the following:

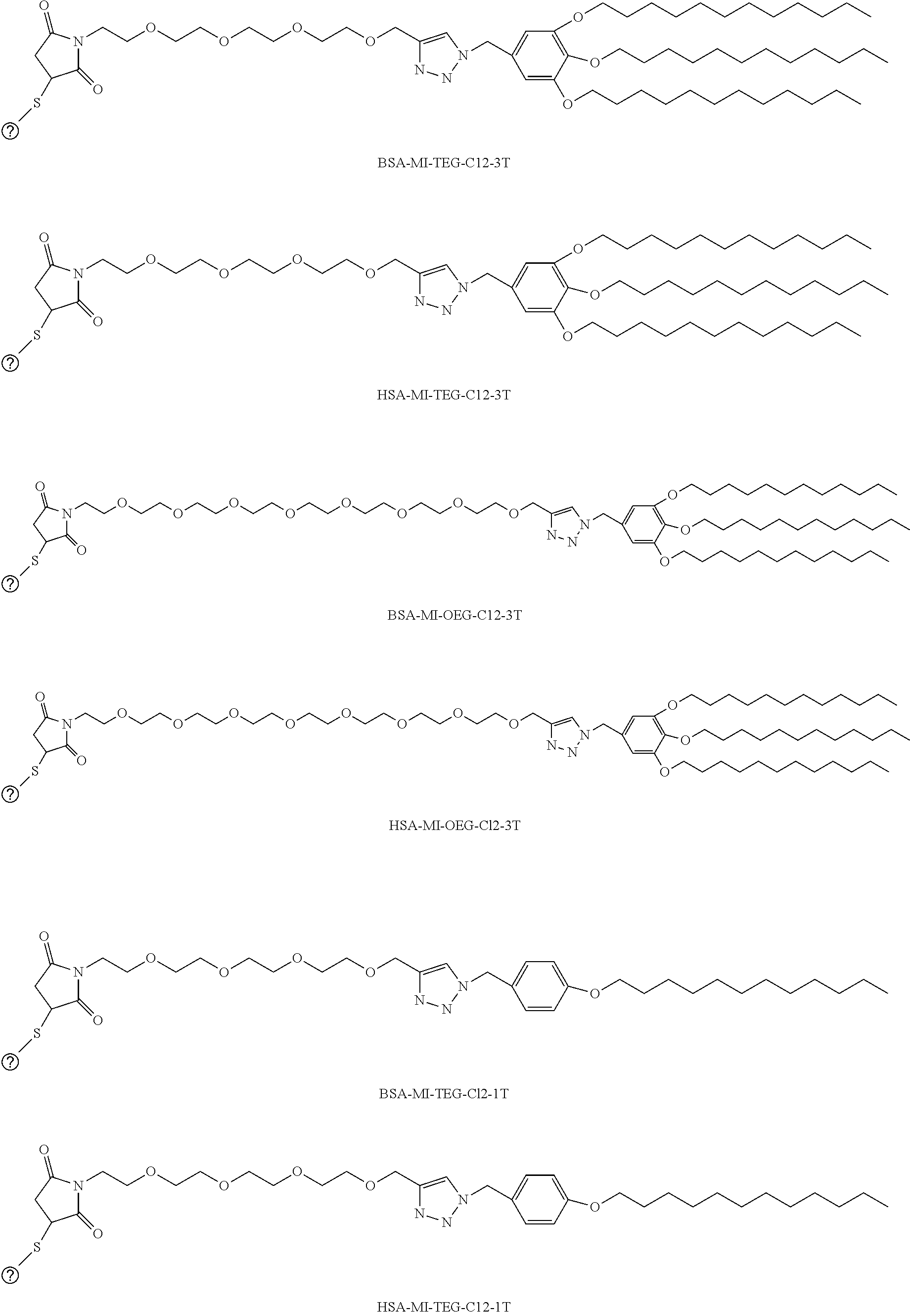

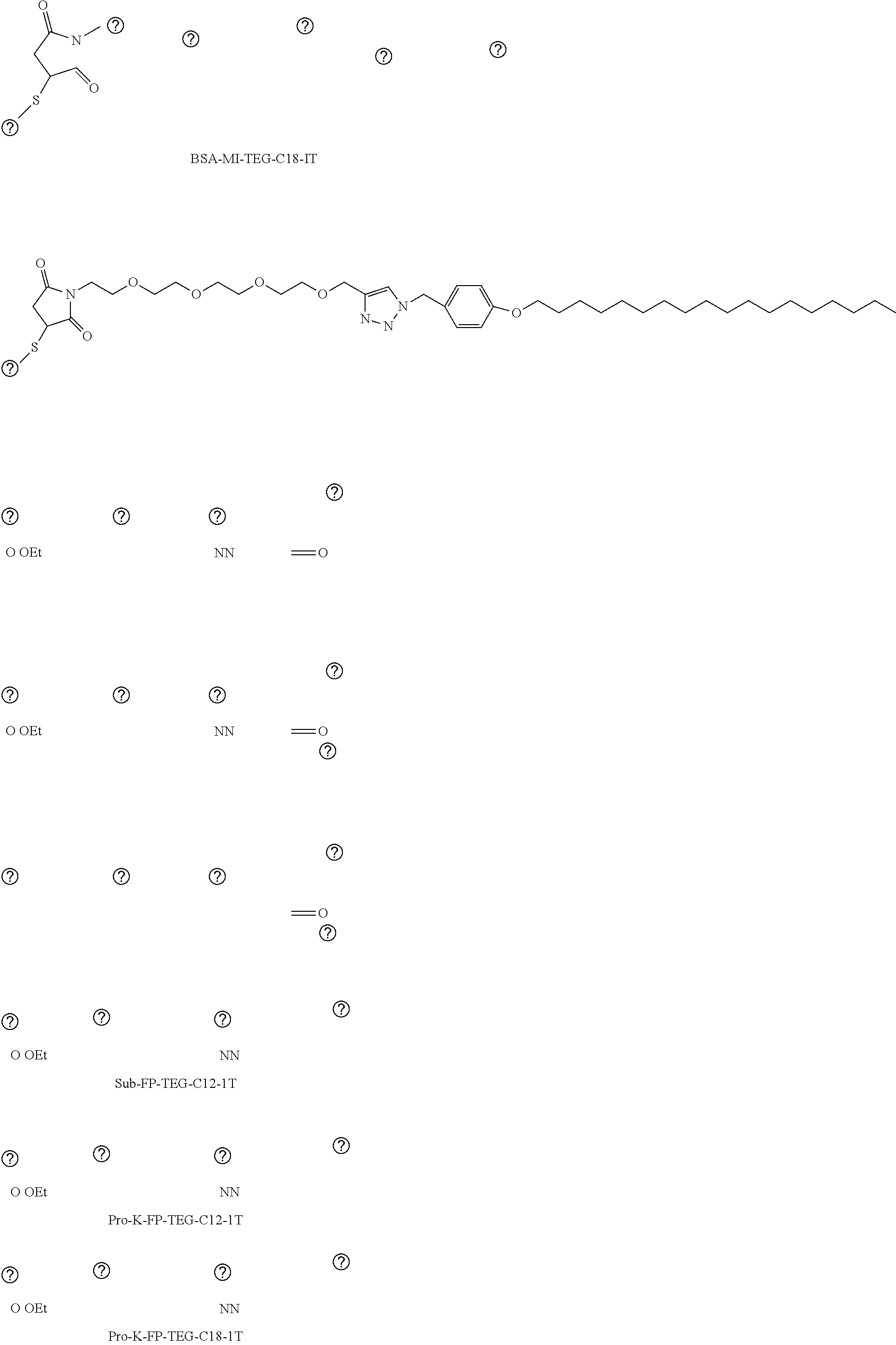

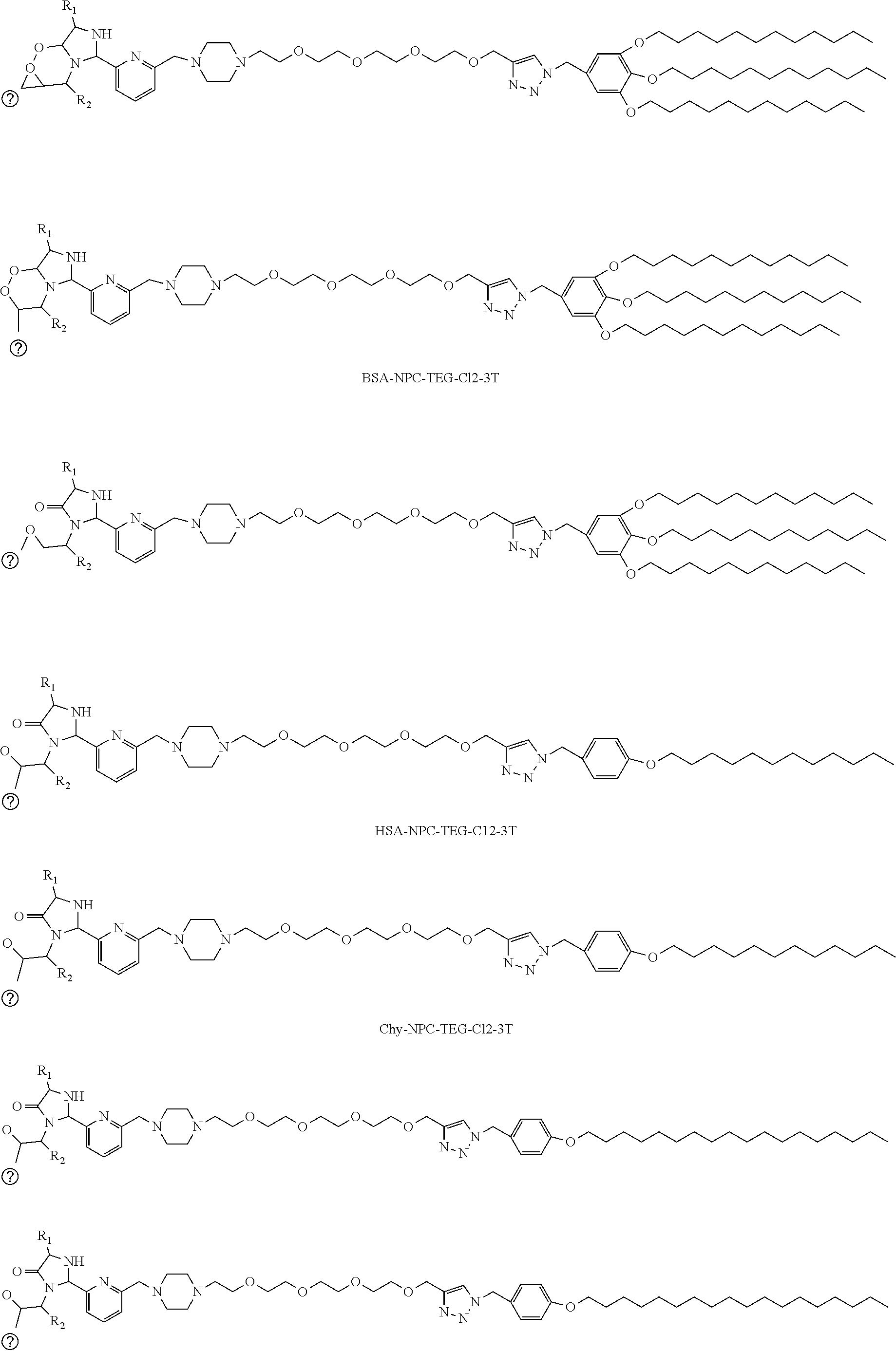

9. The method as claimed in claim 1, wherein the solvent is Tetrahydrofuran (THF)

10. A self-assembling artificial protein (SAP) synthesized by the method as claimed in claim 1.

11. A composition comprising self-assembling artificial proteins (SAPs) synthesized by the method as claimed in claim 1 and a pharmaceutically acceptable carrier.

12. The composition as claimed in claim 10, wherein the composition is configured for targeted drug delivery or imaging applications.
